(19) United States
(12) Patent Application Publication
(10) Pub. No.: US 2023/0227546 A1
(43) Pub. Date: Jul. 20, 2023

(54) TYPE 2 CYTOKINES AS PREDICTORS OF DISEASE SEVERITY AND/OR AS THERAPEUTIC TARGETS FOR COVID-19

(71) Applicant: University of Virginia Patent Foundation, Charlottesville, VA (US)

(72) Inventors: William A. Petri, JR., Charlottesville, VA (US); Alexandra N. Donlan, Charlottesville, VA (US); Mary Katherine Young, Charlottesville, VA (US); Mayuresh M. Abhyankar, Charlottesville, VA (US); Barbara J. Mann, Charlottesville, VA (US)

(21) Appl. No.: 18/011,412

(22) PCT Filed: Jun. 17, 2021
(86) PCT No.: PCT/US2021/037912

§ 371 Date: Dec. 19, 2022

Related U.S. Application Data
(60) Provisional application No. 63/129,145, filed on Dec. 22, 2020.
(60) Provisional application No. 63/073,234, filed on Sep. 1, 2020.
(60) Provisional application No. 63/040,254, filed on Jun. 17, 2020.

(51) Int. Cl. C07K 16/24; G01N 33/68; A61K 31/519

(52) U.S. Cl. CPC C07K 16/247; G01N 33/6869; A61K 31/519; C07K 2317/76; G01N 2333/165; G01N 2333/50; G01N 2333/5418; G01N 2333/5437; G01N 2800/56; G01N 2333/5412; G01N 2333/5421; G01N 2333/523

(57) ABSTRACT

Methods for treating coronavirus disease 2019 (COVID-19), the disease caused by severe acute respiratory syndrome coronavirus 2 (SARS-CoV-2) infections, is described. The methods can be used to reduce the severity of outcomes related to COVID-19, such as hospitalization and ventilation. For example, treatment of a subject with a therapeutic agent that neutralizes interleukin 13 (IL-13) can result in reduced risk for mechanical ventilation in the subject. Also described are methods of predicting risk of mechanical ventilation in subjects with COVID-19.

### CROSS REFERENCE TO RELATED APPLICATIONS

The presently disclosed subject matter claims the benefit of U.S. Provisional Patent Application Ser. No. 63/040,254, filed Jun. 17, 2020; U.S. Provisional Patent Application Ser. No. 63/073,234, filed Sep. 1, 2020; and U.S. Provisional Patent Application Ser. No. 63/129,145, filed Dec. 22, 2020; the disclosures of each of which are incorporated herein by reference in their entireties.

### GOVERNMENT SUPPORT

This invention was made with government support under Grant Nos. AI124214 and TR003015 awarded by the National Institutes of Health. The government has certain rights in the invention.

### REFERENCE TO SEQUENCE LISTING SUBMITTED ELECTRONICALLY

The content of the electronically submitted sequence listing in ASCII text file (Name: 3062-131_PCT_ST25.txt; Size: 54 kilobytes; and Date of Creation: Jun. 17, 2021) filed with the application is incorporated herein by reference in its entirety.

### TECHNICAL FIELD

The presently disclosed subject matter relates generally to methods for treating coronavirus disease 2019 (COVID-19), e.g., to reduce disease severity, and/or for predicting the need for mechanical ventilation in COVID-19 patients.

### BACKGROUND

Severe acute respiratory syndrome coronavirus 2 (SARS-CoV-2) is the infectious agent currently causing the global coronavirus disease 2019 (COVID-19) pandemic. SARS-CoV-2 primarily infects the lower respiratory tract of hosts by gaining entry to cells via the receptor angiotensin converting enzyme 2 (ACE2) facilitated by transmembrane receptor neuropilin-1 (Winkler et al., 2020a; and Hoffmann et al., 2020). The clinical course following infection varies widely, ranging from asymptomatic carriage to life-threatening respiratory failure and death.

Early on in the pandemic, it was recognized that patients with severe forms of COVID-19, e.g., hospitalization or ventilation, exhibited elevated levels of inflammatory cytokines (Pedersen and Ho, 2020). This inflammatory state can result in end-organ damage and, in some cases, death (Mangalmurti and Hunter, 2020; Tisoncik et al., 2012). While it was unclear how individual cytokines associated with this response are involved in severe outcomes, inflammation appeared to be a driver of the disease. In support of this hypothesis, the use of the anti-inflammatory steroid dexamethasone, was found to decrease mortality by 29% in COVID-19 patients requiring mechanical ventilation (The RECOVERY Collaborative Group, 2020).

However, there is an ongoing need for additional characterization of the host response to SARS-CoV-2 infection and for the more particular identification of contributors to severe clinical outcomes, e.g., to provide additional and more targeted therapeutic approaches to treating SARS-CoV-2 infections/COVID-19. There is also an ongoing need for additional methods of predicting disease severity, such as ventilation risk, in COVID-19 patients.

### SUMMARY

This Summary lists several embodiments of the presently disclosed subject matter, and in many cases lists variations and permutations of these embodiments of the presently disclosed subject matter. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

In some embodiments, the presently disclosed subject matter provides a method of treating coronavirus disease 2019 (COVID-19) in a subject in need thereof, the method comprising administering to the subject a composition that comprises a therapeutically effective amount of at least one agent that neutralizes one or more of interleukin-13 (IL-13), interleukin-6 (IL-6) and Janus kinase (JAK). In some embodiments, the composition comprises a therapeutically effective amount of at least one agent that neutralizes IL-13. In some embodiments, the at least one agent that neutralizes IL-13 also neutralizes interleukin-4 (IL-4). In some embodiments, the at least one agent is an antagonist of IL-4 receptor alpha 1 (IL4Rα1). In some embodiments, the at least one agent is an antibody that binds to IL4Rα1. In some embodiments, the at least one agent that neutralizes IL-13 is an antibody. In some embodiments, the antibody is a monoclonal antibody selected from the group comprising dupilumab, lebrikizumab, and tralokinumab. In some embodiments, the monoclonal antibody is dupilumab.

In some embodiments, the composition comprises a therapeutically effective amount of at least one agent that neutralizes IL-6. In some embodiments, the at least one agent is an antibody. In some embodiments, the antibody is a monoclonal antibody selected from the group comprising tocilizumab and sarilumab.

In some embodiments, the composition comprises a therapeutically effective amount of at least one agent that neutralizes JAK. In some embodiments, the at least one agent is a small molecule JAK inhibitor. In some embodiments, the small molecule JAK inhibitor is selected from the group comprising baricitinib and tofacitinib.

In some embodiments, treating COVID-19 reduces pulmonary inflammation. In some embodiments, treating COVID-19 reduces the risk of one or more of ventilation dependence, hospitalization, and death.

In some embodiments, the presently disclosed subject matter provides a method of predicting increased risk for mechanical ventilation in a subject having COVID-19, the method comprising: (a) measuring a plasma concentration of one or more cell signaling protein in the subject, wherein the one or more cell signaling protein comprises at least one selected from the group comprising IL-13, interleukin-7 (IL-7), and basic fibroblast growth factor (FGF-basic); (b) comparing the plasma concentration measured in the subject with a reference concentration of the one or more cell signaling protein; (c) predicting that the subject has an increased risk for mechanical ventilation when the plasma concentration measured in the subject is higher than the reference concentration or that the subject does not have an increased risk for mechanical ventilation when the plasma concentration measured in the subject is not higher than the reference concentration; and (d) administering to the subject a composition that comprises a therapeutically effective amount of at least one agent that neutralizes one or more of IL-13, IL-6 and JAK if the subject is predicted to have an increased risk of mechanical ventilation.

In some embodiments, step (a) comprises measuring a plasma concentration of IL-13. In some embodiments, step (a) further comprises measuring a plasma concentration of IL-6. In some embodiments, step (a) further comprises measuring a plasma concentration of interleukin-8 (IL-8) and macrophage inflammatory protein-1 beta (MIP-13).

Accordingly, it is an object of the presently disclosed subject matter to provide methods of treating COVID-19 and/or of predicting mechanical ventilation risk in subjects with COVID-19.

This and other objects are achieved in whole or in part by the presently disclosed subject matter. Further, objects of the presently disclosed subject matter having been stated above, other objects and advantages of the presently disclosed subject matter will become apparent to those skilled in the art after a study of the following description, Figures, and Examples. Additionally, various aspects and embodiments of the presently disclosed subject matter are described in further detail below.

### BRIEF DESCRIPTION OF THE FIGURES

FIGS. 1A-1D. Ventilation in COVID-19 patients is associated with inflammatory responses including interleukin 13 (IL-13), interleukin 7 (IL-7), and fibroblast growth factor-basic (bFGF). FIG. 1A is a graph showing hierarchical clustering of patients and cytokines done using pheatmap function in R. Cytokines are shown on the x-axis, and patients on the y-axis. Ventilation status is also shown on the left y-axis, with 0 being no ventilation and 1 being ventilation required. FIGS. 1B-1D are graphs of patient cytokine levels: IL-13 (FIG. 1B); IL-7 (FIG. 1C); and FGF-basic (FIG. 1D). Data in each of the graphs is divided into halves or quartiles and plotted in Kaplan-Meier curves for time to ventilation (in days after diagnosis). Odds ratios were calculated using logistic regression. P<0.05 was considered significant.

FIG. 2 is a series of graphs showing that ventilation in coronavirus disease 2019 (COVID-19) patients is associated with an increase in inflammatory cytokines. Levels (in picograms per milliliter (pg/ml)) of interleukin-7 (IL-7, top left), interleukin 13 (IL-13, top second from left), fibroblast growth factor (FGF)-basic (top second from right), interferon-gamma (IFN-g, top right), interleukin 17 (IL-17, bottom left), granulocyte colony stimulating factor (G-CSF, bottom, middle), and macrophage inflammatory protein-1 alpha (MIP-1α, bottom right) were measured from patient sera, and compared between patients who were or were not ventilated. Statistics were performed using a Mann-Whitney U test; p<0.05 was considered significant and is denoted by *. Ventilated: n=27. Not ventilated: n=30.

FIGS. 3A and 3B. Elevated glucose is associated with increased levels of interferon gamma (IFN-g) and interleukin-1 receptor agonist (IL-1ra). Biomarkers were compared between coronavirus disease 2019 (COVID-19) positive patients with elevated blood glucose (>126 milligrams per deciliter (mg/dl)) and patients with normal glucose (<125 mg/dl). IFN-g (FIG. 3A) and IL-1ra (FIG. 3B) were significantly elevated in patients with elevated glucose. Statistics were done using a Mann-Whitney U test; p<0.05 was considered significant and is denoted by *. Elevated glucose: n=19; Normal glucose: n=19.

FIGS. 4A and 4B. Mouse Model of coronavirus disease 2019 (COVID-19) Pneumonia. K18-hACE2 C57BI/6J mice were intranasally inoculated with 8×10⁴ fifty percent tissue culture infective dose (TCID50) of severe acute respiratory syndrome coronavirus 2 (SARS-CoV-2; Hong Kong/VM200001061/2020). FIG. 4A is a graph of the average percentage (%) of initial body weight in infected (circles) versus mock-infected (squares) mice as a function of time (in days) post-infection or mock-infection. FIG. 4B is a graph of clinical score (comprised of weight loss, eye closure, appearance of fur and posture, and respiration) in mice as a function of time (in days) post infection. The left-hand bar in each day's pair of bars corresponds to infected mice, while the right-hand bar corresponds to uninfected (mock-infected) mice.

FIGS. 5A-5C. Lung histology of mice infected with coronavirus disease 2019 (COVID-19). FIGS. 5A and 5B are representative microscope images from H&E sections of lung from K18-hACE2 C57BI6J mice, where FIG. 5A is an image from mice infected with severe acute respiratory syndrome coronavirus 2 (SARS-CoV-2) as described for FIGS. 4A and 4B, and FIG. 5B is an image from control (mock-infected) mice. Scale bars in the upper right-hand corner of each image represent 90 micrometers (m). FIG. 5C is a graph showing the histologic lung injury score (scored blinded) of the control (mock) mice (left) and the infected mice (right). **p<0.01.

FIGS. 6A-6C. Inflammatory cells in bronchoalveolar lavage (BAL) fluid from severe acute respiratory syndrome coronavirus 2 (SARS-CoV-2) infected K18-hACE2 C57BI/6J mice. Data is shown for mice infected with SARS-CoV-2 as described for FIGS. 4A and 4B (infected, right-hand bar in each graph) and mock-infected control mice (Mock, left-hand bar in each graph). Infected mice had more neutrophils (FIG. 6A), monocytes (FIG. 6B) and eosinophils (FIG. 6C) in BAL fluid. * p<0.05.

FIGS. 7A-7D. Interleukin 13 (IL-13) neutralization protects from weight loss and clinical score in severe acute respiratory syndrome coronavirus 2 (SARS-COV-2) infected K18-hACE2 C57BI/6J mice. Anti-IL13 monoclonal antibody (mAb) was administered intraperitoneally on days 0 and 2 of infection to K18-hACE2 C57BI/6J mice intranasally inoculated with 8×10⁴ fifty percent tissue culture infective dose (TCID50) of SARS-CoV-2 (Hong Kong/VM200001061/2020). FIG. 7A is a graph of weight loss (shown as a percentage (%) of starting weight as a function of time (days post infection (DPI), where black circles represent mice treated with control immunoglobulin G (IgG) and grey circles represent mice treated with anti-IL13 mAb. FIG. 7B is a graph of clinical score (comprised weight loss, eye closure, appearance of fur and posture, and respiration) in the same groups of mice as a function of DPI. Dark grey bars represent mice treated with control IgG, while lighter grey bars represent mice treated with anti-IL13 mAb. FIG. 7C is a graph of CD4, CD8, eosinophils, monocytes, and neutrophils as a percent of total cells in bronchoalveolar lavage (BAL) fluid cells analyzed by flow cytometry in infected mice treated with control IgG (dark grey bars) or anti-IL13 mAb (light grey bars). FIG. 7D is a graph of lung plaque forming units (PFU) in infected mice treated with control IgG and anti-IL13 mAb (aIL13). *p<0.05.

FIGS. 8A-8D. Interleukin-4 (IL-4) is increased in coronavirus disease 2019 (COVID-19) inpatients who require mechanical ventilation. FIG. 8A is a graph of plasma IL-4 (measured in picograms per milliliter (pg/ml)) in a cohort of 70 COVID-19 patients using a 47 cytokine assay where results are separated by patient ventilation status (ventilated versus not ventilated). FIG. 8B is a graph of plasma interleukin 13 (IL-13) (measured in pg/ml) in a cohort of 70 COVID-19 patients using a 47 cytokine assay where results are separated by patient ventilation status (ventilated versus not ventilated). FIG. 8C is a graph of plasma IL-4 (measured in pg/ml) in a cohort of 70 COVID-19 patients using a 47 cytokine assay where the results are separated by hospitalization status (outpatient versus inpatient). FIG. 8D is a graph of plasma IL-13 (measured in pg/ml) in a cohort of 70 COVID-19 patients using a 47 cytokine assay where the results are separated by hospitalization status (outpatient versus inpatient).

FIGS. 9A-9H. Plasma interleukin 4 (IL-4) and interleukin 13 (IL-13) levels determined in different cohort subsets, i.e., an “overlap” subset of patients that are in both the 70 patient cohort described for FIGS. 8A-8D and in the smaller cohort of patients used in the study described for FIG. 2, and a “unique patient” subset of patients in only the cohort described for FIGS. 8A-8D. FIG. 9A is a graph of plasma IL-4 (measured in picograms per milliliter (pg/ml)) in the overlap subset of COVID-19 patients where results are separated by patient ventilation status (ventilated versus not ventilated). FIG. 9B is a graph of IL-13 (measured in pg/ml) in the overlap subset of COVID-19 patients separated by patient ventilation status (ventilated versus not ventilated). FIG. 9C is a graph of plasma IL-4 (measured in pg/ml) in the unique patient subset separated by ventilation status (ventilated versus not ventilated). FIG. 9D is a graph of plasma IL-13 (measured in pg/ml) in the unique patient subset separated by ventilation status (ventilated versus not ventilated). FIG. 9E is a graph of plasma IL-4 (measured in pg/ml) in the overlap subset with results separated by hospitalization status (outpatient versus inpatient). FIG. 9F is a graph of plasma IL-13 (measured in pg/ml) in the overlap subset where the results are separated by hospitalization status (outpatient versus inpatient). FIG. 9G is a graph of plasma IL-4 (measured in pg/ml) in the unique patient subset with results separated by hospitalization status (outpatient versus inpatient). FIG. 9H is a graph of plasma IL-13 (measured in pg/ml) in the unique patient subset where the results are separated by hospitalization status (outpatient versus inpatient).

FIGS. 10A-10F. Type 2 immune response in patients with severe coronavirus disease 2019 (COVID-19) disease. Cytokines were measured in plasma from 26 outpatients and 152 inpatients with COVID-19 infection at the University of Virginia Hospital using a 48-plex cytokine array. FIG. 10A is a heatmap of plasma cytokines, supplemental oxygen requirement and nasopharyngeal viral load, with rows ordered by patient status (outpatient (OP) vs inpatient (IP)) and columns by cytokine principal component 1 which included interleukin 13 (IL-13). FIG. 10B is a graph showing a scatterplot comparing principal component 1 and 2 from Principal Component Analysis (PCA) of the plasma cytokines (grey=inpatients; black=outpatients). FIG. 10C is a graph of plasma IL-13 levels in (picograms per milliliter (pg/ml)) in patients who were or were not diagnosed with COVID-19. FIG. 10D is a graph of plasma IL-13 levels (in pg/ml) in COVID-19 patients who did or did not require mechanical ventilation (Wilcox test). FIG. 10E is a graph of Kaplan-Meier survival analysis of the relationship between IL-13 level and mechanical ventilation. Comparison made to lowest IL-13 quantile (Cox proportional hazards test adjusted for age, sex, and comorbidities; CI=confidence interval). FIG. 10F is a graph showing the proportion of COVID-19 patients requiring mechanical ventilation (light grey) versus not requiring mechanical ventilation (dark grey) stratified by IL-13 μlasma cytokine levels (Chi-square analysis). FIG. 10G is a ROC curve with AUC plotted from: IL-13 alone, IL-13 and IL-6, or IL-13, IL-6, IL-8, and MIP-1b. FIG. 10H is a graph of IL-13 μlasma levels (in pg/ml) in non-severe and severe (requiring supplemental oxygen) COVID-19 patients from Virginia Commonwealth University Hospital (Wilcox test). *=p<0.05; 9 **=p<0.005.

FIG. 11. A schematic diagram showing network analysis of cytokine genes in patients with coronavirus disease 2019 (COVID-19). The network analysis captures the structural relationship among cytokine measurements with graphical least absolute shrinkage and solution operator (LASSO) analysis. The nodes represent individual cytokines and edges represent their correlations in that highly correlated cytokines are connected closer with thick edges.

FIGS. 12A-12C. Type 2 immune response in lungs of mice following infection with severe acute respiratory syndrome coronavirus 2 (SARS-CoV-2). 10-week-old male mice (Tg K18-hACE2 2Prlmn) were infected with 5×10³ μlaque forming units (PFU) of SARS-CoV-3 and lung tissue examined on day five post-infection by RNA-seq and immunohistochemistry (IHC). FIG. 12A is a heatmap of type 2 gene expression in the lungs of infected vs uninfected mice (heat map of normalized values of manually curated list of type 2 immune pathway genes). FIG. 12B is a pair of images of immunohistochemistry of the type 2 immunity proteins resistin-like molecule alpha (RELMa) and chitinase-like protein 3 (Ym1) in the lungs of infected (bottom) and uninfected (top) mice (AW, airway). FIG. 12C is a series of graphs of the quantification of IHC scoring for RELMa (parenchyma (middle graph) and epithelial (right graph)) and Ym1 (left graph) (mixed effect model) in infected and non-infected mice. IntDen=Integrated Density; BM=Basement membrane. Scale bar=70 micrometers (μm). *=p<0.05; **=p<0.005.

FIGS. 13A-13F. Interleukin 13 (IL-13) neutralization protects from severe coronavirus disease 2019 (COVID-19) in K18-hACE2 mice. Mice were infected on day 0 with 5×10³ μlaque forming units (PFU) of severe acute respiratory syndrome coronavirus 2 (SARS-CoV-2) and administered 150 micrograms (pg) of anti-IL-13 monoclonal antibody (aIL-13) or an isotope control antibody (immunoglobulin G (IgG)) intraperitoneally on days 0, 2, and 4. FIG. 13A is a graph of clinical scores of illness severity on days 1-7 post-infection in SARS-CoV-2 infected mice treated with aIL-13 (light grey bars) or IgG (dark grey bars). Clinical scoring was measured by weight loss (0-5), posture and 5 appearance of fur (piloerection) (0-2), activity (0-3) and eye closure (0-2). FIG. 13B is a graph of weight loss (measured as a percentage (%) of starting weight on days 1-7 post-infection in SARS-CoV-2 infected mice treated with aIL-13 (light grey bars) or IgG (dark grey bars). FIG. 13C is a graph of Kaplan-Meier survival analysis in SARS-CoV-2 mice treated with aIL-13 or IgG. FIG. 13D is a graph of Kaplan-Meier curves generated from data obtained from an international patient cohort with 1:1 matching based on 81 patients who had been prescribed Dupilumab independently of their COVID-19 diagnosis versus those who had not (control). FIG. 13E is a pair of graphs of the quantification of intensity of staining for parenchyma (left graph) and epithelial (right graph) RELMa following IL-13 neutralization (log transformed, mixed effect model). FIG. 13F is a pair of images of immunohistochemistry of lung tissue stained for RELMα (light color) in parenchyma or airway, and DAPI in infected mice treated with control (IgG, top) or anti-IL-13 mAb (bottom). (N=5 mice/group; the data in FIGS. 13A and 13B are combined from three independently conducted experiments). *=p<0.05; **=p<0.005

FIGS. 14A-14G. Impact of anti-interleukin 13 antibody (anti-IL-13) on lung injury and inflammation in a mouse model of coronavirus disease 2019 (COVID-19). Mice were infected with 5×10³ μlaque forming units (PFU) of severe acute respiratory syndrome coronavirus 2 (SARS-CoV-2) on day 0 and given 150 micrograms (pg) of anti-IL-13 or an isotype control antibody (immunoglobulin G (IgG)) on days 0, 2, and 4. On day five, mice were euthanized and bronchoalveolar lavage (BAL) fluid collected. For histology, lungs were inflated with formalin before removing and fixing prior to H&E staining. FIG. 14A is a graph of viral burden in lungs on day 5 post-infection measured by PFU. FIG. 14B is a pair of microscope images of Hematoxylin and eosin (H&E) staining of infected mouse lung with or without anti-IL-13. FIG. 14C is a graph of the quantified lung injury score of the mouse lung samples described for FIG. 14B. FIG. 14D is a graph of goblet cells quantified from periodic acid-Schiff (PAS) staining of lung tissue from day five post-infection. FIG. 14E is a graph of chitinase-like protein 3 (Ym1) integrated density (IntDen) with or without anti-IL-13. FIG. 14F is a heatmap of cytokines in BAL measured by Luminex (plotted with group identity and clinical score). FIG. 14G is a graph of immune cells in BAL quantified by flow cytometry. NS=not significant; ***=p<0.01

### DETAILED DESCRIPTION

The presently disclosed subject matter will now be described more fully. The presently disclosed subject matter can, however, be embodied in different forms and should not be construed as limited to the embodiments set forth herein below and in the accompanying Examples. Rather, these embodiments are provided so that this disclosure will be thorough and complete, and will fully convey the scope of the embodiments to those skilled in the art.

All references listed herein, including but not limited to all patents, patent applications and publications thereof, and scientific journal articles, are incorporated herein by reference in their entireties to the extent that they supplement, explain, provide a background for, or teach methodology, techniques, and/or compositions employed herein.

### I. Definitions

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the presently disclosed and claimed subject matter.

Following long-standing patent law convention, the terms “a”, “an”, and “the” refer to “one or more” when used in this application, including in the claims. For example, the phrase “a therapeutic agent” refers to one or more therapeutic agents, e.g., one or more of the same of different therapeutic agents. Similarly, the phrase “at least one”, when employed herein to refer to an entity, refers to, for example, 1, 2, 3, 4, 5, 6, 7, 8, 9, 10, 15, 20, 25, 30, 35, 40, 45, 50, 75, 100, or more of that entity, including but not limited to whole number values between 1 and 100 and greater than 100.

Unless otherwise indicated, all numbers expressing quantities of time, concentration percent inhibition, percent viability, amounts of ingredients, reaction conditions, and so forth used in the specification and claims are to be understood as being modified in all instances by the term “about”. The term “about”, as used herein when referring to a measurable value such as an amount of mass, weight, time, volume, concentration, or percentage, is meant to encompass variations of in some embodiments±20%, in some embodiments±10%, in some embodiments±5%, in some embodiments±1%, in some embodiments±0.5%, and in some embodiments±0.1% from the specified amount, as such variations are appropriate to perform the disclosed methods and/or employ the disclosed compositions. Accordingly, unless indicated to the contrary, the numerical parameters set forth in this specification and attached claims are approximations that can vary depending upon the desired properties sought to be obtained by the presently disclosed subject matter.

Numerical ranges recited herein by endpoints include all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about” unless stated otherwise.

A disease or disorder is “alleviated” if the severity of a symptom of the disease, condition, or disorder, or the frequency at which such a symptom is experienced by a subject, or both, are reduced.

As used herein, the term “and/or” when used in the context of a list of entities, refers to the entities being present singly or in combination. Thus, for example, the phrase “A, B, C, and/or D” includes A, B, C, and D individually, but also includes any and all combinations and subcombinations of A, B, C, and D.

The terms “additional therapeutically active compound” and “additional therapeutic agent”, as used in the context of the presently disclosed subject matter, refers to the use or administration of a compound for an additional therapeutic use for a particular injury, disease, or disorder being treated. Such a compound, for example, could include one being used to treat an unrelated disease or disorder, or a disease or disorder which may not be responsive to the primary treatment for the injury, disease, or disorder being treated.

As used herein, the term “adjuvant” refers to a substance that elicits an enhanced immune response when used in combination with a specific antigen.

As use herein, the terms “administration of” and/or “administering” a compound should be understood to refer to providing a compound of the presently disclosed subject matter to a subject in need of treatment.

The term “comprising”, which is synonymous with “including” “containing”, or “characterized by”, is inclusive or open-ended and does not exclude additional, unrecited elements and/or method steps. “Comprising” is a term of art that means that the named elements and/or steps are present, but that other elements and/or steps can be added and still fall within the scope of the relevant subject matter.

As used herein, the phrase “consisting essentially of” limits the scope of the related disclosure or claim to the specified materials and/or steps, plus those that do not materially affect the basic and novel characteristic(s) of the disclosed and/or claimed subject matter. For example, a pharmaceutical composition can “consist essentially of” a pharmaceutically active agent or a plurality of pharmaceutically active agents, which means that the recited pharmaceutically active agent(s) is/are the only pharmaceutically active agent(s) present in the pharmaceutical composition. It is noted, however, that carriers, excipients, and/or other inactive agents can and likely would be present in such a pharmaceutical composition and are encompassed within the nature of the phrase “consisting essentially of”.

As used herein, the phrase “consisting of” excludes any element, step, or ingredient not specifically recited. It is noted that, when the phrase “consists of” appears in a clause of the body of a claim, rather than immediately following the preamble, it limits only the element set forth in that clause; other elements are not excluded from the claim as a whole.

With respect to the terms “comprising”, “consisting of”, and “consisting essentially of”, where one of these three terms is used herein, the presently disclosed and claimed subject matter can include the use of either of the other two terms. For example, a composition that in some embodiments comprises a given active agent also in some embodiments can consist essentially of that same active agent, and indeed can in some embodiments consist of that same active agent.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin subunit molecules. The antibodies in the presently disclosed subject matter can exist in a variety of forms including, for example, polyclonal antibodies, monoclonal antibodies, Fv, Fab and F(ab)₂, as well as single chain antibodies and humanized antibodies.

An “antibody heavy chain,” as used herein, refers to the larger of the two types of polypeptide chains present in all antibody molecules.

An “antibody light chain,” as used herein, refers to the smaller of the two types of polypeptide chains present in all antibody molecules.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

As used herein, the term “secondary antibody” refers to an antibody that binds to the constant region of another antibody (the primary antibody).

The term “antigen” as used herein is defined as a molecule that provokes an immune response. This immune response can involve either antibody production, or the activation of specific immunologically-competent cells, or both. An antigen can be derived from organisms, subunits of proteins/antigens, killed or inactivated whole cells or lysates.

The term “antigenic determinant” as used herein refers to that portion of an antigen that makes contact with a particular antibody (i.e., an epitope). When a protein or fragment of a protein, or chemical moiety is used to immunize a host animal, numerous regions of the antigen can induce the production of antibodies that bind specifically to a given region or three-dimensional structure on the protein; these regions or structures are referred to as antigenic determinants. An antigenic determinant can compete with the intact antigen (i.e., the “immunogen” used to elicit the immune response) for binding to an antibody.

The term “antimicrobial agents” as used herein refers to any naturally-occurring, synthetic, or semi-synthetic compound or composition or mixture thereof, which is safe for human or animal use as practiced in the methods of the presently disclosed subject matter, and is effective in killing or substantially inhibiting the growth of microbes. “Antimicrobial” as used herein, includes antibacterial, antifungal, and antiviral agents.

A pathology or symptom “associated” with C. difficile infection refers to mortality, colonic inflammation, diarrhea, weight loss, changes in expression and levels of genes, proteins, and cells as described herein or those that are known in the art that occur upon the infection or are a result of the infection.

The term “aqueous solution” as used herein can include other ingredients commonly used, such as sodium bicarbonate described herein, and further includes any acid or base solution used to adjust the pH of the aqueous solution while solubilizing a peptide.

The term “binding” refers to the adherence of molecules to one another, such as, but not limited to, enzymes to substrates, ligands to receptors, antibodies to antigens, DNA binding domains of proteins to DNA, and DNA or RNA strands to complementary strands.

“Binding partner”, as used herein, refers to a molecule capable of binding to another molecule.

The term “biocompatible”, as used herein, refers to a material that does not elicit a substantial detrimental response in the host.

As used herein, the terms “biologically active fragment” and “bioactive fragment” of a peptide encompass natural and synthetic portions of a longer peptide or protein that are capable of specific binding to their natural ligand and/or of performing a desired function of a protein, for example, a fragment of a protein of larger peptide which still contains the epitope of interest and is immunogenic.

The term “biological sample”, as used herein, refers to samples obtained from a subject, including but not limited to skin, hair, tissue, blood, plasma, cells, sweat, and urine.

As used herein, the term “conservative amino acid substitution” is defined herein as an amino acid exchange within one of the following five groups: I) Small aliphatic, nonpolar or slightly polar residues: Ala, Ser, Thr, Pro, Gly; II) Polar, negatively charged residues and their amides: Asp, Asn, Glu, Gln; III) Polar, positively charged residues: His, Arg, Lys; IV) Large, aliphatic, nonpolar residues: Met Leu, Ile, Val, Cys; and V) Large, aromatic residues: Phe, Tyr, Trp.

“Cytokine,” as used herein, refers to intercellular signaling molecules, the best known of which are involved in the regulation of mammalian somatic cells. A number of families of cytokines, both growth promoting and growth inhibitory in their effects, have been characterized including, for example, interleukins, interferons, and transforming growth factors. A number of other cytokines are known to those of skill in the art. The sources, characteristics, targets, and effector activities of these cytokines have been described.

A “coding region” of a gene comprises the nucleotide residues of the coding strand of the gene and the nucleotides of the non-coding strand of the gene which are homologous with or complementary to, respectively, the coding region of an mRNA molecule which is produced by transcription of the gene.

“Complementary” as used herein refers to the broad concept of subunit sequence complementarity between two nucleic acids (e.g., two DNA molecules). When a nucleotide position in both of the molecules is occupied by nucleotides normally capable of base pairing with each other at a given position, the nucleic acids are considered to be complementary to each other at this position. Thus, two nucleic acids are complementary to each other when a substantial number (in some embodiments at least 50%) of corresponding positions in each of the molecules are occupied by nucleotides that can base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. By way of example and not limitation, the first region comprises a first portion and the second region comprises a second portion, whereby, when the first and second portions are arranged in an antiparallel fashion, in some embodiments at least about 50%, in some embodiments at least about 75%, in some embodiments at least about 90%, and in some embodiments at least about 95% of the nucleotide residues of the first portion are capable of base pairing with nucleotide residues in the second portion. In some embodiments, all nucleotide residues of the first portion are capable of base pairing with nucleotide residues in the second portion.

“Co-administer” can include simultaneous and/or sequential administration of two or more agents.

A “compound,” as used herein, refers to any type of substance or agent that is can be considered a drug, or a candidate for use as a drug, as well as combinations and mixtures of the above.

A “control” cell, tissue, sample, or subject is a cell, tissue, sample, or subject of the same type as a test cell, tissue, sample, or subject. The control can, for example, be examined at precisely or nearly the same time the test cell, tissue, sample, or subject is examined. The control can also, for example, be examined at a time distant from the time at which the test cell, tissue, sample, or subject is examined, and the results of the examination of the control can be recorded so that the recorded results can be compared with results obtained by examination of a test cell, tissue, sample, or subject. The control can also be obtained from another source or similar source other than the test group or a test subject, where the test sample is obtained from a subject suspected of having a condition, disease, or disorder for which the test is being performed.

A “test” cell is a cell being examined.

A “pathoindicative” cell is a cell that, when present in a tissue, is an indication that the animal in which the tissue is located (or from which the tissue was obtained) is afflicted with a condition, disease, or disorder.

A “pathogenic” cell is a cell that, when present in a tissue, causes or contributes to a condition, disease, or disorder in the animal in which the tissue is located (or from which the tissue was obtained).

A tissue “normally comprises” a cell if one or more of the cell are present in the tissue in an animal not afflicted with a condition, disease, or disorder.

As used herein, a “derivative” of a compound refers to a chemical compound that can be produced from another compound of similar structure in one or more steps, as in replacement of H by an alkyl, acyl, or amino group.

The use of the word “detect” and its grammatical variants refers to measurement of the species without quantification, whereas use of the word “determine” or “measure” with their grammatical variants are meant to refer to measurement of the species with quantification. The terms “detect” and “identify” are used interchangeably herein.

As used herein, the terms “condition”, “disease condition”, “disease”, “disease state”, and “disorder” refer to physiological states in which diseased cells or cells of interest can be targeted with the compositions of the presently disclosed subject matter.

As used herein, the term “diagnosis” refers to detecting a risk or propensity to a condition, disease, or disorder. In any method of diagnosis exist false positives and false negatives. Any one method of diagnosis does not provide 100% accuracy.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, an “effective amount” or “therapeutically effective amount” refers to an amount of a compound or composition sufficient to produce a selected effect, such as but not limited to alleviating symptoms of a condition, disease, or disorder. In the context of administering compounds in the form of a combination, such as multiple compounds, the amount of each compound, when administered in combination with one or more other compounds, can be different from when that compound is administered alone. Thus, an effective amount of a combination of compounds refers collectively to the combination as a whole, although the actual amounts of each compound can vary. The term “more effective” means that the selected effect occurs to a greater extent by one treatment relative to the second treatment to which it is being compared.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA, and mRNA) or a defined sequence of amino acids and the biological properties resulting therefrom. Thus, a gene encodes a protein if transcription and translation of an mRNA corresponding to or derived from that gene produces the protein in a cell or other biological system and/or an in vitro or ex vivo system. Both the coding strand, the nucleotide sequence of which is identical to the mRNA sequence (with the exception of uracil bases presented in the latter) and is usually provided in Sequence Listing, and the non-coding strand, used as the template for transcription of a gene or cDNA, can be referred to as encoding the protein or other product of that gene or cDNA.

As used herein, an “essentially pure” preparation of a particular protein or peptide is a preparation wherein in some embodiments at least about 95% and in some embodiments at least about 99%, by weight, of the protein or peptide in the preparation is the particular protein or peptide.

A “fragment” or “segment” or “subsequence” is a portion of an amino acid sequence comprising at least one amino acid or a portion of a nucleic acid sequence comprising at least one nucleotide. The terms “fragment” and “segment” are used interchangeably herein.

As used herein, the term “fragment,” as applied to a protein or peptide, can ordinarily be at least about 3-15 amino acids in length, at least about 15-25 amino acids, at least about 25-50 amino acids in length, at least about 50-75 amino acids in length, at least about 75-100 amino acids in length, and greater than 100 amino acids in length.

As used herein, the term “fragment” as applied to a nucleic acid, can ordinarily be at least about 20 nucleotides in length, typically, at least about 50 nucleotides, more typically, from about 50 to about 100 nucleotides, at least about 100 to about 200 nucleotides, at least about 200 nucleotides to about 300 nucleotides, at least about 300 to about 350, at least about 350 nucleotides to about 500 nucleotides, at least about 500 to about 600, at least about 600 nucleotides to about 620 nucleotides, at least about 620 to about 650, and or the nucleic acid fragment will be greater than about 650 nucleotides in length.

As used herein, a “functional” biological molecule is a biological molecule in a form in which it exhibits a property by which it can be characterized. A functional enzyme, for example, is one that exhibits the characteristic catalytic activity by which the enzyme can be characterized.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC share 50% homology.

As used herein, “homology” is used synonymously with “identity.”

The determination of percent identity between two nucleotide or amino acid sequences can be accomplished using a mathematical algorithm. For example, a mathematical algorithm useful for comparing two sequences is the algorithm incorporated into the NBLAST and XBLAST programs that can be accessed, for example, at the National Center for Biotechnology Information (NCBI) world wide web site. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized. Alternatively, PSI-Blast or PHI-Blast can be used to perform an iterated search which detects distant relationships between molecules and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

As used herein “injecting”, “applying”, and “administering” include administration of a compound of the presently disclosed subject matter by any number of routes and modes including, but not limited to, topical, oral, buccal, intravenous, intramuscular, intra-arterial, intramedullary, intrathecal, intraventricular, transdermal, subcutaneous, intraperitoneal, intranasal, enteral, topical, sublingual, vaginal, ophthalmic, pulmonary, vaginal, and rectal approaches.

As used herein, a “ligand” is a compound that specifically binds to a target compound or molecule. A ligand “specifically binds to” or “is specifically reactive with” a compound when the ligand functions in a binding reaction which is determinative of the presence of the compound in a sample of heterogeneous compounds.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, such as but not limited to through ionic or hydrogen bonds or van der Waals interactions.

As used herein, the term “mammal” refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

The terms “measuring the level of expression” and “determining the level of expression” as used herein refer to any measure or assay which can be used to correlate the results of the assay with the level of expression of a gene or protein of interest. Such assays include measuring the level of mRNA, protein levels, etc. and can be performed by assays such as northern and western blot analyses, binding assays, immunoblots, etc. The level of expression can include rates of expression and can be measured in terms of the actual amount of an mRNA or protein present. Such assays are coupled with processes or systems to store and process information and to help quantify levels, signals, etc. and to digitize the information for use in comparing levels.

The term “otherwise identical sample”, as used herein, refers to a sample similar to a first sample, that is, it is obtained in the same manner from the same subject from the same tissue or fluid, or it refers a similar sample obtained from a different subject. The term “otherwise identical sample from an unaffected subject” refers to a sample obtained from a subject not known to have the disease or disorder being examined. The sample can of course be a standard sample. By analogy, the term “otherwise identical” can also be used regarding regions or tissues in a subject or in an unaffected subject.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

The term “pharmaceutical composition” refers to a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

“Pharmaceutically acceptable” means physiologically tolerable, for either human or veterinary application. Similarly, “pharmaceutical compositions” include formulations for human and veterinary use.

As used herein, the term “pharmaceutically acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Plurality” means at least two.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” refers to non-naturally occurring peptides or polypeptides. Synthetic peptides or polypeptides can be synthesized, for example, using an automated polypeptide synthesizer. Various solid phase peptide synthesis methods are known to those of skill in the art.

The term “prevent”, as used herein, means to stop something from happening, or taking advance measures against something possible or probable from happening. In the context of medicine, “prevention” generally refers to action taken to decrease the chance of getting a disease or condition. It is noted that “prevention” need not be absolute, and thus can occur as a matter of degree.

In some embodiments, a “preventive” or “prophylactic” treatment is a treatment administered to a subject who does not exhibit signs, or exhibits only early signs, of a condition, disease, or disorder. Thus, a prophylactic or preventative treatment can be administered for the purpose of decreasing the risk of developing pathology associated with developing the condition, disease, or disorder.

The term “protein” typically refers to large polypeptides. Conventional notation is used herein to portray polypeptide sequences: the left-hand end of a polypeptide sequence is the amino-terminus; the right-hand end of a polypeptide sequence is the carboxyl-terminus.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process.

A “highly purified” compound as used herein refers to a compound that is in some embodiments greater than 90% pure, that is in some embodiments greater than 95% pure, and that is in some embodiments greater than 98% pure.

The term “subject” as used herein refers to a member of species for which treatment and/or prevention of a disease or disorder using the compositions and methods of the presently disclosed subject matter might be desirable. Accordingly, the term “subject” is intended to encompass in some embodiments any member of the Kingdom Animalia including, but not limited to the phylum Chordata (e.g., members of Classes Osteichythyes (bony fish), Amphibia (amphibians), Reptilia (reptiles), Aves (birds), and Mammalia (mammals), and all Orders and Families encompassed therein.

The compositions and methods of the presently disclosed subject matter are particularly useful for warm-blooded vertebrates. Thus, in some embodiments the presently disclosed subject matter concerns mammals and birds. More particularly provided are compositions and methods derived from and/or for use in mammals such as humans and other primates, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), rodents (such as mice, rats, and rabbits), marsupials, and horses. Also provided is the use of the disclosed methods and compositions on birds, including those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, also provided is the use of the disclosed methods and compositions on livestock, including but not limited to domesticated swine (pigs and hogs), ruminants, horses, poultry, and the like.

A “sample”, as used herein, refers in some embodiments to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

The term “specific binding” refers to a direct association between two molecules, due to, for example, covalent, electrostatic, hydrophobic, and ionic and/or hydrogen-bond interactions, including interactions such as salt bridges and water bridges. A specific binding member describes a member of a pair of molecules which have binding specificity for one another. The members of a specific binding pair may be naturally derived or wholly or partially synthetically produced. One member of the pair of molecules has an area on its surface, or a cavity, which specifically binds to and is therefore complementary to a particular spatial and polar organization of the other member of the pair of molecules. Thus, the members of the pair have the property of binding specifically to each other. Examples of pairs of specific binding members are antigen-antibody, biotin-avidin, hormone-hormone receptor, receptor-ligand, enzyme-substrate. Specific binding members of a binding pair exhibit high affinity and binding specificity for binding with the each other. Typically, affinity between the specific binding members of a pair is characterized by a K_{d} (dissociation constant) of 10⁻⁶ M or less, such as 10⁻⁷ M or less, including 10⁻⁸ M or less, e.g., 10⁻⁹ M or less, 10⁻¹⁰ M or less, 10⁻¹¹ M or less, 10⁻¹² M or less, 10⁻¹³ M or less, 10⁻¹⁴ M or less, including 10⁻¹⁵ M or less.

The term “standard”, as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

As used herein, a “subject in need thereof” is a patient, animal, mammal, or human, who will benefit from the method of the presently disclosed subject matter.

The term “substantially pure” describes a compound, e.g., a protein or polypeptide, cell or nucleic acid that has been separated from components which naturally accompany it. Typically, a compound is substantially pure when at least 10%, including at least 20%, at least 50%, at least 60%, at least 75%, at least 90%, at least 95%, at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

As used herein, a “substantially homologous amino acid sequences” or “substantially identical amino acid sequences” includes those amino acid sequences which have at least about 92%, or at least about 95% homology or identity, including at least about 96% homology or identity, including at least about 97% homology or identity, including at least about 98% homology or identity, and at least about 99% or more homology or identity to an amino acid sequence of a reference antibody chain. Amino acid sequence similarity or identity can be computed by using the BLASTP and TBLASTN programs which employ the BLAST (basic local alignment search tool) 2.0.14 algorithm. The default settings used for these programs are suitable for identifying substantially similar amino acid sequences for purposes of the presently disclosed subject matter.

“Substantially homologous nucleic acid sequence” or “substantially identical nucleic acid sequence” means a nucleic acid sequence corresponding to a reference nucleic acid sequence wherein the corresponding sequence encodes a peptide having substantially the same structure and function as the peptide encoded by the reference nucleic acid sequence; e.g., where only changes in amino acids not significantly affecting the peptide function occur. In one embodiment, the substantially identical nucleic acid sequence encodes the peptide encoded by the reference nucleic acid sequence. The percentage of identity between the substantially similar nucleic acid sequence and the reference nucleic acid sequence is at least about 50%, 65%, 75%, 85%, 92%, 95%, 99% or more. Substantial identity of nucleic acid sequences can be determined by comparing the sequence identity of two sequences, for example by physical/chemical methods (i.e., hybridization) or by sequence alignment via computer algorithm.

Suitable nucleic acid hybridization conditions to determine if a nucleotide sequence is substantially similar to a reference nucleotide sequence are: 7% sodium dodecyl sulfate SDS, 0.5 M NaPO₄, 1 mM EDTA at 50° C. with washing in 2×standard saline citrate (SSC), 0.1% SDS at 50° C.; preferably in 7% (SDS), 0.5 M NaPO₄, 1 mM EDTA at 50° C., with washing in 1×SSC, 0.1% SDS at 50° C.; preferably 7% SDS, 0.5 M NaPO₄, 1 mM EDTA at 50° C. with washing in 0.5 SSC, 0.1% SDS at 50° C.; and more preferably in 7% SDS, 0.5 M NaPO₄, 1 mM EDTA at 50° C. with washing in 0.1×SSC, 0.1% SDS at 65° C. Suitable computer algorithms to determine substantial similarity between two nucleic acid sequences include, GCS program package. The default settings provided with these programs are suitable for determining substantial similarity of nucleic acid sequences for purposes of the presently disclosed subject matter.

The term “substantially pure” describes a compound, e.g., a protein or polypeptide, which has been separated from components which naturally accompany it. Typically, a compound is substantially pure when in some embodiments at least 10%, in some embodiments at least 20%, in some embodiments at least 50%, in some embodiments at least 60%, in some embodiments at least 75%, in some embodiments at least 90%, and in some embodiments at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

The term “symptom”, as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse, and other observers.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

As used herein, the phrase “therapeutic agent” refers to an agent that is used to, for example, treat, inhibit, prevent, mitigate the effects of, reduce the severity of, reduce the likelihood of developing, slow the progression of, and/or cure, a disease or disorder.

The terms “treatment” and “treating” as used herein refer to both therapeutic treatment and prophylactic or preventative measures, wherein the object is to prevent or slow down (lessen) the targeted pathologic condition, prevent the pathologic condition, pursue or obtain beneficial results, and/or lower the chances of the individual developing a condition, disease, or disorder, even if the treatment is ultimately unsuccessful. Those in need of treatment include those already with the condition as well as those prone to have or predisposed to having a condition, disease, or disorder, or those in whom the condition is to be prevented. The term “treating” refers any effect, e.g., lessening, reducing, modulating, ameliorating, reversing or eliminating, that results in the improvement of the condition, disease, disorder, and the like, or ameliorating a symptom thereof.

As used herein, the terms “vector”, “cloning vector”, and “expression vector” refer to a vehicle by which a polynucleotide sequence (e.g., a foreign gene) can be introduced into a host cell, so as to transduce and/or transform the host cell in order to promote expression (e.g., transcription and translation) of the introduced sequence. Vectors include plasmids, phages, viruses, etc.

All genes, gene names, and gene products disclosed herein are intended to correspond to homologs and/or orthologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates.

### II. General Considerations

SARS-CoV-2 is a human coronavirus currently causing the global pandemic COVID-19. Complications of COVID-19, the disease caused by SARS-CoV-2, include pneumonia and acute respiratory distress syndrome. Severe disease also leads to excessive production of cytokines, commonly referred to as cytokine storm, and ultimately can result in significant lung damage. Cytokine storm, or cytokine release syndrome (CRS) is often characterized by high levels of IL-6, TNFa, IL-1b, and IL-10, which have all been observed to be elevated in patients with severe disease (Liu et al., 2020; Huang et al., 2020; Chen et al., 2020). Complications due to cytokine storm include shock and multiple organ failure and can be life-threatening. Additionally, patients with comorbidities can also have worse disease outcomes due to cytokine storm (Zaim et al. 2020). People with diabetes mellitus can have chronic elevated proinflammatory cytokines, causing them to be at increased risk of hyperinflammation of respiratory tissues and more severe disease due to COVID-19 (Guo et al. 2020, Bornstein et al. 2020, Singh et al. 2020). The presence of CRS in severe COVID-19 highlights the nature of immune dysregulation SARS-CoV-2 can induce and suggests an immunopathological response potentially targetable by therapeutics.

The presently disclosed subject matter is based in part on studies described herein showing correlation between mechanical ventilation and plasma cytokine levels observed in COVID-19 patient cohorts. In an initial COVID-19 patient cohort, hierarchical clustering, Kaplan-Meier curves, and odds ratios indicated that three cytokines, i.e., IL-13 (OR: 1.57), IL-7 (OR:1.04), and bFGF (or FGF-basic; OR:1.04), were predictive of ventilation in patients. Accordingly, the study suggested that elevations in cytokines in the type 2 or fibrosis pathways can be indicative of severe disease outcomes, e.g., hospitalization, ventilation, and death. The study also suggested that therapeutic manipulation (e.g., neutralization) of IL-13, IL-7 and FGF-basic could prevent severe disease outcomes, such as ventilator dependence. In addition, IFN-γ and IL-1ra elevation was observed in COVID-19 patients with diabetes mellitus, which suggested the manipulation (e.g., neutralization) of IFN-γ and IL-1ra in diabetic COVID-19 patients to improve disease outcome. Further, clustering of patients with both elevated IL-6 and IL-suggested that manipulation of either or both of these cytokines could be beneficial in COVID-19.

Given the elevated levels of IL-13 in COVID-19 patients, the role of IL-13 was investigated more closely. IL-13 is generally associated, for example with Type 2 inflammation. Type 2 inflammation is central to atopic diseases including asthma, allergic rhinitis, eosinophilic esophagitis and atopic dermatitis. In asthma, Type 2 inflammation in the lungs is characterized by the production of the cytokines IL-4, IL-13 and IL-5, and the presence in the airway spaces of infiltrating eosinophiis, lynphocytes (CD4 Th2 cells and IgE producing B cells), neutrophils, macrophages, basophils, and mast cells. Damage to the airway epithelium can be the inciting event, leading to the release of the cytokines IL-33, IL-25 and TSLP which active pulmonary innate lymphoid cells type 2 (ILC2). Pulmonary ILC2s in turn release IL-13 and IL-5 that then induce the full Type 2 immune response. IL-13, TIA and TL-5 production in the epithelium and mucosa of the lung leads to Goblet cell metaplasia and intraluminal mucin in the airways. Downstream activation of IL-6, IL-9, MCP-1 and eotaxin lead to airway infiltration by eosinophils, mast cells and basophils. These pathologic changes in the airways in turn lead to asthma exacerbations.

In additional studies described herein, it was observed that neutralization of IL-13 protects from pulmonary type 2 inflammation in a mouse model of COVID-19 and that, like IL-13, IL-4 is also elevated in COVID-19 patients with severe disease. IL-4 and IL-13 share a receptor, IL-4 receptor-alpha (IL-4Rx). For instance, IL-4 mediates its biological effects by binding to IL-4Ra. The IL-4/IL-4Ra complex subsequently recruits either yc to form the Type I IL-4 receptor or IL-13 receptor alpha 1 (IL-13Rα1) to form the Type II IL-4 receptor. Therefore IL-4 signals via both the Type I and II receptors. IL-13 in contrast signals via the Type II receptor composed of IL-4Ra and IL-13Rα1. The Type I receptor is expressed mostly in lymphocytes while the Type II receptor is predominantly expressed in non-hematopoietic cells. Myeloid cells (monocytes and granulocytes) have both Type I and Type II receptors (Junttila 2018).

While IL-13 is a key inducer of acute inflammation in the lung, IL-4 is also capable of inducing pulmonary inflammation. Studies in IL-13Rα1 knockout mice demonstrated that IL-13Rα1 signaling was involved in increased airway resistance, mucus, TGF-beta, and eotaxin(s) production, but was not required for Th2 and IgE responses to T cell-dependent antigens. Migration of eosinophils into the lung was independent of IL-13Rα1 signaling, highlighting complementary and redundant roles for IL-4 and IL-13 in pulmonary inflammation (Munitz et al 2008). In mice the intranasal administration of either IL-4 or IL-13 drove airway inflammation characterized by increases in alveolar macrophages, eosinophils and neutrophils and Goblet cell metaplasia. Eotaxin and IL-5 were induced by IL-4, and both IL-13 and IL-4 caused eosinophil infiltration into the lung (Le Floc'h et al 2020). That this is believed to be due to in part to the shared IL-4Rα1 receptor by which both IL-4 and IL-13 signal was demonstrated by protection from pulmonary inflammation by neutralization of IL-4Rα1 (Le Floc'h et al 2020).

According to some aspects the presently disclosed subject matter is based on the observation that prior treatment with dupilumab, which targets IL-4Ra, and thus neutralizes both IL-13 and IL-4, is associated with a reduced risk of severe disease in COVID-19 patients. In some embodiments, the presently disclosed subject matter is based, in part, on data showing that plasma levels of combinations of cytokines including the combination of IL-13 and IL-6 or the combination of IL-13, IL-6, IL-8 and MIP-13 are predictive of disease severity in COVID-19.

### III. Methods of Treating COVID-19

In some embodiments, the presently disclosed subject matter provides a method of treating COVID-19 in a subject in need thereof, e.g., a human subject suffering from and/or diagnosed with a SARS-CoV-2 infection. In some embodiments, the method comprises administering to the subject a composition that comprises a therapeutically effective amount of at least one agent that neutralizes one or more of interleukin-13 (IL-13), interleukin-6 (IL-6) and Janus kinase (JAK). The term “neutralizes” refers to an agent that binds to the named “neutralized” entity or a biological binding partner (e.g., receptor) thereof, thereby decreasing or inhibiting binding of the named neutralized entity and its binding partner, and/or that otherwise decreases or inhibits one or more biological activity of the named neutralized entity (e.g., IL-13). In some embodiments, the neutralization can result in a decrease in a biological activity by at least about 10%, about 20%, about 30%, about 40%, about 50%, about 60%, about 70%, about 80%, about 90%, or about 95% or more, as compared to the level of biological activity of the named entity in the absence of the neutralizing agent. The at least one agent can be, for example, a small molecule (e.g., a natural or synthetic compound with a molecular weight of less than about 900 Daltons (Da), less than about 800 Da, less than about 700 Da, or less than about 600 Da); a protein or peptide; an antibody, an antigen binding portion thereof or a biosynthetic antibody binding site that binds a particular target protein; or an antisense molecule (e.g., a short interfering RNA (siRNA)) that hybridizes in vivo to a nucleic acid encoding a protein or protein receptor (e.g., IL13Rα1 or IL4Ra) or mRNA or a fragment or regulatory element associated therewith.

In some embodiments, the composition comprises a therapeutically effective amount of at least one agent that neutralizes IL-13. In some embodiments, the at least one agent that neutralizes IL-13 also neutralizes IL-4, and its use can result in dual neutralization of IL-13 and IL-4. In some embodiments, the at least one agent results in dual neutralization of IL-13 and IL-4 by binding to IL4Rα1.

In some embodiments, the at least one agent that neutralizes IL-13 (or IL-13 and IL4) is an antibody or antibody fragment. In some embodiments, the antibody is a monoclonal antibody (mAb). Anti-IL-13 antibodies are known in the art and/or can be prepared using methods known in the art, for instance, using IL-13 or a fragment thereof in an antigen. Methods of preparing anti-IL-13 antibodies are described e.g., in U.S.

Patent Application Serial Nos. 2005/0065327, 2016/0272706; 2017/0340737; and 2019/0008966; the disclosure of each of which is incorporated herein by reference in its entireity. Sequences of human IL-13 (Uniprot No. P35225) include:
mhpllnplll alglmalllt tvialtclgg faspgpvpps talrelieel vnitqnqkap lcngsmvwsi nltagmycaa leslinvsgc saiektqrml sgfcphkvsa gqfsslhvrd tkievaqfvk dlllhlkklf regqfn (SEQ ID NO: 1, interleukin-13 isoform a precursor [Homo sapiens], NCBI Reference Sequence: NP_002179, 146 amino acids); and
mvwsinltag mycaalesli nvsgcsaiek tqrmlsgfcp hkvsagqfss lhvrdtkiev aqfvkdlllh lkklfregqf n (SEQ ID NO: 2, interleukin-13 isoform b [Homo sapiens], NCBI Reference Sequences: NP_001341920, NP_001341921, NP_001341922, 81 amino acids); and

In addition, antibody agents that neutralize IL-13 can include antibodies that bind to IL13Rα1 (Uniprot No. P78552) or IL4Ra (Uniprot No. P24394). Anti-ILRRα1 and anti-IL4Rα antibodies are known in the art. Commercially available anti-IL13Rα1 antibodies include, for example, catalog numbers AB246519, AB79277, and AB27337 (Abcam, Cambridge, United Kingdom). Additional antibodies can be prepared via methods known in the art by using IL13Rα1 or IL4Ra, or a fragment thereof, as an antigen. Sequences of human IL13Rα1 and IL4Rα are:

mewparlcgl walllcaggg gggggaapte tqppvtnlsv
svenlctviw twnppegass ncslwyfshf gdkqdkkiap
etrrsievpl nericlqvgs qcstnesekp silvekcisp
pegdpesavt elqciwhnls ymkcswlpgr ntspdtnytl
yywhrsleki hqcenifreg qyfgcsfdlt kvkdssfeqh
svqimvkdna gkikpsfniv pltsrvkpdp phiknlsfhn
ddlyvqwenp qnfisrclfy evevnnsqte thnvfyvqea
kcenpefern ventscfmvp gvlpdtlntv rirvktnklc
yeddklwsnw sqemsigkkr nstlyitmll ivpvivagai
ivlllylkrl kiiifppipd pgkifkemfg dqnddtlhwk
kydiyekqtk eetdsvvlie nlkkasq (SEQ ID NO: 3,
interleukin-13 receptor alpha 1 [homo sapiens],
NCBI Reference Sequence: NP_001551, 421 amino
acids);
and
mgwlcsgllf pvsclvllqv assgnmkvlq eptcvsdyms
istcewkmng ptncstelrl lyqlvfllse ahtcipenng
gagcvchllm ddvvsadnyt ldlwagqqll wkgsfkpseh
vkprapgnlt vhtnvsdtll ltwsnpyppd nylynhltya
vniwsendpa dfriynvtyl epslriaast lksgisyrar
vrawaqcynt twsewspstk whnsyrepfe qhlllgvsvs
civilavcll cyvsitkikk ewwdqipnpa rsrlvaiiiq
daqgsqwekr srgqepakcp hwkncltkll pcflehnmkr
dedphkaake mpfqgsgksa wcpveisktv lwpesisvvr
cvelfeapve ceeeeeveee kgsfcaspes srddfqegre
givarltesl fldllgeeng gfcqqdmges cllppsgsts
ahmpwdefps agpkeappwg keqplhleps ppasptqspd
nltctetplv iagnpayrsf snslsqspcp relgpdplla
rhleevepem pcvpqlsept tvpqpepetw eqilrrnvlq
hgaaaapvsa ptsgyqefvh aveqggtqas avvglgppge
agykafssll assavspekc gfgassgeeg ykpfqdlipg
cpgdpapvpv plftfgldre pprspqsshl pssspehlgl
epgekvedmp kpplpqeqat dplvdslgsg ivysaltchl
cghlkqchgq edggqtpvma spccgcccgd rssppttplr
apdpspggvp leaslcpasl apsgiseksk ssssfhpapg
naqsssqtpk ivnfvsvgpt ymrvs (SEQ ID NO: 4,
interleukin-4 receptor subunit alpha isoform
a precursor [homo sapiens], NCBI Reference
Sequence: NP_00409.1, 825 amino acids).

In some embodiments, the anti-IL-13 antibodies of use in the presently disclosed methods include, but are not limited to, dupilumab, lebrikizumab, and tralokinumab. Dupilumab (sold under the tradename DUPIXENT® Sanofi Biotechnology, Paris, France), developed by Sanofi Genzyme (Cambridge, Mass., United States of America) and Regeneron Pharmaceuticals (Tarrytown, N.Y., United States of America), has been approved in by the FDA for use in treating moderate to severe atopic dermatitis and asthma. Dupilumab is an anti-human IL4Rα antibody that can block the IL4/IL4Rα signaling pathway. Lebrikizumab is a mAb that targets IL-13 and prevents IL-13Rα1/IL-4Rα heterodimer receptor signaling complex formation and is in clinical trials for use in treating atopic dermatitis (Guttman-Yassky et al., 2020). Tralokinumab was initially developed by AstraZeneca (Cambridge, United Kingdom) and later by Leo Pharma (Ballerup, Denmark. Tralokinumab is a mAb that targets IL-13 and has been used to treat atopic dermatitis (Wollenberg et al., 2019). In some embodiments, the agent that neutralizes IL-13 is dupilumab or another agent that targets IL4Rα1.

The anti-IL13 antibodies can be administered in the present methods using similar routes and/or dosages to those used with the antibodies for treating other diseases. For instance, in some embodiments, dupilumab can be administered according to the presently disclosed method via routes and using dosages similar to those used when dupilumab is used to treat atopic dermatitis, e.g., via sub-cutaneous injection using a loading dose of 600 milligrams (as two 300 mg doses), with additional 300 mg doses every two weeks, as needed.

In view of the observation of elevated IL-6 in COVID-19 patients and the observation that blocking IL-13 can block IL-6 expression, agents that neutralize IL-6 can also be used. Thus, in some embodiments, the composition comprises a therapeutically effective amount of at least one agent that neutralizes IL-6 (Uniprot No. P05231). In some embodiments, the agent that neutralizes IL-6 is an antibody or a fragment thereof. In some embodiments, the agent is a mAb. Anti-IL-6 antibodies are known in the art, including, but not limited to, tocilizumab, sarilumab, and siltuximab. More particularly, tocilizumab (sold under the brand name ACTEMRA@) and sarilumab (sold under the brand name KEVZARA@) both target the IL-6 receptor, while siltuximab (sold under the brand name SYLVANT@) binds to IL-6 itself. In some embodiments, the agent that neutralizes IL-6 is used in combination with an agent that neutralizes IL-13 and the composition comprises at least one agent that neutralizes IL-13 and at least one agent that neutralizes IL-6.

In some embodiments, an antisense molecule can be used to neutralize IL-13 or IL-6. As used herein, an “antisense” molecule includes an RNA molecule which, by binding to a complementary sequence in RNA, inhibits the function and/or completion of synthesis of the latter molecule. In some embodiments, the antisense molecule can target a nucleic acid that encodes one of SEQ ID NOs: 1-4 or a fragment thereof. In some embodiments, the antisense molecule can target a nucleic acid that encodes IL-6, such as a sequence that encodes one of the amino acid sequences corresponding to NCBI Reference Sequence NP_000591.1 (SEQ ID NO: 5), NP_001305024.1 (SEQ ID NO: 6) and NP_001358025.1 (SEQ ID NO: 7), or a fragment thereof. In some embodiments the antisense molecule can target an IL-13, IL13Rα1, or IL4Rα mRNA sequence (one of SEQ ID NOs: 8-16) in the table below, or a fragment thereof. In some embodiments, the antisense molecule can target an IL-6 mRNA sequence (one of SEQ ID NOs 17-19) in the table below, or a fragment thereof.

| | | SEQ |
|---|---|---|
| | | ID |
| Sequence | NCBI Ref. No. | NO. |
| IL-13 mRNA, homo sapiens, transcript variant 1 | NM_002188.3 | 8 |
| IL-13 mRNA, homo sapiens, transcript variant 2 | NM_001354991.2 | 9 |
| IL-13 mRNA, homo sapiens, transcript variant 3 | NM_001354992.2 | 10 |
| IL-13 mRNA, homo sapiens, transcript variant 4 | NM_001354993.2 | 11 |
| IL13 receptor subunit alpha 1, homo sapiens | NM_001560.3 | 12 |
| IL4 receptor, homo sapiens | NM_000418.4 | 13 |
| IL4 receptor, transcript variant 3, homo sapiens | NM_001257406.2 | 14 |
| IL4 receptor, transcript variant 4, homo sapiens | NM_001257407.2 | 15 |
| IL4 receptor, transcript variant 5, homo sapiens | NM_001257997.2 | 16 |
| IL-6 mRNA, transcript variant 1, homo sapiens | NM_000600.5 | 17 |
| IL-6 mRNA, transcript variant 2, homo sapiens | NM_001318095.2 | 18 |
| IL-6 mRNA, transcript variant 3, homo sapiens | NM_001371096.1 | 19 |

Given the role of IL-13 in COVID-19 described herein, Janus kinase inhibitors can also be used in the presently disclosed methods. Janus kinase refers to a family of tyrosine kinases that are involved in cytokine signaling. A variety of JAK inhibitors are known in the art and/or are available commercially (e.g., from companies such as Millipore Sigma (Burlington, Mass., United States of America), MedChemExpress (Monmouth Junction, N.J., United States of America), and the like). In some embodiments, the JAK inhibitor is a small molecule. Various JAK inhibitors have been tested clinical trials and/or are under development for treating dermatological and/or autoimmune disorders, such as psoriasis, rheumatoid arthritis (RA), lupus, and atopic dermatitis. These inhibitors include, but are not limited to, baricitinib, tofacitinib, ruxolitinib, GSK2586184, Abrocitinib (PF-0496582), CYT387, AZD1480, GLPG-0634, TG101349, AC-439, CEP-33779, lestaurtinib, pacritinib, BMS-911543, VX509, and R-348 (Furumoto and Gradina (2013); Shreberk-Hassidim (2017)). Baricitinib, for example, has been approved for use in RA by the FDA (Mogul et al., (2019). Thus, in some embodiments, the composition of the presently disclosed method includes a therapeutically effective amount of at least one agent that neutralizes or inhibits JAK. In some embodiments, the agent that neutralizes or inhibits JAK is used in combination with an agent that neutralizes IL-13 and the composition comprises at least one agent that neutralizes IL-13 and at least one agent that neutralizes JAK.

In some embodiments, the JAK inhibitor is an agent that targets Janus kinase 1 (JAK1), e.g., by being a non-selective JAK inhibitor or by being an inhibitor that selectively targets JAK1. JAK1, for example, is downstream of IL-13 in the IL-13 signaling pathway and can interact with the IL4 receptor family. In some embodiments, the JAK inhibitor is selected from the group including, but not limited to, baricitinib, tofacitinib, ruxolitinib, GSK2586184, GLPG-0634, CYT387, AZD1480, abrocitinib (PF-0496582), peficitinib, filgotinib, itacitinib, delgocitinib, momelotinib, CAS-457081-03-7. In some embodiments, the JAK inhibitor is selected from baricitinib and tofacitinib.

In some embodiments, treating according to the presently disclosed method provides a reduction in disease severity and/or improved disease outcome, e.g., compared to the expected disease severity or outcome in the absence of the treating. Reducing disease severity and/or improving disease outcome can provide one or more of the following: reduced pulmonary inflammation, shorter disease duration, reduced weight loss, reduced risk of ventilation dependence, reduced risk of hospitalization, shorter hospitalization, and/or reduced risk of death. In some embodiments, treating reduces pulmonary inflammation. In some embodiments, treating reduces the risk of one or more of ventilation dependence, hospitalization, and death.

In some embodiments, the subject in need of treatment is a mammal. In some embodiments, the subject is a human. In some embodiments, the subject is a subject who has a SARS-CoV-2 infection, is suspected of having a SARS-CoV-2 infection (e.g., by exhibiting one or more symptoms of a SARS-CoV-2 infection), or is a subject who has one or more increased risk factors for a SARS-CoV-2 infection. Increased risk factors for getting a SARS-CoV-2 infection include, but are not limited to, contact with another who has tested positive for SARS-CoV-2 infection or who has exhibited symptoms of SARS-CoV-2 infection, e.g., fever, chills, cough, difficulty breathing, headache, fatigue, sore throat, muscle or body ache, loss of smell or taste, nausea, and diarrhea; living in communal housing (e.g., a nursing home); and travel to areas with high rates of SARS-CoV-2 infectivity. In some embodiments, the subject is a subject who has increased risk for severe COVID-19 (e.g., risk for disease requiring hospitalization, ventilation or oxygen support, and/or that results in death). Subjects with increased risk of severe disease include human subjects over the age of 45 or over the age of 65. Underlying medical conditions or comorbidities that can result in higher risk of severe COVID-19 include, but are not limited to, cancer, chronic kidney disease, chronic lung disease (e.g., chronic obstructive pulmonary disease (COPD), asthma, cystic fibrosis, pulmonary hypertension, and interstitial lung disease), dementia, diabetes, Down's syndrome, heart disease (e.g., heart failure, coronary artery disease, hypertension, etc.), HIV, a compromised or weakened immune system, liver disease (e.g., cirrhosis), being overweight (i.e., having a body mass index (BMI)>25 kg/m²) or obese (i.e., having a BMI≥30 kg/m²), pregnancy, sickle cell disease or thalassemia, a history of smoking, stroke, and having a history of substance abuse.

The compositions (e.g., antibodies) of the presently disclosed subject matter are administered to the subject in a therapeutically effective amount (i.e., an amount that has a desired therapeutic effect). Typically, antibodies can be administered parenterally. However, the compositions can be administered via any suitable route. The dose and dosage regimen can depend upon the degree of disease severity, the characteristics of the particular therapeutic agent used, e.g., its therapeutic index, the patient, and the patient's history. In some embodiments, an antibody agent of the presently disclosed methods is administered continuously over a period of 1-2 weeks.

In some embodiments, a therapeutic dose of an antibody of the presently disclosed subject matter, including, but not limited to, monoclonal antibodies, chimeric antibodies, humanized antibodies, various kinds of fragments, and biologically active homologs and fragments thereof, is from about 0.1 mg/dose to about 5,000 mg/dose or from about 0.2 mg/dose to about 1,000 mg/dose. Doses can also be administered based on body weight, for example at a dosage ranging from about 0.01 mg/kg to about 1,000 mg/kg body weight or from about 0.1 to about 500 mg/kg.

The total amount to be administered during a day can be divided into lower doses and administered at multiple times/day. In some embodiments, the method is useful for low dose treatment. In some embodiments, the method is useful for short-term treatment. For example, if 20 mg/kg/day is the prescribed amount for the day, that amount can be divided into more than one dose for administration during the day, such as doses of 10 mg/kg administered twice. In some embodiments, treatment can be as short as 1 day. In some embodiments, even doses as low as 0.01, 0.1, 0.5, 1.0, 1.5, 2.0, 2.5, 3.0, 3.5, 4.0, 4.5, and 5.0 mg/kg/day can be administered as partial doses multiple times in a day when it is determined that the entire daily dose does not need to be administered in one bolus or that it would be better to administer the daily dose in several increments.

One of ordinary skill in the art can determine the best route of administration of a pharmaceutical composition of the presently disclosed subject matter. For example, administration can be direct, enteral, or parenteral. Enteral includes, for example, oral and rectal administration. Parenteral includes, for example, intravenous administration. One of ordinary skill in the can determine the method and site of administration. For example, enteral, parental, direct, intravenous, or subcutaneous injection a composition comprising a protein agent (or biologically active fragments or homologs thereof), such as an antibody or cytokine, can be an effective treatment.

A compound or composition of the presently disclosed subject matter can be administered once or more than once. It can be administered once a day or at least twice a day. In one aspect, a compound is administered every other day within a chosen term of treatment. In one embodiment, at least two compounds of the presently disclosed subject matter are used. One of ordinary skill in the art can determine how often to administer a compound of the presently disclosed subject matter, the duration of treatment, and the dosage to be used.

When two or more compounds are to be administered, they can be administered in the same pharmaceutical composition or in separate pharmaceutical compositions. When administered in separate pharmaceutical compositions, they can be administered simultaneously or one can be administered first. The amount of time between administration of the different compounds can vary and can be determined by one of ordinary skill in the art. For example, the two compounds could be administered up to 10 minutes apart, up to 30 minutes apart, up to 1 hour apart, etc. In one aspect, one or more of the compounds can be administered more than once. In one aspect, a compound is administered at least twice. In another aspect, a compound is administered at least five times. In one aspect, the method is useful for low dose treatment. In one aspect, the method is useful for short-term treatment.

In some embodiments, duration of treatment is from about 1-10, 1-9, 1-8, 1-7, 1-6, 1-5, 1-4, 1-3, or 1-2 days. In some embodiments, duration of treatment is from about 2-10, 2-9, 2-8, 2-7, 2-6, 2-5, 2-4, or 2-3 days. In some embodiments, duration of treatment is from about 3-10, 3-9, 3-8, 3-7, 3-6, 3-5, or 3-4 days. In some embodiments, duration of treatment is from about 4-10, 4-9, 4-8, 4-7, 4-6, or 4-5 days. In some embodiments, duration of treatment is from about 5-10, 5-9, 5-8, 5-7, or 5-6 days. In some embodiments, duration of treatment is from about 6-10, 6-9, 6-8, or 6-7 days. In some embodiments, duration of treatment is from about 7-10, 7-9, or 7-8 days. In some embodiments, duration of treatment is from about 8-10 or 8-9. In some embodiments, treatment is for about 1, 2, 3, 4, 5, 6, 7, 8, 9, or 10 days.

In some embodiments, a subject is treated daily during the treatment regimen when the duration is longer than one day. In another aspect, the subject is treated every other day.

### IV. Methods of Predicting Risk in COVID-19

In some embodiments, the presently disclosed subject matter provides a method of predicting increased risk of severe disease in a subject with COVID-19 (i.e., a subject who has tested positive for SARS-CoV-2 infection). The method can be used, for example, to better tailor treatment to an individual patient. In some embodiments, “increased risk” as used herein refers to the subject having a higher likelihood of having a severe disease outcome than the average risk for a similar subject (i.e., a subject of about the same age and/or physical condition) to have severe disease. In some embodiments, the method predicts increased risk for a subject to require mechanical ventilation or oxygen support during treatment. For example, in some embodiments, the presently disclosed subject matter provides a method of predicting increased risk for mechanical ventilation in a subject having COVID-19, where the method comprises: (a) measuring a plasma concentration of one or more cell signaling protein selected from the group including IL-13, IL-7, and FGF-basic (bFGF) in the subject; (b) comparing the plasma concentration measured in the subject with a reference concentration (or “reference value”) of the one or more cell signaling protein (e.g., the average concentration of the cell signaling protein in age-matched healthy subjects); and (c) predicting if the subject has increased risk for mechanical ventilation. For instance, if the plasma concentration in the subject (i.e., the plasma concentration measured in step (a)) is higher than the reference concentration, the subject is predicted as having increased risk of mechanical ventilation. If the plasma concentration measured in the subject (i.e., the plasma concentration measured in step (a)) is not higher than the higher than the reference concentration, the subject is predicted as not having increased risk for mechanical ventilation.

In some embodiments, the prediction is used to develop or modify a medical treatment plan for the subject. For example, if the subject is predicted to have increased risk of mechanical ventilation, the method can further comprise administering to the subject a composition that comprises a therapeutically effective amount of at least one agent that neutralizes one or more of IL-13, IL-6, and JAK. If the subject is not predicted as having an increased risk of mechanical ventilation, the subject will not be treated with the composition. The compositions comprising agents that neutralize IL-13, IL-6 and/or JAK can be the same as those described hereinabove. For example, in some embodiments, the composition comprises a therapeutically effective amount of an antibody that neutralizes IL-13 (e.g., by binding IL-13, IL-13Rα, or IL4Rα). In some embodiments, the composition comprises a monoclonal antibody selected from the group including dupilumab, lebrikizumab, and tralokinumab. In some embodiments, the composition comprises dupilumab. In some embodiments, the composition comprises a therapeutically effective amount of at least one agent that neutralizes IL-6, e.g., a monoclonal antibody such as, but not limited to, tocilizumab and sarilumab. In some embodiments, the composition comprises a therapeutically effective amount of at least one agent that neutralizes JAK (e.g., JAK1), e.g., a small molecule JAK inhibitor such as baricitinib or tofacitinib.

In some embodiments, step (a) comprises measuring a plasma concentration of IL-13 in the subject, either alone or in combination with measuring the concentration of one or more additional cell signaling protein (e.g., another cytokine). In some embodiments, step (a) further comprises measuring a plasma concentration of IL-6. In some embodiments, step (a) further comprises measuring a plasma concentration of IL-8 and MIP-13. Accordingly, in some embodiments, step (a) comprises measuring a plasma concentration of IL-13 and a plasma concentration of IL-6 in the subject. In some embodiments, step (a) comprises measuring a plasma concentration of each of IL-13, IL-6, IL-8, and MIP-1β.

Plasma concentrations of cell signaling proteins can be measure by methods known in the art. For example, plasma concentrations of the proteins can be measured using antibody-based detection methods, such as enzyme-linked immunosorbent assay (ELISA). Plasma concentrations of the cell signaling proteins measured using commercially available multi-cytokine assay kits, such as those available from R & D Systems (Minneapolis, Minn., United States of America) and Millipore Sigma, (Burlington, Mass., United States of America).

In some embodiments, the “reference concentration” or “reference value” can refer to a threshold value or a cut-off value. The setting of a single “reference value” thus allows discrimination between a predicting increased risk of mechanical ventilation and a normal risk of mechanical ventilation in a COVID-19 patient. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. The threshold value can be determined in order to obtain the optimal sensitivity and specificity according to the function of the test and the benefit/risk balance (clinical consequences of false positive and false negative). In some embodiments, the optimal sensitivity and specificity (and so the threshold value) can be determined using a Receiver Operating Characteristic (ROC) curve based on experimental data. Particularly, the person skilled in the art can compare the concentration (obtained according to the method of the presently disclosed subject matter) with a defined threshold value. In some embodiments, the threshold value can be derived from the plasma concentration determined in plasma samples derived from one or more patients having COVID-19. Furthermore, retrospective measurement of the plasma concentration in properly banked historical patient samples can be used in establishing these threshold values.

Predetermined reference values used for comparison can comprise “cut-off” or “threshold” values that can be determined as described herein. Each reference (“cut-off”) value can be predetermined by carrying out a method comprising the steps of: (i) providing a collection of plasma samples from COVID-19 patients; (ii) determining the plasma concentration of one or more cell signaling protein including at least one of IL-13, IL-7, and bFGF for each sample contained in the collection provided at step (i); (iii) ranking the samples according to concentration of said cell signaling protein; (iv) classifying said samples in pairs of subsets of increasing, respectively decreasing, number of members ranked according to their concentration; (v) providing, for each sample provided at step a), information relating to the actual clinical outcome for the COVID-19 patient (e.g., number of days of mechanical ventilation or hospitalization); (vi) for each pair of subsets of samples, obtaining a Kaplan Meier percentage of survival curve; (vii) for each pair of subsets of samples calculating the statistical significance (p value) between both subsets; and (viii) selecting as reference value for the plasma concentration, the plasma concentration for which the p value is the smallest.

### V. Antibodies, Peptides and Proteins

Antibodies directed against proteins, polypeptides, or peptide fragments thereof of the presently disclosed subject matter can be generated using methods that are well known in the art. For instance, U.S. Pat. No. 5,436,157, which is incorporated by reference herein in its entirety, discloses methods of raising antibodies to peptides. For the production of antibodies, various host animals, including but not limited to rabbits, mice, and rats, can be immunized by injection with a polypeptide or peptide fragment thereof. To increase the immunological response, various adjuvants can be used depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum.

In some embodiments, antibodies or antisera, directed against IL-13, IL13Rα1, or IL-4Rα or a homolog or fragment thereof, are useful for blocking the activity of IL-13 and/or IL-4.

Fragments of IL-13, IL-13Rα1, or IL-4Rα can be generated and antibodies prepared against the fragments. For the preparation of monoclonal antibodies, any technique which provides for the production of antibody molecules by continuous cell lines in culture can be utilized. For example, the hybridoma technique originally developed by Kohler and Milstein (1975, Nature 256:495-497), the trioma technique, the human B-cell hybridoma technique (Kozbor et al., 1983, Immunology Today 4:72), and the EBV-hybridoma technique (Cole et al., 1985, in Monoclonal Antibodies and Cancer Therapy, Alan R. Liss, Inc., pp. 77-96) can be employed to produce human monoclonal antibodies. In some embodiments, monoclonal antibodies are produced in germ-free animals.

In some embodiments, human antibodies can be used and obtained by utilizing human hybridomas (Cote et al., 1983, Proc. Natl. Acad. Sci. U.S.A. 80:2026-2030) or by transforming human B cells with EBV virus in vitro (Cole et al., 1985, in Monoclonal Antibodies and Cancer Therapy, Alan R. Liss, Inc., pp. 77-96). Furthermore, techniques developed for the production of “chimeric antibodies” (Morrison et al., 1984, Proc. Natl. Acad. Sci. U.S.A. 81:6851-6855; Neuberger et al., 1984, Nature 312:604-608; Takeda et al., 1985, Nature 314:452-454) by splicing the genes from a mouse antibody molecule specific for epitopes of IL-13, IL-13Rα1 or IL-4Rα polypeptides together with genes from a human antibody molecule of appropriate biological activity can be employed; such antibodies are within the scope of the presently disclosed subject matter. Once specific monoclonal antibodies have been developed, the preparation of mutants and variants thereof by conventional techniques is also available.

In some embodiments, techniques described for the production of single-chain antibodies (U.S. Pat. No. 4,946,778, incorporated by reference herein in its entirety) are adapted to produce protein-specific single-chain antibodies. In another embodiment, the techniques described for the construction of Fab expression libraries (Huse et al., 1989, Science 246:1275-1281) are utilized to allow rapid and easy identification of monoclonal Fab fragments possessing the desired specificity for specific antigens, proteins, derivatives, or analogs of the presently disclosed subject matter.

Antibody fragments which contain the idiotype of the antibody molecule can be generated by known techniques. For example, such fragments include but are not limited to: the F(ab′)₂ fragment which can be produced by pepsin digestion of the antibody molecule; the Fab′ fragments which can be generated by reducing the disulfide bridges of the F(ab′)₂ fragment; the Fab fragments which can be generated by treating the antibody molecule with papain and a reducing agent; and Fv fragments.

The generation of polyclonal antibodies is accomplished by inoculating the desired animal with the antigen and isolating antibodies which specifically bind the antigen therefrom.

Monoclonal antibodies directed against full length or peptide fragments of a protein or peptide can be prepared using any well-known monoclonal antibody preparation procedures, such as those described, for example, in Harlow et al. (1988, In: Antibodies, A Laboratory Manual, Cold Spring Harbor, N.Y.) and in Tuszynski et al. (1988, Blood, 72:109-115). Quantities of the desired peptide can also be synthesized using chemical synthesis technology. Alternatively, DNA encoding the desired peptide can be cloned and expressed from an appropriate promoter sequence in cells suitable for the generation of large quantities of peptide. Monoclonal antibodies directed against the peptide are generated from mice immunized with the peptide using standard procedures as referenced herein.

A nucleic acid encoding the monoclonal antibody obtained using the procedures described herein can be cloned and sequenced using technology which is available in the art, and is described, for example, in Wright et al. (1992, Critical Rev. in Immunol. 12(3,4):125-168) and the references cited therein. Further, the antibody of the presently disclosed subject matter can be “humanized” using the technology described in Wright et al., (supra) and in the references cited therein, and in Gu et al. (1997, Thrombosis and Hematocyst 77(4):755-759).

To generate a phage antibody library, a cDNA library is first obtained from mRNA which is isolated from cells, e.g., the hybridoma, which express the desired protein to be expressed on the phage surface, e.g., the desired antibody. cDNA copies of the mRNA are produced using reverse transcriptase. cDNA which specifies immunoglobulin fragments are obtained by PCR and the resulting DNA is cloned into a suitable bacteriophage vector to generate a bacteriophage DNA library comprising DNA specifying immunoglobulin genes. The procedures for making a bacteriophage library comprising heterologous DNA are well known in the art and are described, for example, in Sambrook et al. (1989, Molecular Cloning: A Laboratory Manual, Cold Spring Harbor, N.Y.).

Bacteriophage that encode the desired antibody can be engineered such that the protein is displayed on the surface thereof in such a manner that it is available for binding to its corresponding binding protein, e.g., the antigen against which the antibody is directed. Thus, when bacteriophage which express a specific antibody are incubated in the presence of a cell which expresses the corresponding antigen, the bacteriophage will bind to the cell. Bacteriophage which do not express the antibody will not bind to the cell. Such panning techniques are well known in the art.

Processes such as those described above have been developed for the production of human antibodies using M13 bacteriophage display (Burton et al., 1994, Adv. Immunol. 57:191-280). Essentially, a cDNA library is generated from mRNA obtained from a population of antibody-producing cells. The mRNA encodes rearranged immunoglobulin genes and thus, the cDNA encodes the same. Amplified cDNA is cloned into M13 expression vectors creating a library of phage which express human Fab fragments on their surface. Phage which display the antibody of interest are selected by antigen binding and are propagated in bacteria to produce soluble human Fab immunoglobulin. Thus, in contrast to conventional monoclonal antibody synthesis, this procedure immortalizes DNA encoding human immunoglobulin rather than cells which express human immunoglobulin.

The procedures presented above describe the generation of phage which encode the Fab portion of an antibody molecule. However, the presently disclosed subject matter should not be construed to be limited solely to the generation of phage encoding Fab antibodies. Rather, phage which encode single chain antibodies (scFv/phage antibody libraries) are also included in the presently disclosed subject matter. Fab molecules comprise the entire Ig light chain, that is, they comprise both the variable and constant region of the light chain but include only the variable region and first constant region domain (CH1) of the heavy chain. Single chain antibody molecules comprise a single chain of protein comprising the Ig Fv fragment. An Ig Fv fragment includes only the variable regions of the heavy and light chains of the antibody, having no constant region contained therein. Phage libraries comprising scFv DNA can be generated following the procedures described in Marks et al., 1991, J. Mol. Biol. 222:581-597. Panning of phage so generated for the isolation of a desired antibody is conducted in a manner similar to that described for phage libraries comprising Fab DNA.

The presently disclosed subject matter should also be construed to include synthetic phage display libraries in which the heavy and light chain variable regions can be synthesized such that they include nearly all possible specificities (Barbas, 1995, Nature Medicine 1:837-839; de Kruif et al. 1995, J. Mol. Biol. 248:97-105).

In the production of antibodies, screening for the desired antibody can be accomplished by techniques known in the art, e.g., ELISA (enzyme-linked immunosorbent assay). Antibodies generated in accordance with the presently disclosed subject matter can include, but are not limited to, polyclonal, monoclonal, chimeric (i.e., “humanized”), and single chain (recombinant) antibodies, Fab fragments, and fragments produced by a Fab expression library.

The peptides of the presently disclosed subject matter can be readily prepared by standard, well-established techniques, such as solid-phase peptide synthesis (SPPS) as described by Stewart et al. in Solid Phase Peptide Synthesis, 2nd Edition, 1984, Pierce Chemical Company, Rockford, Ill.; and as described by Bodanszky and Bodanszky in The Practice of Peptide Synthesis, 1984, Springer-Verlag, New York. At the outset, a suitably protected amino acid residue is attached through its carboxyl group to a derivatized, insoluble polymeric support, such as cross-linked polystyrene or polyamide resin. “Suitably protected” refers to the presence of protecting groups on both the α-amino group of the amino acid, and on any side chain functional groups. Side chain protecting groups are generally stable to the solvents, reagents and reaction conditions used throughout the synthesis, and are removable under conditions that will not affect the final peptide product. Stepwise synthesis of the oligopeptide is carried out by the removal of the N-protecting group from the initial amino acid, and couple thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group such as formation into a carbodiimide, a symmetric acid anhydride or an “active ester” group such as hydroxybenzotriazole or pentafluorophenly esters.

Examples of solid phase peptide synthesis methods include the BOC method that utilized tert-butyloxcarbonyl as the α-amino protecting group, and the FMOC method which utilizes 9-fluorenylmethyloxcarbonyl to protect the α-amino of the amino acid residues, both methods of which are well-known by those of skill in the art.

To ensure that the proteins or peptides obtained from either chemical or biological synthetic techniques is the desired peptide, analysis of the peptide composition should be conducted. Such amino acid composition analysis can be conducted using high resolution mass spectrometry to determine the molecular weight of the peptide. Alternatively, or additionally, the amino acid content of the peptide can be confirmed by hydrolyzing the peptide in aqueous acid, and separating, identifying and quantifying the components of the mixture using HPLC, or an amino acid analyzer. Protein sequenators, which sequentially degrade the peptide and identify the amino acids in order, can also be used to determine definitely the sequence of the peptide.

Prior to its use, the peptide can be purified to remove contaminants. In this regard, it will be appreciated that the peptide will be purified to meet the standards set out by the appropriate regulatory agencies. Any one of a number of a conventional purification procedures can be used to attain the required level of purity including, for example, reversed-phase high-pressure liquid chromatography (HPLC) using an alkylated silica column such as C₄-, C₈- or C₁₈-silica. A gradient mobile phase of increasing organic content is generally used to achieve purification, for example, acetonitrile in an aqueous buffer, usually containing a small amount of trifluoroacetic acid. Ion-exchange chromatography can be also used to separate peptides based on their charge.

Substantially pure peptide obtained as described herein can be purified by following known procedures for protein purification, wherein an immunological, enzymatic or other assay is used to monitor purification at each stage in the procedure. Protein purification methods are well known in the art, and are described, for example in Deutscher et al. (ed., 1990, Guide to Protein Purification, Harcourt Brace Jovanovich, San Diego).

The presently disclosed subject matter encompasses methods of screening compounds to identify those compounds that act as agonists (stimulate) or antagonists (inhibit) of the protein interactions and pathways described herein. Screening assays for antagonist compound candidates are designed to identify compounds that bind or complex with the peptides described herein, or otherwise interfere with the interaction of the peptides with other cellular proteins. Such screening assays will include assays amenable to high-throughput screening of chemical libraries, making them particularly suitable for identifying small molecule drug candidates.

### EXAMPLES

The following Examples has been included to provide guidance to one of ordinary skill in the art for practicing representative embodiments of the presently disclosed subject matter. In light of the present disclosure and the general level of skill in the art, those of skill can appreciate that the following Example is intended to be exemplary only and that numerous changes, modifications, and alterations can be employed without departing from the scope of the presently disclosed subject matter.

### Example 1

### IL-13, IL-7 and BFGF Association with Increased Risk for Ventilation in COVID-19 Patients

### Collection of Clinical Samples and Patient Information:

Patients tested for COVID-19 were found using the University of Virginia Medical Center's electronic database. Any remnant whole blood tubes were pulled by the clinical laboratory. Samples were collected within 48 hours of the time of diagnosis or hospitalization. Blood was centrifuged at 1300×g for 10 minutes, then plasma was aliquoted and stored at −80° C. until testing. Demographics (age, gender, race), comorbidities, hospitalization status, lab results, and other clinical information was pulled from the electronic health records database. The collection of biological specimens and de-identified patient information was approved by the Institutional Review Board (IRB-HSR #22231 and 200110).

### Measurement of Cytokines in Plasma

Cytokine concentrations in plasma were measured using a Pro Human Cytokine 27-plex Assay (R & D Systems, Minneapolis, Minn., United States of America) on a suspension array system sold under the tradename BIO-PLEX™ 200 suspension array system (Bio-Rad Laboratories, Hercules, Calif., Untied States of America). Cytokines detected were IL-1b, IL-1ra, IL-4, IL-5, IL-6, IL-7, IL-8, IL-9, IL-10, IL-12(p70), IL-13, IL-17, Eotaxin, FGF basic, G-CSF, IFN-g, IP-10, MCP-1(MCAF), MIP-1a, PDGF-bb, MIP-1b, RANTES and TNF-a. IL-2, IL-15 and GM-CSF were below the limits of detection.

### Statistical Analysis

The COVID-19 positive patients were separated by ventilator status and cytokine levels were compared using a Mann-Whitney U test. A Mann-Whitney U test was also performed to compare biomarkers between COVID-19 positive patients with elevated blood glucose (≥126 mg/dl) and patients with normal glucose (<125 mg/dl). Patient cytokine levels were separated into quartiles and log-rank tests were used to generate Kaplan-Meier curves. Odds ratios were calculated using general logistic regression, and confidence intervals that did not overlap 1 were considered statistically significant. For hierarchical clustering, the pheatmap function in the pheatmap library was used (R-project.org). Cytokines were scaled by row (patients), and the clustering was calculated using the complete linkage method. Statistical analyses were performed using GraphPad Prism and R.

### Results

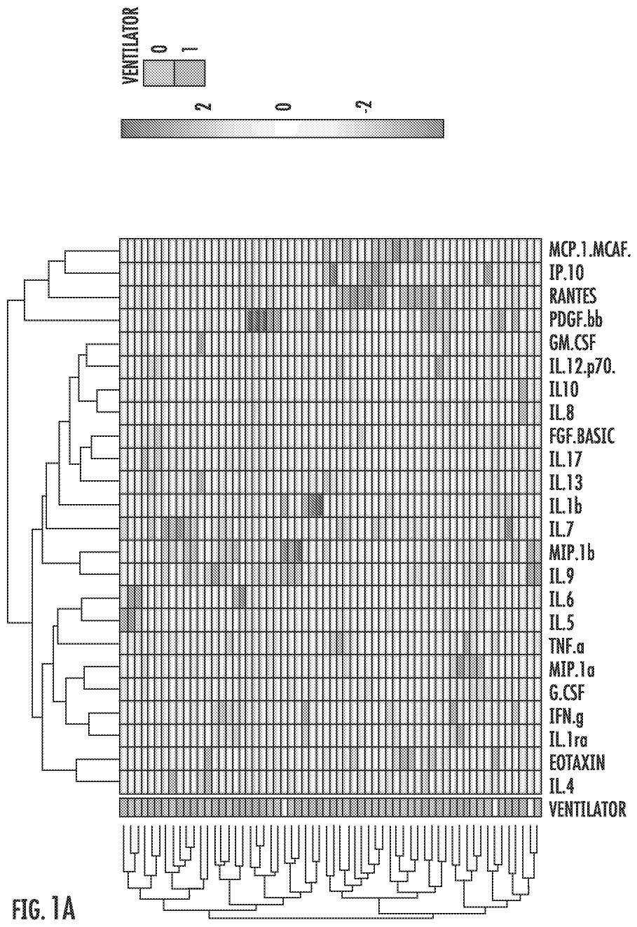

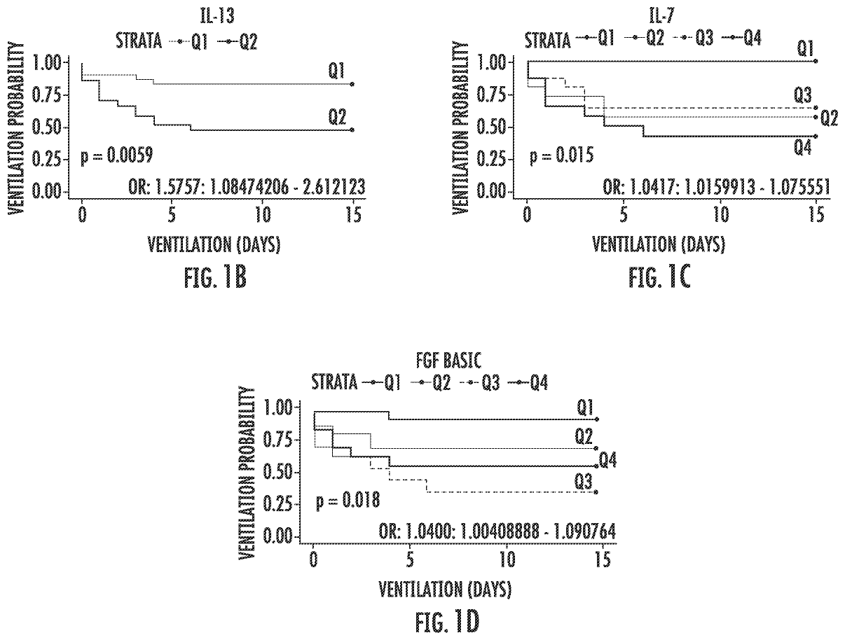

To understand the relationship between the different measured cytokines in the cohort, hierarchical clustering was used to generate a heatmap for patients and cytokines. See FIG. 1A. Cytokines that were clustered in close proximity with one another had similar expression dynamics within patients, compared to cytokines more distant. IL-6, a cytokine of interest for therapeutic intervention in COVID-19 patients, clustered most closely with IL-5, which is a type 2 cytokine that among other properties induces eosinophils (Sanderson, 1992). In addition, clustering between IL-8 and IL-10 was observed. Without being bound to any one theory, this is believed due to their induction during CRS. Interestingly, these two cytokines did not cluster closely with IL-6. Of note there was little visible separation between patients who were ventilated compared to those who were not through clustering analysis, and no significant observable pattern in the heatmap. This highlighted the heterogeneity in responses between patients.

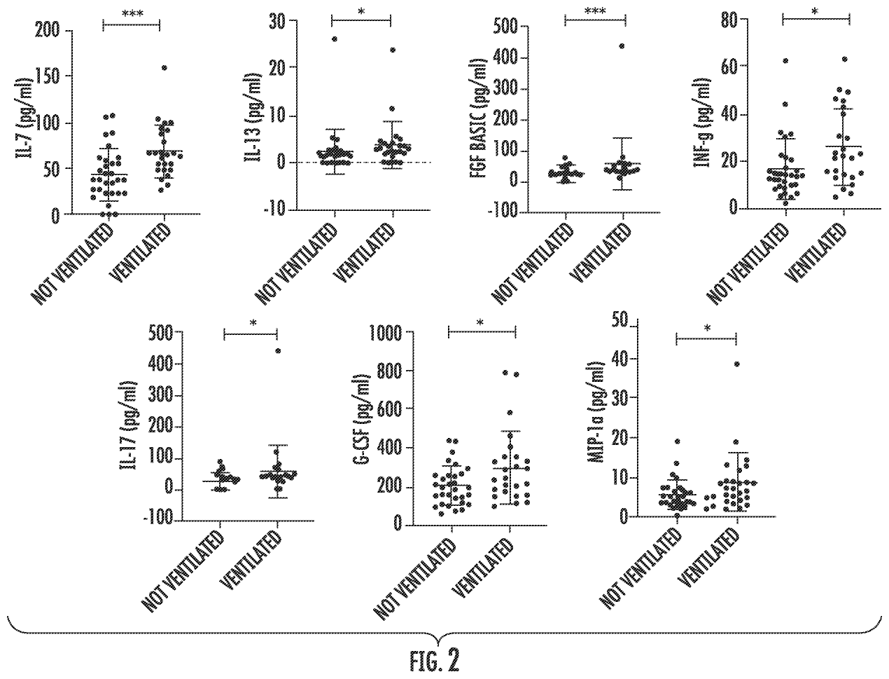

Seven cytokines were found to be elevated in COVID-19 patients that ultimately required ventilation. See FIG. 2. These included FGF basic (p=0.0006), IL-7 (p=0.0006), IFN-g (p=0.0102), MIP-1α(p=0.0195), IL-13 (p=0.0159), G-CSF (p=0.0368), and IL-17 (p=0.0448). IL-1ra (p=0.0554) and IL-1b (p=0.0707) were also trending towards being significantly higher in the ventilated patients. For the seven cytokines with elevated expression in ventilated patients, a logistic regression was performed to calculate the odds ratio for ventilation status in the patients, while controlling for gender, age, and presence of comorbidities. For cytokines with significant odds ratios, the expression levels were then divided into halves or quartiles and Kaplan-Meier curves for time-to-ventilation after admission to the hospital were generated. From this, three cytokines were found to have significant odds ratios and Kaplan-Meier curves: IL-13, IL-7, and FGF-basic. See FIGS. 1B-1D. Patients in the top half percentile of IL-13 were significantly more likely to require ventilation (p=0.005). Additionally, IL-13 levels were predictive of ventilation, with an odds ratio of 1.58. IL-7, which is related to induction and maintenance of lymphocytes (Fry and Mackall, 2005), was also predictive of ventilation (OR: 1.04); and patients in the top three percentiles for IL-7 expression were significantly more likely to require ventilation (p=0.015). Finally, FGF-basic was also moderately, but significantly, predictive for ventilation status (OR: 1.04), and the upper three quartiles were also associated with ventilation (p=0.018). When considering these three cytokines together, it was observed that they clustered closely with one another in FIG. 1A.

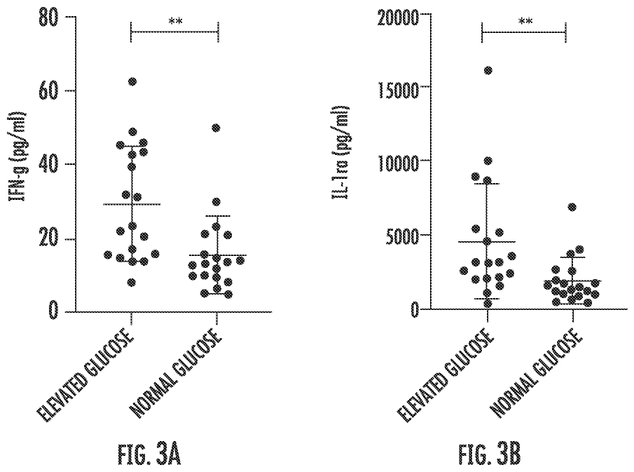

To test whether diabetes mellitus was associated with higher levels of proinflammatory cytokines, COVID-19 positive patients were separated by whether they had normal (<126 mg/dl) or elevated glucose (>126 mg/dl). Of the cytokines tested, IFN-γ (P=0.0011) and IL-1ra (p=0.0042) were significantly higher in patients with elevated glucose levels. See FIGS. 3A and 3B. To compare disease outcomes, Kaplan-Meier curves for time to ventilation after hospital admission and survival were generated. While patients with elevated blood glucose were not more likely to be put on a ventilator (p=0.87), they were less likely to survive after 28 days of diagnosis (p=0.047).

### DISCUSSION

An immunophenotype associated with future risk for ventilatory support in patients with COVID-19 was identified. Infection with SARS-CoV-2 has been associated with a dysregulated immune response, primarily characterized by elevated levels of cytokines such as IL-6, TNFa, IL-1b, and IL-10 in patients with severe disease (Liu et al., 2020; Huang et al., 2020; Chen et al., 2020). The association of these and other proinflammatory cytokines with severity has suggested the cytokine storm can be a pathologic driver of disease, and that immune responses can be targetable options for therapeutics. To observe the relationships between these proinflammatory cytokines in a COVID-19 positive patient cohort, hierarchical clustering analyses was performed of their cytokine levels.

The type 2 cytokine IL-13 is predictive of the future need for ventilation in patients with COVID-19, as well as IL-7 and FGF-basic. Notably, these three cytokines clustered together in a cytokine dendrogram, as well as significantly correlated with each other by linear regression, suggesting some common regulatory pathway or response. The proximity of IL-13 to IL-7 suggests that there is IL-7-induced type 2 Innate Lymphocytes (ILC2) or CD4+Th2 population that produces IL-13. IL-13 is often considered an anti-inflammatory cytokine, but within the lung is known to promote eosinophilia and tissue remodeling (Zhu et al., 2002). It can also increase the production of the IL-1 receptor antagonist (IL-1Ra) (Muzio et al., 1994; Scotton et al., 2005), which functions to inhibit IL-1b mediated inflammation. Additionally, IL-1Ra absolves inhibition of FGF-basic by IL-1b (Mehta et al., 2019), and can explain the close clustering between IL-13 and FGF-basic. In the examined cohort, however, IL-13 was not positively correlated with IL-1ra.

Given the interest in elevated IL-6 in patients in other studies, cytokines that clustered with IL-6 were investigated, and it was observed that IL-5 was most closely linked, suggesting an association between IL-6 and type 2 immunity, even though IL-6 itself was not associated with future need for ventilation.

Additionally, it was observed that patients with elevated blood glucose had increased IFN-γ and IL-1ra indicating a proinflammatory response. Patients with diabetes mellitus are known to have excessive inflammation and have been found to have worse outcomes due to COVID-19 (Guo et al., 2020; Zhou et al., 2020). IFN-γ is part of the Th1 immune response and an activator of macrophages, therefore it is possible that an increase in IFN-γ can indicate hyperactivation of macrophages resulting in hyperinflammation of tissues (Merad and Martin, 2020; Singh et al., 2020). IFN-γ also recruits other inflammatory cells that can induce a cytokine storm (Merad and Martin, 2020). Studies with an increased sample size can further increase understanding of the importance of IL-13 and type 2 immunity in patients with diabetes.

In summary, a previously underappreciated cytokine, IL-13, is associated with worsened outcomes from COVID-19, highlighting that type 2 immunity can be important to disease. Additionally, higher levels of proinflammatory IFN-γ were observed in patients with elevated glucose, suggesting that comorbidities can contribute to excessive inflammation. The identification of their significant predictive capabilities for severe disease increases understanding of this infection. Usage of these cytokines can also improve diagnostic capabilities, allow healthcare workers to prioritize treatment, and utilize patient-specific approaches.

### Example 2

### IL-13 Neutralization in COVID-19 Mouse Model

### Mouse Model for COVID-19 Pneumonia:

A mouse model of COVID-19 has been reported utilizing transgenic mice expressing the human ACE2 (hACE2) SARS-CoV-2 receptor (Bao et al., 2020). In these mice, human ACE2, the receptor for viral entry into host cells, is under the promoter for mouse ACE2, thereby directing human ACE2 expression to the same cells that the mouse ACE2 receptor is expressed. In this model for SARS-CoV-2 infection, mice lost roughly 10% of their body weight by day 5, had viral replication and accumulation, and exhibited immune infiltration (Bao et al., 2020).

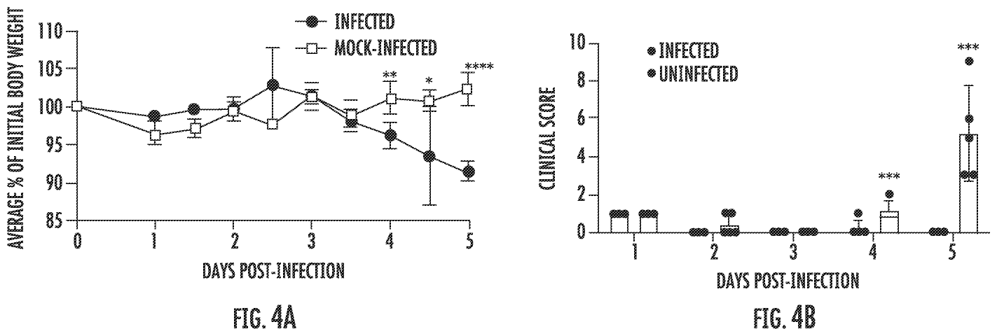

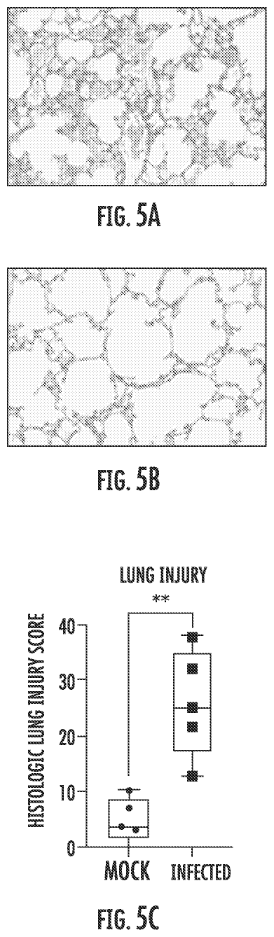

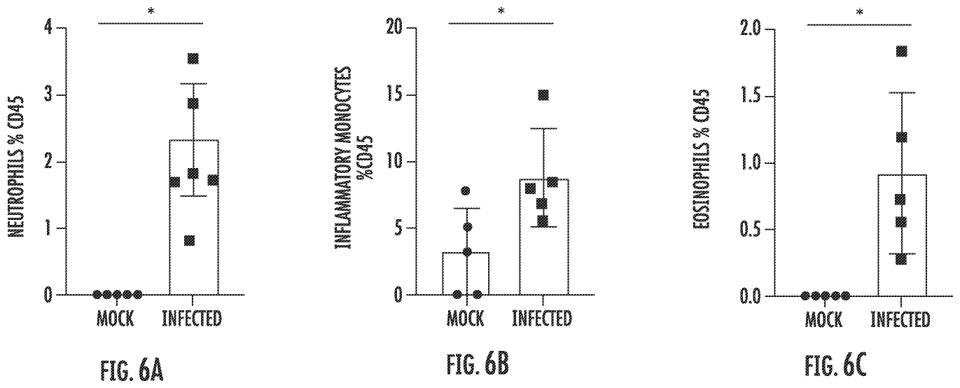

The presently disclosed study uses K18-hACE2 mice (Jackson Laboratories, Bar Harbor, Me., United States of America) for SARS-CoV-2 infection. These mice express hACE2 under the control of the keratin promoter, targeting epithelial cells for expression. Additionally, two SARS-CoV-2 strains, from the U.S. (USA-WA1/2020) and China (Hong Kong/VM20001061/2020) were utilized. Mice were infected intranasally (i.n.) with 8×10⁴ TCID50 of SARS-CoV-2 in a 50 μl inoculum volume per mouse after anesthetization with 2.5% avertin. Infection of these mice with SARS-CoV-2 led to weight loss pneumonia with interstitial infiltrate, fibrin exudate and a granulocytic and inflammatory monocyte exudative infiltration of the lungs. See FIGS. 4A, 4B, 5A-5C, and 6A-6C. Virus titers in the lung ranged from 7.5×10³ to 1.75×10⁵ PFU/ml. Thus, it was concluded that K18-hACE2 mice were permissive for SARS-CoV-2 infection and developed pneumonia characterized by interstitial and alveolar infiltration with inflammatory monocytes and granulocytes. Without being bound to any one theory, it is expected that, in humans, in addition to the predominately innate response seen in mice, an acquired cell mediated response can also occur, since pneumonia does not manifest until 2 weeks into the illness on average, a time at which acquired immunity should have developed.

IL-13 Neutralization Protects Mice from SARS-CoV-2 Infection:

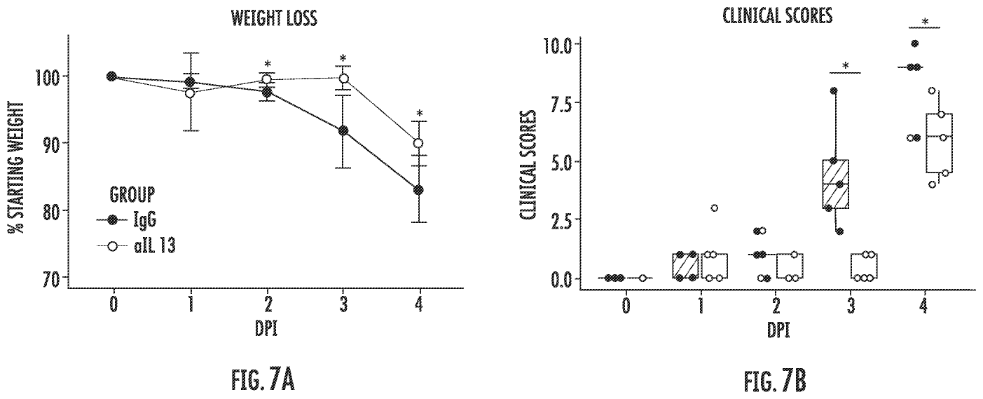

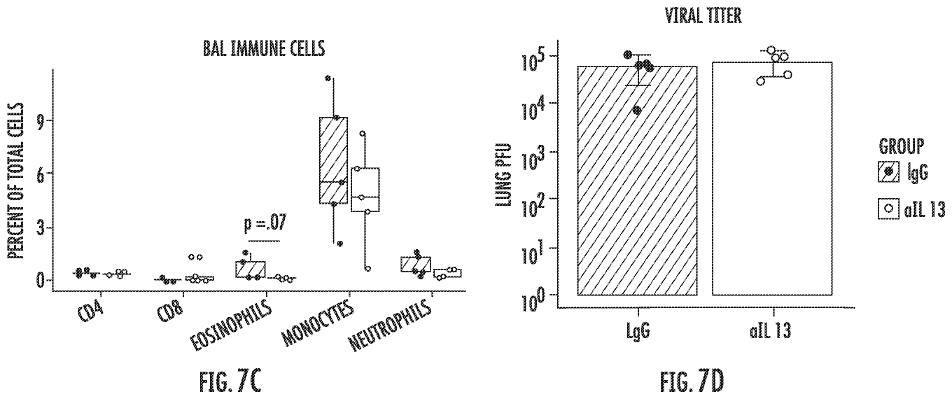

Mice were administered mAb anti-IL-13 or isotype control on day 0 and 2 of infection with SARS-CoV-2. IL-13 neutralized mice had significantly less weight loss on days 2-4 and better clinical scores on day 3 and 4. See FIGS. 7A and 7B. Mice were sacrificed on day 4 and the inflammatory infiltrate was measured in BAL fluid. IL-13-neutralized mice had less eosinophils in BAL fluid compared to mice that received an isotype control mAb (p<0.07). See FIG. 7C. SARS-CoV-2 viral load was unaffected by IL-13 neutralization. See FIG. 7D.

### Example 3

### IL-4 Elevation in Patients with Severe COVID-19

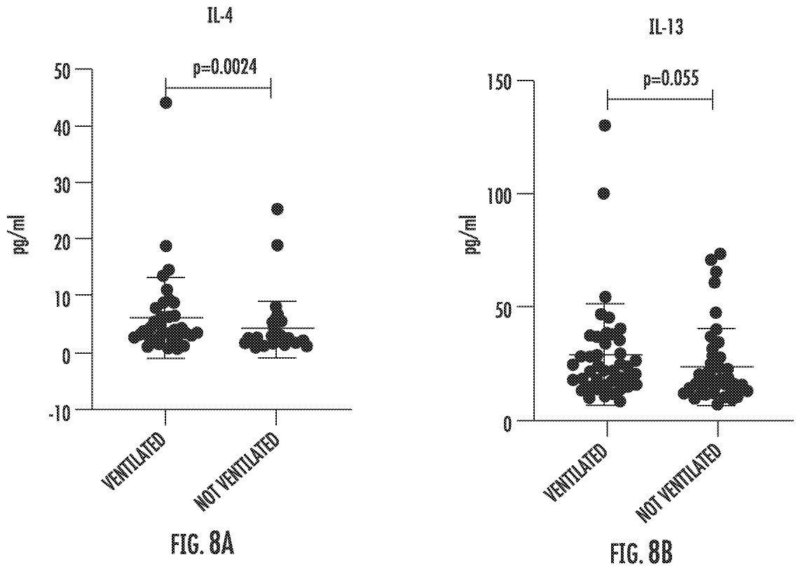

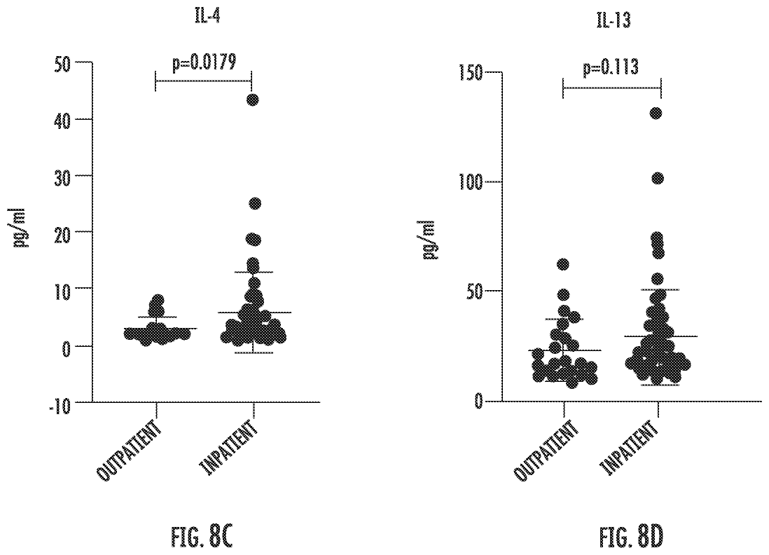

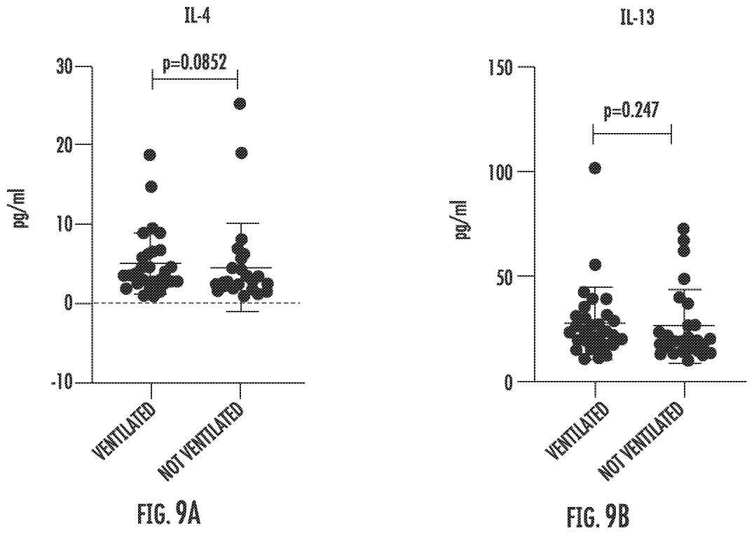

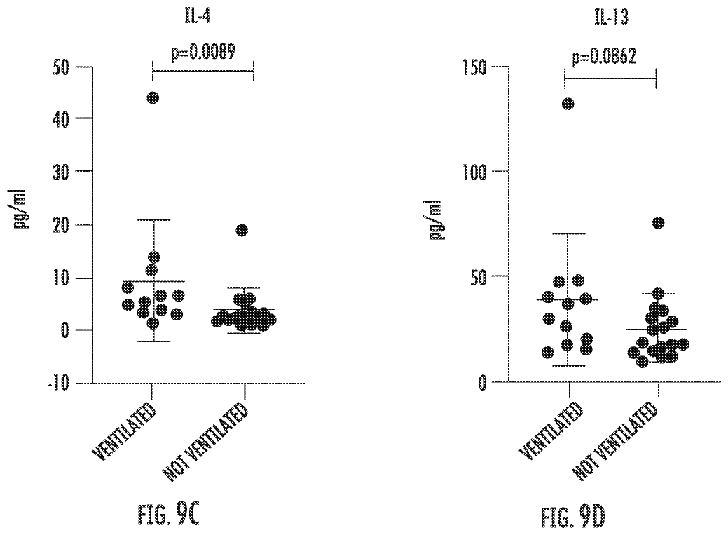

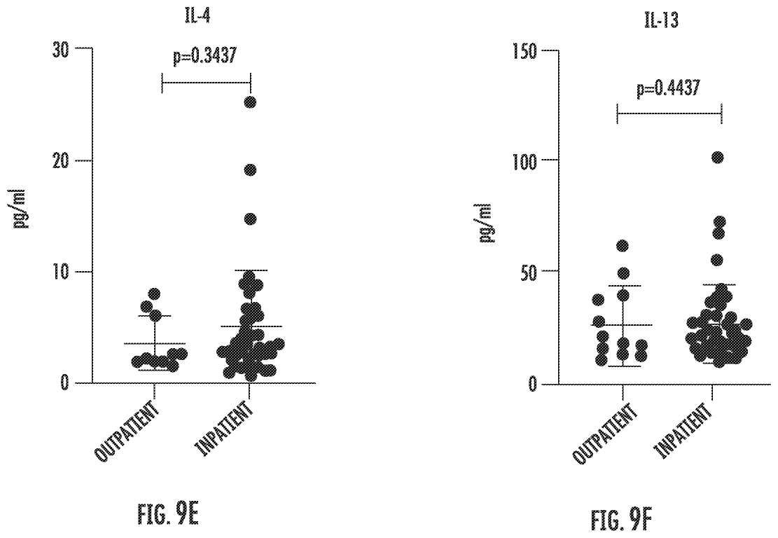

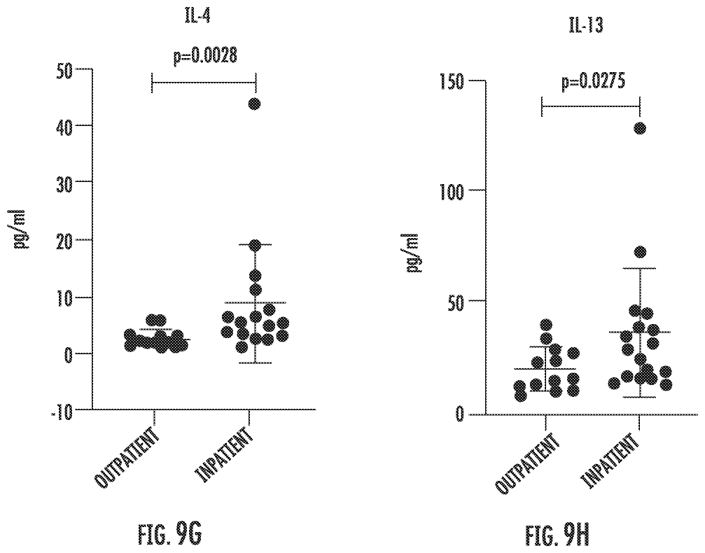

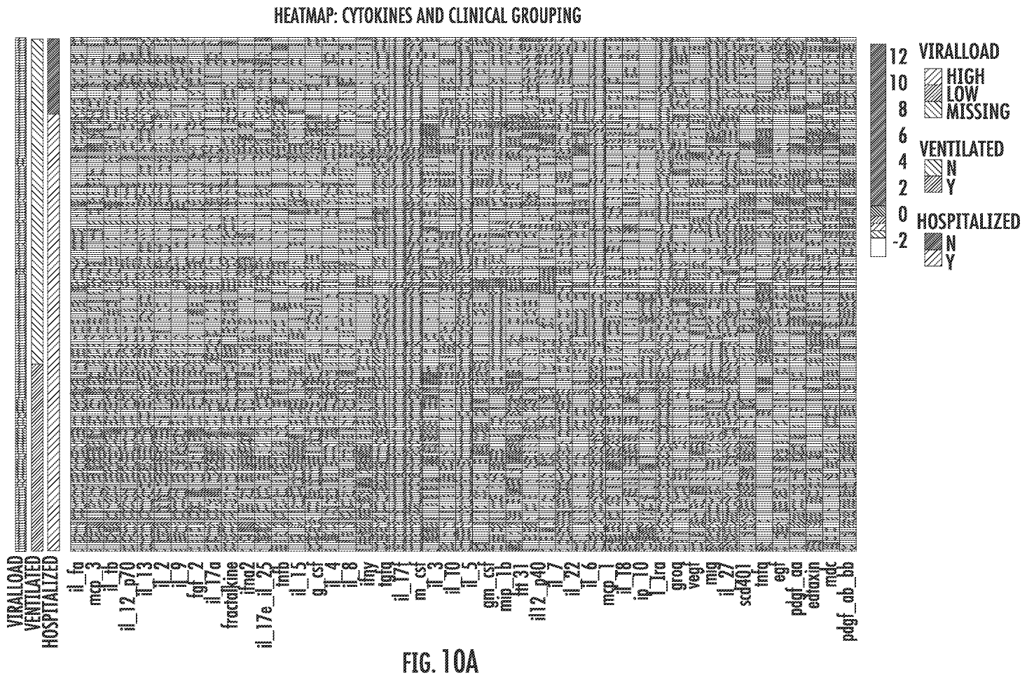

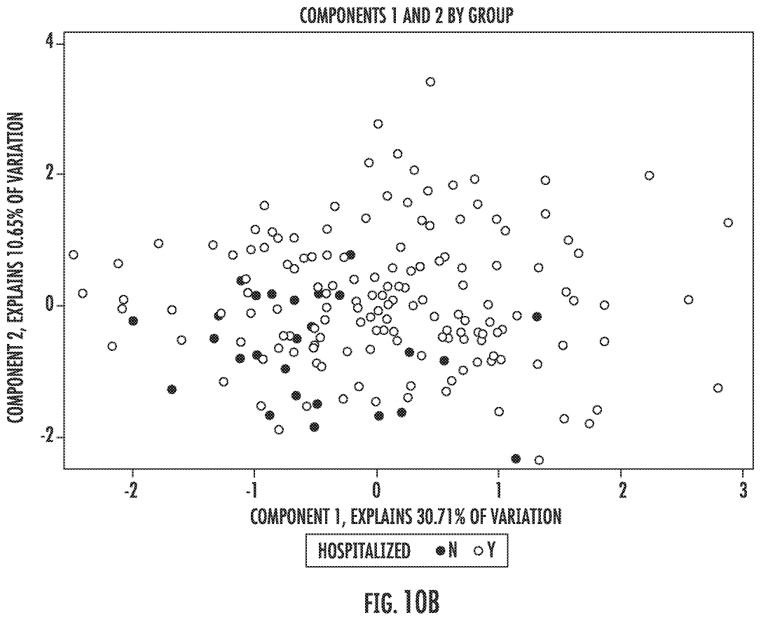

Cytokines and chemokines were measured in plasma collected acutely from a second cohort of 70 patients with COVID-19 using a MILLIPLEX® MAP Human Cytokine/Chemokine/Growth Factor Panel A (48 Plex) (Millipore Sigma, Burlington, Mass., United States of America). These patients included the patients whose plasma was assayed as described in Example 1, along with additional patients. Of the 70 patients, 21 were outpatients and 62 were hospitalized. Of the 62 hospitalized patients, 42 required mechanical ventilation. In addition to the 27 biomarkers detectable via the assay described in Example 1, the following additional biomarkers are included in the 48-plex array: sCD40L, EGF, FGF-2, Flt-3 ligand, Fractalkine, GM-CSF, GROa, IFNα2, IL-1α, IL-2, IL-3, IL-12 (p40), IL-15, IL-17A, IL-17E/IL-25, IL-17F, IL-18, IL-22, IL-27, MCP-3, M-CSF, MDC (CCL22), MIG, PDGF-AA, TGFα, TNFβ, and VEGF-A. In addition to IL-13, IL-4 is also increased in COVID-19 inpatient subjects who required mechanical ventilation. See FIGS. 8A and 8B. Levels of IL-13 and IL-4 in inpatient versus outpatient COVID-19 patients are shown in FIGS. 8C and 8D. IL-4 and IL-13 levels determined with the 48-plex array are shown for only those patients in the 70 patient cohort who were also in the smaller patient cohort of Example 1 (“overlap patients”) and for only those patients in the 70 patient cohort who were not in the smaller cohort of Example 1 (“unique patients”). See FIGS. 9A-9H.

### Example 4

### Expanded Cohort and Dupilumab Studies

Discarded human plasma samples from COVID-19 positive and negative patients at the University of Virginia Medical Center were used for cytokine and growth factor analyses. The collection of biological specimens and de-identified patient information was approved by the University of Virginia Institutional Review Board (IRB-HSR #22231 and 200110). In mice, neutralizing antibodies or isotype control were used to assess the role of IL-13 during COVID-19. All mouse work was approved by the University of Virginia Institutional Animal Care and Use Committee, and all procedures were performed in the University certified animal Biosafety Level Three laboratory.

### Patients:

Patients with a positive real time-quantitative polymerase chain reaction (RT-qPCR) test for COVID-19 at the Clinical Microbiology Laboratory at the University of Virginia Medical Center had any remnant EDTA-plasma samples within 48 hours of the time of diagnosis or hospitalization collected. EDTA-plasma from 178 patients were analyzed in this study. Blood was centrifuged at 1300×g for 10 minutes, then plasma was aliquoted and stored at −80° C. until testing.

Demographics (age, gender, race), comorbidities, hospitalization status, lab results, and other clinical information were obtained from the electronic medical record (EMR). See Table 1, below. Severity of COVID-19 illness was assessed through review of the electronic medical record in two ways: first by inpatient admission vs outpatient care, and second by the use of supplemental oxygen (none vs any supplemental oxygen, and supplemental oxygen delineated as low flow nasal canula vs mechanical ventilation or high flow oxygen). In addition, supplemental oxygen was scored as occurring at the time of the blood draw or at a future time. Days from symptom onset was scored as previously described (Lucas et al., 2020) based on the patient's determination or by the earliest reported symptom from the patient as recorded in the electronic medical record.

TABLE 1
Age, Sex and Clinical Status of UVA Patients

| | Outpatient (n = 26) | Inpatient (n = 152) |
|---|---|---|
| Age (mean +/− SD) | 42.4 (15.1) | 59.2 (16.7) |

Sex

| | | |
|---|---|---|
| Male | 11 (42.3%) | 66 (43.4%) |
| Female | 15 (57.7%) | 86 (56.6%) |

Race

| | | |
|---|---|---|
| Caucasian | 5 (19.2%) | 60 (39.5%) |
| African-American | 7 (26.9%) | 34 (22.4%) |
| Asian | 2 (7.7%) | 3 (2.0%) |
| Other | 12 (46.1%) | 55 (36.2%) |

Ethnicity

| | | |
|---|---|---|
| Hispanic | 13 (50.0%) | 61 (40.1%) |
| Non-Hispanic | 13 (50.0%) | 91 (59.9%) |
| Timing of blood draw | 4.40 (2.95) | 11.3 (11.2) |
| from day of symptom | | |
| onset (mean SD) | | |
| Missing/unknown | 6 (23.1%) | 40 (26.3%) |

Respiratory Status at time of Blood Draw

| | | |
|---|---|---|
| No oxygen requirement | 25 (96.2%) | 45 (29.6%) |
| Supplemental oxygen only | 1 (3.8%) | 57 (37.5%) |
| Mechanical ventilation | 0 | 50 (32.9%) |

At any time during illness

| | | |
|---|---|---|
| No oxygen requirement | 25 (96.2%) | 28 (18.4%) |
| Supplemental oxygen only | 1 (3.8%) | 59 (38.8%) |
| Mechanical ventilation | 0 | 65 (42.8%) |
| Ct value (mean, SD) | 28.2 (9.22) | 27.2 (6.15) |
| Missing/Unknown | 11 (42.3%) | 113 (74.3%) |
| Comorbidity | 7 (26.9%) | 101 (66.4%) |
| Diabetes | 5 (19.2%) | 66 (43.4%) |
| Cancer | 1 (3.8%) | 14 (9.2%) |
| Immunosuppression | 0 (0%) | 11 (7.2%) |
| Kidney Disease | 0 (0%) | 30 (19.7%) |
| Heart Disease | 1 (3.8%) | 28 (18.4%) |
| Lung Disease | 1 (3.8%) | 22 (14.5%) |
| Liver Disease | 0 (0%) | 2 (1.3%) |
| Stroke | 1 (3.8%) | 13 (8.6%) |

Cytokine concentrations in plasma were measured using the nILLIPLEX® MAP Human Cytokine/Chemokine/Growth Factor Panel A (48 Plex) (Millipore Sigma, Burlington, Mass., United States of America). Cytokines detected were sCD40L, EGF, Eotaxin, FGF-2, Flt-3 ligand, Fractalkine, G-CSF, GM-CSF, GROα, IFNα2, IFNγ, IL-1α, IL-1β, IL-1ra, IL-2, IL-3, IL-4, IL-5, IL-6, IL-7, IL-8, IL-9, IL-10, IL-12 (p40), IL-12 (p70), IL-13, IL-15, IL-17A, IL-17E/IL-25, IL-17F, IL-18, IL-22, IL1-27, IP-10, MCP-1, MCP-3, M-CSF, MDC (CCL22), MIG, MIP-1α, MIP-1β, PDGF-AA, PDGF-AB/BB, TGFα, TNFα, TNFβ, and VEGF-A (RANTES was excluded).

As a test of validation of the results from the University of Virginia Hospital, an additional 45 patients with symptomatic COVID-19 (all inpatients) from Virginia Commonwealth University Medical Center were analyzed. IL-13 was measured in EDTA plasma from these patients using the Pro Human Cytokine 27-plex Assay (R & D Systems, Minneapolis, MVinnesota, United States of America). Demographics of the patients from Virginia Commonwealth University (VCU) are shown in Table 2, below.

TABLE 2
Age, Sex and Clinical Status of VCU patients.

| | Non-Severe (n = 19) | Severe (n = 27) |
|---|---|---|
| Age (mean +/− SD) | 50.4 (14.6) | 60.3 (9.04) |

Sex

| | | |
|---|---|---|
| Female | 9 (47.4%) | 9 (33.3%) |
| Male | 10 (52.6%) | 18 (66.7%) |

Race

| | | |
|---|---|---|
| Caucasian | 2 (10.5%) | 7 (25.9%) |
| African-American | 14 (73.7%) | 19 (70.4%) |
| Asian | 1 (5.3%) | 0 (0.0%) |
| Other | 2 (10.5%) | 1 (3.7%) |

Ethnicity

| | | |
|---|---|---|
| Hispanic | 2 (10.5%) | 1 (3.7%) |
| Non-Hispanic | 17 (89.5%) | 26 (96.3%) |

### Database and Inclusion Criteria for Dupilumab:

Data for a cohort of 350,004 COVID-19 patients were retrieved from the COVID-19 Research Network provided by TriNetX (Cambridge, Mass., United States of America). At the time of data retrieval, the network included data for 400 million patients from 130 health care organizations in 30 countries. COVID-19 patients were identified via the ICD-10 code U07.1 or the presence of a SARS-CoV-2-related RNA diagnosis within the last eleven months. Propensity scores matched cohorts 1:1 using a nearest neighbor greedy matching algorithm with a caliper of 0.25 times the standard deviation. Outcomes were defined as ventilation assist and death. Measures of association including risk differences with their respective 95% CI's were calculated. In addition, Kaplan-Meier curves were generated for each analysis.

A sub cohort with indications for Dupilumab use was generated using ICD-10 codes for asthma (J45), atopic dermatitis (L20.8), and pansinusitis (J01.40 and J32.4). Drug use was identified via RxNorm codes for Dupilumab (1876376) and the lab value for C-reactive protein (9063). Patients receiving Dupilumab are on average one year older.

### N3C Dupilumab Analysis:

Deidentified data in the National Cohort Collaborative Cohort (N3C) enclave, which currently contains 2 years of medical record data from 34 United States sites was utilized to explore association between Dupilumab use and COVID-19 outcomes. This enclave represents >2 million persons (including ˜300,000 COVID-19 cases) and medical facts from more than ˜90 million visits. Values less than 20 have been suppressed as per current N3C publication policy.

### Cohort Definitions

1. Dupilumab+: If Patient had Dupilumab within 61 days prior to their first COVID-19 diagnosis date
2. Controls+: Patients with no record of Dupilumab within 2 months prior to their first COVID-19 diagnosis date.

### Outcome Definitions

1. Hospitalized: Patients who became inpatient within 6 weeks post COVID-19 diagnosis date.
2. Death: If subjects death date is after their first COVID-19 diagnosis date.
3. With Ventilation: If Patients put on ventilator within 6 weeks of any of their COVID-19 diagnosis dates (window is double sided: procedure could have been 6 weeks before or after any of their diagnosis dates).

The incidence of COVID positivity in people on dupilumab [cohort definition 1 above/(definition 1+2)], along with 95% confidence intervals, was calculated. Then, a case-control design was used. Dupilumab+patients were matched to control+patients in a 1:5 ratio, with exact matching on gender, race, ethnicity, N3C site, asthma and nearest matching on age. Conditional logistic regression was used to compare COVID-19 severity outcomes within this matched subset of COVID+patients. A sensitivity analyses was performed excluding asthma from the matching criteria.

### Virus and Cell Lines:

SARS-Related Coronavirus 2 (SARS-CoV-2), isolate Hong Kong/VM20001061/2020 (NR-52282) was obtained from the Biodefense and Emerging Infections Research Resources Repository (BEI Resources), National Institute of Allergy and Infectious Diseases (NIAID), National Institutes of Health (NIH). Virus was propagated in Vero C₁₀₀₈, Clone E6 (American Type Culture Collection (ATCC), Manassas, Va., United States of America; CRL-1586) cells cultured in Dulbecco's Modified Eagle's Medium (DMEM, Gibco 11995040, Thermo Fisher Scientific, Waltham, Mass., United States of America) supplemented with 10% fetal bovine serum (FBS) and grown at 37° C., 5% C₀₂. Initial viral stocks were used to infect Vero E6 cells, generating passage 1 (P1) stocks. These P1 stocks were then used to infect additional Vero E6 cells, generating passage 2 (P2) stocks, which were used for all experiments.

### Viral Propagation:

Vero E6 cells grown to 90% confluency in T75 tissue culture flasks (Thermo Fisher Scientific, Waltham, Mass., United States of America) were infected with SARS-CoV-2 at a multiplicity of infection of 0.025 in serum-free DMEM. Vero E6 cells were incubated with virus for two hours at 37° C., 5% C₀₂, after which the virus was removed, media was replaced with DMEM supplemented with 10% FBS, and flasks were incubated at 37° C., 5% C₀₂. After two days, infected flasks showed significant cytopathic effects, with >50% of cells unattached. Cell supernatants were collected, filtered through a 0.22 m filter (Millipore, SLGP003RS; Millipore Sigma, Burlington, Mass., United States of America), and centrifuged at 300×g for ten minutes at 4° C. Cell supernatants were divided into cryogenic vials (Corning, 430487; Corning, Inc., Corning, N.Y., United States of America) as viral stocks and stored at −80° C. until use.

### Challenge:

8-16 week-old male Tg(K18-hACE2) 2Prlmn (Jackson Laboratories, Bar Harbor, Me., United States of America) mice were challenged with 5000 μlaque forming units (PFUs) of SARS-CoV-2 in 50 μl by an intranasal route under ketamine/xylazine sedation. Mice were followed daily for clinical symptoms, which included weight loss (0-5), activity (0-3), fur appearance and posture (0-2), and eye closure (0-2). Mice were given 150 μg of anti-IL-13 (clone eBiol316H; cat #16-7135-85; Thermo Fisher Scientific, Waltham, Mass., United States of America) or an isotype matched control IgG (clone eBRG1; #16-4301-85; Thermo Fisher Scientific, Waltham, Mass., United States of America) administered on day 0, 2, and 4 post infection.

### Viral Titers:

The left lobe of the lung was removed, placed in a disposable tissue grinder with 1 ml of serum-free DMEM on ice, and then ground. Lung homogenates were centrifuged at 300×g for 10 minutes, and then the supernatants were collected and frozen at −80° C. until use. Plaque assays were as described previously (Moreau et al., 2020). Briefly, Vero E6 cells seeded in 12-well tissue culture plates were infected with lung homogenates serially diluted in serum-free DMEM. Plates were then incubated at 370 C, 5% C₀₂ for two hours to allow viral infection of the cells, before washing with sterile PBS (Gibco 10010⁻⁰²³, Thermo Fisher Scientific, Waltham, Mass., United States of America) to remove virus and replacing with an overlay of DMEM, 2.5% FBS containing 1.2% Avicel PH-101 (Sigma Aldrich, St. Louis, Mo., United States of America). After incubation at 37° C., 5% C₀₂ for two days the overlay was removed, wells were fixed with 10% formaldehyde and stained with 0.1% crystal violet to visualize plaques and calculate viral titers as PFU/ml.

### Histology:

Tissues were fixed in formaldehyde, processed and embedded in paraffin. Sections were stained with H&E, Masson's Trichrome and Periodic Acid-Schiff using standard protocols. Slides were scanned at 20× magnification. Histopathological scoring for lung tissue was done according to the guidelines of the American Thoracic Society (Leist et al., 2020).

For antibody immunostaining lung sections were deparaffinized and heat-mediated antigen retrieval performed using Tris-EDTA buffer (10 mM Tris Base, 1 mM EDTA, 0.05% Tween-20 pH 8.0; incubation 20 min 95° C.). Non-specific protein was blocked (2% donkey serum, 1% BSA, 0.05% Tween-20) prior to blocking endogenous avidin and biotin (Thermo Fisher Scientific, Waltham, Mass., United States of America). Lung sections were incubated with primary antibodies (anti-Ym1 (goat polyclonal antibody, biotinylated, 1:100 dilution, BAF2446, R & D Systems, Minneapolis, Minn.) or anti-RELMa (rabbit polyclonal antibody, 1:100 dilution, 500-P214, PeproTech, Inc., Rocky Hill, N.J., United States of America)) overnight at 4° C., washed in PBS containing 0.05% Tween-20 before incubation with secondary antibodies (Streptavidin 557, 1:800 dilution, NL999, R & D Systems, Minneapolis, Minn., United States of America; Donkey anti-rabbit IgG 637, NL005, R & D Systems, Minneapolis, Minn., United States of America) for 1 hour at room temperature followed by mounting with DAPI containing fluoromount (Southern Biotech, Birmingham, Ala., United States of America). Images were captured with an EVOS FL imaging system (Thermo Fisher Scientific, Waltham, Mass., United States of America). Analysis of images was performed using ImageJ software (version 2.09.0-rc69/1.52p) on sections where sample identification was blinded for the investigator. For calculation of antibody positive staining background autofluorescence was subtracted from all images based on pixel intensities of sections stained with secondary antibodies only. Analysis was limited to region of interest (airway, vessels or parenchyma), a threshold was applied (by eye) to all images to incorporate positively stained pixels and area of positively stained pixels within the region of interest calculated. For measurements of airway positivity, stain intensity was normalized to the length of the basement membrane.

### Mouse Bronchioalveolar Lavage:

BAL fluid was collected from each animal through cannulation of the exposed trachea, and flushing twice with 0.7 mL of PBS. BALF samples were centrifuged at 500×G for 5 minutes, and supernatant was immediately frozen for later cytokine analyses. For flow cytometry, pelleted cells were resuspended in FACS (PBS+5% FBS) buffer for staining and with Zombie NIR (Biolegend, 423105, SanDiego, California, United States of America), CD45, Alexa Fluor 532 (eBioscience, 58-0459-42, San Diego, Calif., United States of America), CD11c, PE-Cy7 (Biolegend, 117317, San Diego, Calif., United States of America), CD11b, BV480 (BD Biosciences, 566117, San Jose, Calif., United States of America), SIGLEC F, AF700 (eBioscience, 56-1702-80, San Diego, Calif., United States of America), Ly-6c, FITC (Biolegend, 128005, San Diego, Calif., United States of America), and Ly-6G, BV650 (Biolegend, 127641, San Diego, Calif., United States of America) and then fixed in IC-fixation buffer (eBioscience, 00-8222-49, San Diego, Calif., United States of America). Samples were run on a flow cytometer sold under the tradename CYTEK™ Aurora Borealis (Cytek Biosciences, Fremont, Calif., United States of America). Neutrophils are Zombie NIR−, CD45+, CD11C−, CD11B+, and Ly-6G+; eosinophils are Zombie NIR−, CD45+, CD11C−, CD11B+, and Siglec-F+; inflammatory monocytes are Zombie NIR-, CD45+, CD11C−, CD11B+, Ly-6G-, and Ly-6C+.

Cytokine analyses were performed via mouse 32-plex chemokine/cytokine assay (MCYTMAG-70K-PX32, Millipore Sigma, Burlington, Mass., United States of America). Samples were run following manufacturers protocol after an 18-hour incubation before being run on an analyzer sold under the tradename MAGPIX™ (Luminex Corporation, Austin, Tex., United States of America).

### RNA Seq:

RNA was extracted from murine lung tissue preserved in trizol by bead beating (tissuelyserII, Qiagen, Hilden, Germany), followed by a phenol-chloroform extraction and RNA Isolation Kit (Qiagen, Hilden, Germany). RNA quality was assessed using Agilent Tape Station RNA kit (Agilent Technologies, Santa Clara, Calif., United States of America). Library prep, sequencing and primary data analysis was performed by the Genome Analysis and Technology Core, RRID:SCR_018883. Briefly, ribosomal RNA was depleted using the rRNA depletion kit (NEB E6310, New England Biolabs, Ipswich, Mass., United States of America). Following rRNA depletion cDNA libraries were prepared using the NEB ultra-directional library preparation kit 2.0 (NEB E7760, New England Biolabs, Ipswich, Mass., United States of America) and indexed using a primer set sold under the tradename NEBNEXT™ Multiplex Oligos for Illumina (NEB E7335, E7500, New England Biolabs, Ipswich, Mass., United States of America). Library size and purity were verified using the Agilent Tape Station D5000 High Sensitivity (HS) assay kit (Agilent Technologies, Santa Clara, Calif., United States of America). Library concentration was measured with a qubit DNA HS assay (Invitrogen Corporation, Waltham, Mass., United States of America). Libraries concentrations were normalized and 15 libraries were multiplexed per run. Diluted libraries were sequenced on the Illumina Nextseq 500 sequencing system (Illumina Corporation, San Diego, Calif., United States of America) using a 150 high output kit (400 Million reads, 2×75 bp paired end, 150 cycles).

### RNA Seq Data Analysis:

RNA-seq reads were first processed using Cutadapt (Martin, 2011) to trim the adapter sequences and then the quality of the reads was assessed by FastQC (Babraham Bioinformatics, 2019) and MultiQC (Ewels et al., 2016). After these processes the reads were aligned to the mouse Ensembl GRCh38.76 primary assembly using STAR v2.5.3a (Dobin and Gingeras, 2015) in a two-passing mode to generate gene matrix for differential gene expression. Differentially expressed genes were determined using the DESeq2 package (Love et al., 2014) Rstudio (online at rstudio.com).

Enrichment analysis was applied to the total gene counts using the reactome. GSA package (Griss et al., 2020) in Rstudio. Enrichment analysis was applied to the total gene counts using the CAMERA algorithm (Wu and Smyth, 2012).

### Statistical Methods:

For the clinical study, a total of 47 cytokines, chemokines and growth factors were measured in 178 COVID-19 positive patients with blood samples. For cytokines of interest, levels at the time of COVID-19 diagnosis or admission were compared between patients with different severity of illness using a Mann-Whitney U test. For hierarchical clustering, the pheatmap function in the pheatmap library was used (R— project.org). Cytokines were scaled by row (patients), and the clustering was calculated using the complete linkage method. Statistical analyses were performed using GraphPad Prism and R.

Since these cytokine measurements were highly correlated, principal component analysis (PCA) was performed to identify distinct features among correlated cytokines, and thus reduce the dimensionality. Due to their skewed distributions and variable scales, the cytokines were log-transformed first, and then standardized with mean of zero and standard deviation of one. The first five principal components captured 62% of total variation in the cytokines. For each principal component, those cytokines with loading score of 0.5 or above were retained, showing the strength of their influences within the component. Additionally, the network analysis using the qgraph package in R was performed to characterize the complex structural relationships among cytokine measurements and optimized with graphical LASSO. The nodes represented individual cytokines, and edges represented their correlations in that highly correlated cytokines were connected closer with thick edges.

Survival Analysis was performed for the time to ventilation from symptom onset. Those who were not ventilated were censored at 40 days. Patients were classified into 4 quartiles based on the cumulative distribution of IL-13 levels, and the probabilities of ventilation were estimated by the Kaplan-Meier method. Since the two upper quartiles had statistically identical results in the preliminary analysis, they were combined in the final log-rank test and Cox regression done for their relative performance over the lower quartiles, and were corrected for sex, age, and cumulative number of comorbidities (including: diabetes, cancer, stroke, and heart, liver, kidney or lung diseases).

For Dupilumab study, TriNetX analytics tools (TriNetX, Cambridge, Mass., United States of America) were used to obtain baseline characteristics, balance cohorts with propensity score matching and analyze outcomes of interest in the final cohorts. Baseline characteristics, including demographics, diagnoses, procedures, and medication were obtained. Propensity score matching was used to balance cohorts. Propensity scores matched cohorts 1:1 using a nearest neighbor greedy matching algorithm with a caliper of 0.25 times the standard deviation. Outcomes were defined as ventilation assist and death. Measures of association including risk differences with their respective 95% CI's were calculated. In addition, Kaplan-Meier curves was generated.

For clinical scores, weight loss, and scored histological sections of mouse study, a two-tailed Student's t test was used to determined statistical significance. For IHC sections with multiple quantified images per mouse or human tissue, response differences between groups (e.g., infected vs. uninfected, or IgG vs aIL-13) were evaluated in the mixed-effects model to account for within-individual correlation. P value<0.05 was considered significant.

### Results:

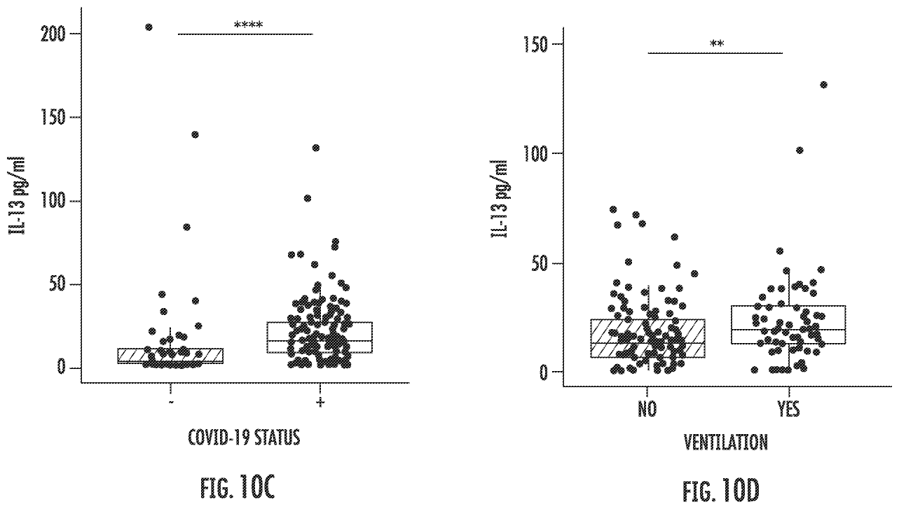

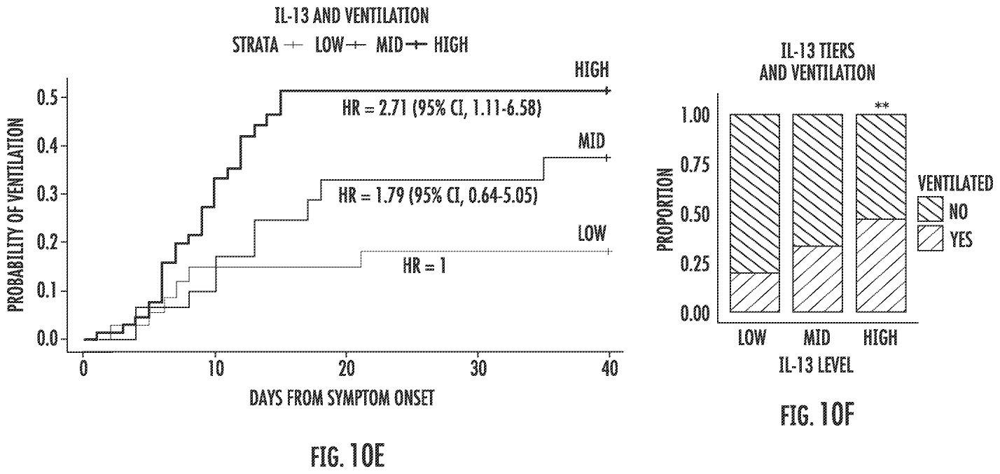

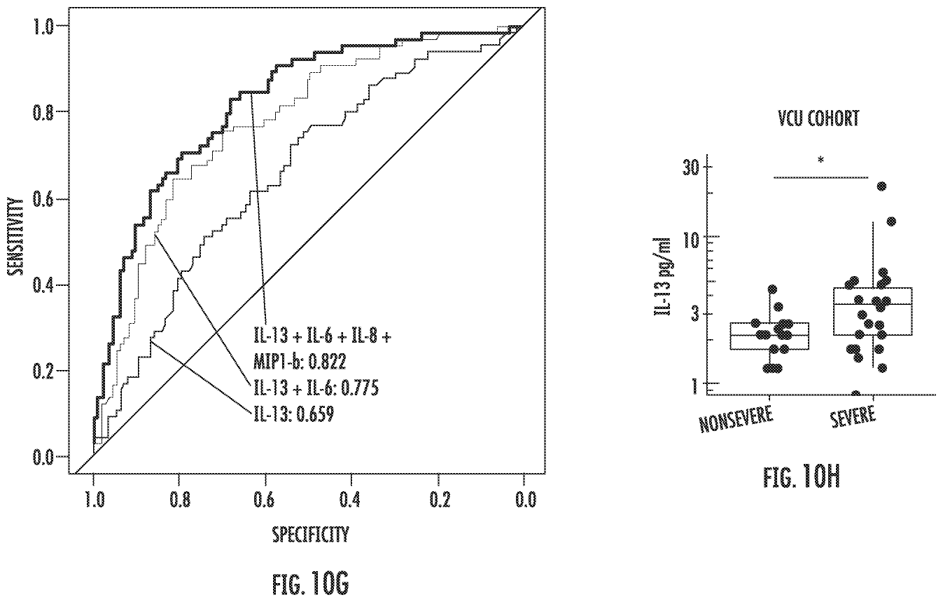

Plasma cytokines were analyzed in 178 patients with COVID-19, 26 of whom received their care as outpatients and 152 as inpatients. See Table 1, above. Cytokines were measured in the blood sample taken closest to the first positive COVID-19 RT-qPCR test. To understand the potential interrelationships between the different cytokines measured in our cohort, a heatmap was generated for patients, grouped by hospitalization status and cytokines. See FIG. 10A. Cytokines were arranged by principal component 1 from the scatterplot comparing principal component 1 and 2 from principal component analysis (PCA). See FIG. 10B. Of note, there was no obvious separation at the sampled timepoint between patients who were ventilated compared to those who were not, highlighting a heterogeneous cytokine response between patients.

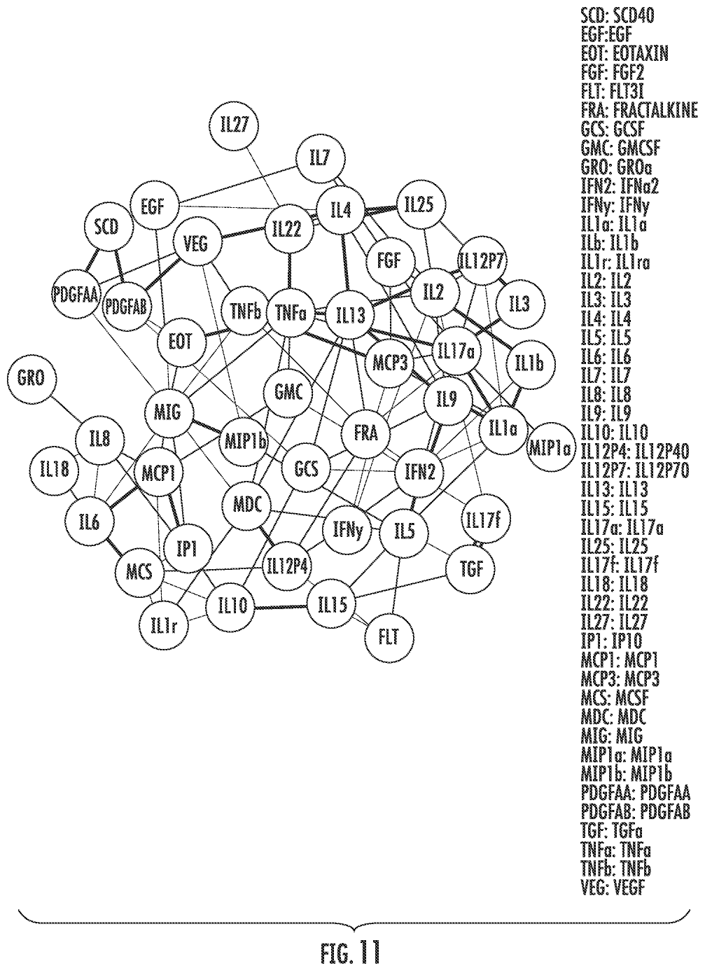

The scatterplot of PC1 and PC2 from a principal coordinate analysis (PCA) (see FIG. 10B) showed separation, albeit with overlap, of outpatients, with less severe disease (black dots) from 8 inpatients (grey dots). IL-13 was in the top 6 of the 47 cytokines/growth factors in PC1 of the PCA. See Table 3, below. This is of interest because the components estimated via PCA are able to retain information, separating patients by disease severity (inpatient vs outpatient). IL-13's position as a high-ranking contributor to PC1 suggested its overall importance in the disease process. Additionally, network analysis of all of the cytokines measured showed the close relationship between IL-13 and other type 2 cytokines identified in PC1, including IL-9 and IL-25. See FIG. 11.

TABLE 3
Contributors to Component 1 in PC A Plot.
Principal Component 1

| Order | Cytokine | PC.1.Loading |
|---|---|---|
| 1 | IL-1a | 0.86636 |
| 2 | MCP-3 | 0.82474 |
| 3 | IL-1b | 0.79245 |
| 4 | IL-12p70 | 0.79093 |
| 5 | MIP-1a | 0.78886 |
| 6 | IL-13 | 0.77694 |
| 7 | IL-2 | 0.76816 |
| 8 | IL-9 | 0.75838 |
| 9 | FGF2 | 0.75743 |
| 10 | IL-17a | 0.73832 |
| 11 | Fractalkine | 0.72882 |
| 12 | IFNa2 | 0.71946 |
| 13 | IL-25 | 0.71022 |
| 14 | TNF-b | 0.70035 |
| 15 | IL-15 | 0.67703 |

IL-13 is implicated in numerous processes, including (i) recruitment of eosinophils and M2 macrophages to the lung, (ii) induction of mucus secretion into the airways and goblet cell metaplasia, (iii) proliferation of smooth muscle cells, and (iv.) fibrosis via fibroblast activation and subsequent collagen deposition (Chiaramonte et al., 2001; and Marone et al., 2019). Plasma levels of IL-13 are significantly higher in COVID-19 positive patients compared to uninfected patients. See FIG. 10C. Moreover, in the present studies, it was found that plasma IL-13 levels are significantly elevated in patients who required mechanical ventilation. See FIG. 10D. Additionally, when stratified into three IL-13 level expression groups, patients in the higher-expressing level tiers were more likely to be ventilated and the risk of ventilation was 2.7 times for those in the top tier compared to those in the lowest tier (HR 2.71; 95% CI, 1.11-6.58). See Figures l0E and 10F. To determine whether this association with IL-13 and disease severity was not because patients who required ventilation had elevated cytokines due to a longer duration of illness by the time their sample was taken, a linear regression between IL-13 and days from symptom onset to blood draw was generated and no significant correlation was observed. However, IL-13 μlasma levels were observed to increase from patients' initial to secondary blood draw, on average, suggesting levels of IL-13 could be increasing within an individual patient's disease course. To assess the ability of IL-13, alone or in combination with other cytokines, to be able to predict ventilation outcomes in patients, a receiver operating characteristic (ROC) analysis was performed. IL-13 alone performed modestly (area under the ROC curve [AUC]=0.659). Inclusion of the cytokine IL-6 increased the predictive capability (AUC=0.775), and additional inclusion of IL-8 and MIP-β further improved the model (AUC=0.822). See FIG. 10G. To validate results from the University of Virginia Hospital, cytokines from an additional 48 inpatients with COVID-19 from Virginia Commonwealth University Medical Center were analyzed. IL-13 levels measured in plasma were found to be elevated in the hospitalized patients who received oxygen via high flow nasal canula or mechanical ventilation compared to inpatients who did not. See FIG. 10H.

To test the contribution of IL-13 to respiratory failure in COVID-19, a K18-hACE2 transgenic mouse model of COVID-19 was utilized (Moreau et al., 2020; Rathnasinghe et al., 2020; and Winkler et al., 2020b). In this model, mice progress to severe disease starting at day five post-infection (pi) with SARS-CoV-2, with most mice succumbing to disease by day seven or eight.

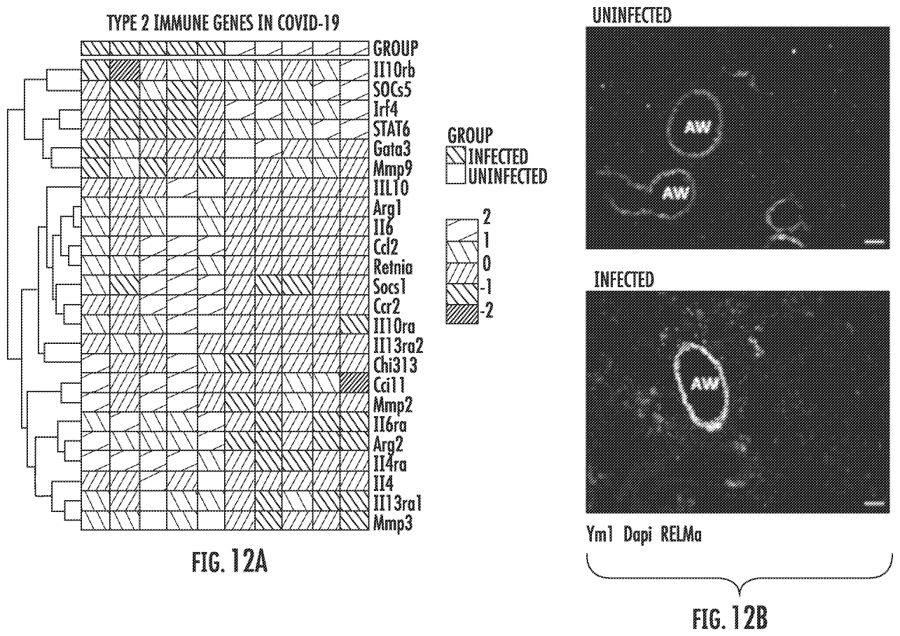

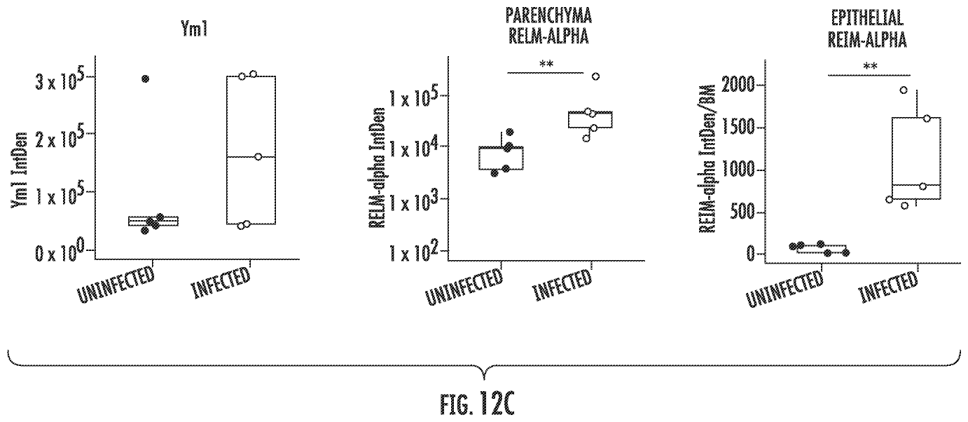

The impact of SARS-CoV-2 infection was characterized in the mouse lung. Reactome analysis of differentially regulated genes from whole tissue RNAseq revealed a significant enrichment of genes involved in IL-4 and IL-13 signaling in infected lung (FDR adjusted p-value=0.03) on day five post infection. See Table 4, below. Il4ra and Il13ral were upregulated, indicating the potential for increased signaling even in the absence of detectable increases in cytokine expression. Additional type 2 immune effectors known to be regulated by IL-4 and IL-13, including Chil3, Retnla, Ccl11 and Arg1, were impacted by SARS-CoV-2 infection. See FIG. 12A.

In support of enhanced type 2 associated genes, protein expression of Ym1 (Chil3) and RELMα (Retnla) was increased in the lungs following infection (see FIGS. 12B and 12C), as measured by immunohistochemistry (IHC). Together, this highlighted that type 2 immunity was induced in the lungs due to infection. Of note, IL-4 and IL-13 share a receptor subunit and induce common pathways, so it is difficult to delineate their respective contributions to the upregulation of type 2 effector genes (Winkler et al., 2020b). However, because an association of IL-13 with severe disease was observed in patients, it is believed that IL-13 signaling in the lung following infection could be contributing to worse outcomes.

TABLE 4
Enriched Pathways in Differentially Regulated Genes in Murine COVID-19 Infection.

| | Entities | Entities | Entities | Entities | | Rxns | Rx ns | Rxns | |
|---|---|---|---|---|---|---|---|---|---|
| Pathway Name | Found | Total | Ratio | Pval | Entities FDR | Found | Total | Ratio | RNA seq |
| Signaling by Interleukins | 581 | 647 | 0.044 | 0.00154 | 0.0291 | 489 | 493 | 0.038 | ▴ |
| Deubiquitination | 259 | 288 | 0.020 | 0.00125 | 0.0243 | 77 | 77 | 0.006 | ▴ |
| Interleukin-4 and Interleukin-13 signaling | 187 | 216 | 0.015 | 0.00168 | 0.0312 | 46 | 47 | 0.004 | ▴ |
| Antiviral mechanism by IFN-stimulated | 81 | 94 | 0.006 | 0.00129 | 0.0250 | 27 | 31 | 0.002 | ▴ |
| genes | | | | | | | | | |
| FCGR3A-mediated IL10 synthesis | 45 | 141 | 0.010 | 0.00138 | 0.266 | 20 | 20 | 0.002 | ▾ |
| Aquaporin-mediated transport | 44 | 68 | 0.005 | 0.00172 | 0.0318 | 24 | 25 | 0.002 | ▾ |
| Antimicrobial peptides | 40 | 123 | 0.008 | 0.00161 | 0.0302 | 39 | 58 | 0.004 | ▴ |
| Glucagon signaling in metabolic regulation | 32 | 40 | 0.003 | 0.00181 | 0.0329 | 6 | 6 | 0.000 | ▾ |
| Interaction between L1 and Ankyrins | 28 | 33 | 0.002 | 0.00114 | 0.0225 | 4 | 4 | 0.000 | ▾ |
| Dissolution of Fibrin Clot | 12 | 14 | 0.001 | 0.00179 | 0.0327 | 19 | 19 | 0.001 | ▴ |
| Defective HLCS causes multiple | 6 | 10 | 0.001 | 0.00148 | 0.0281 | 4 | 4 | 0.000 | ▾ |
| carboxylase deficiency | | | | | | | | | |

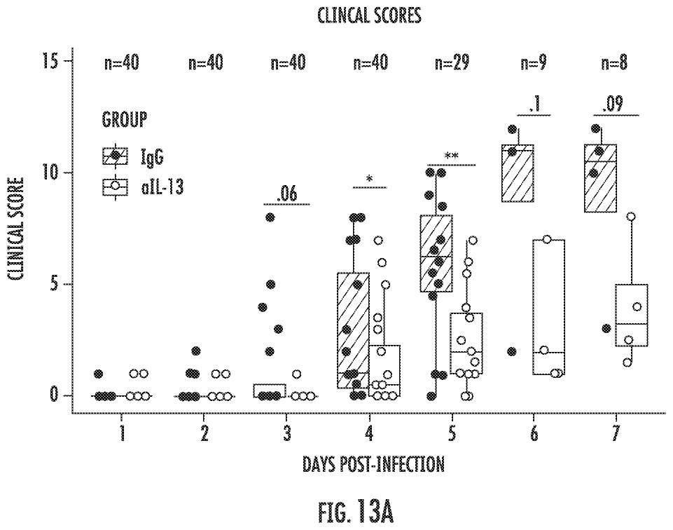

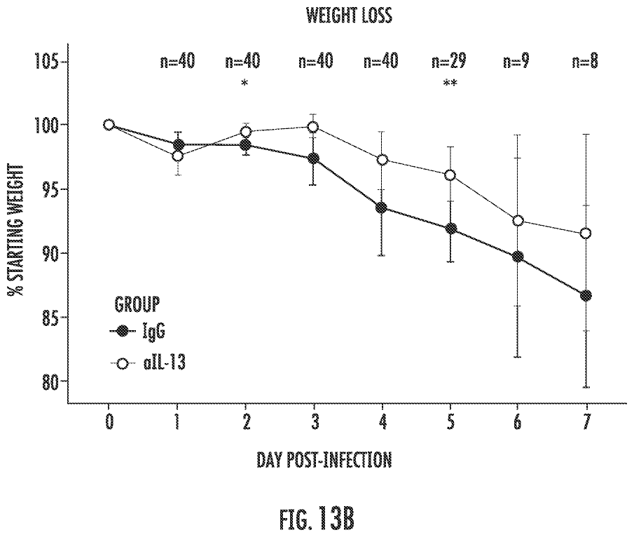

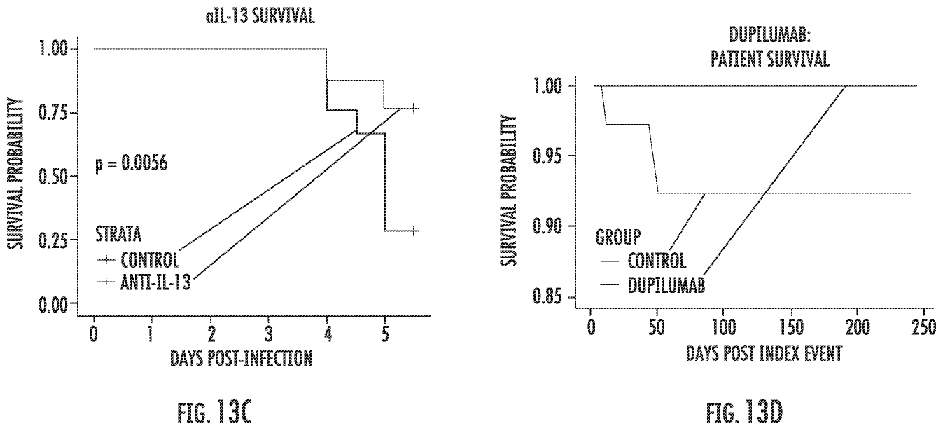

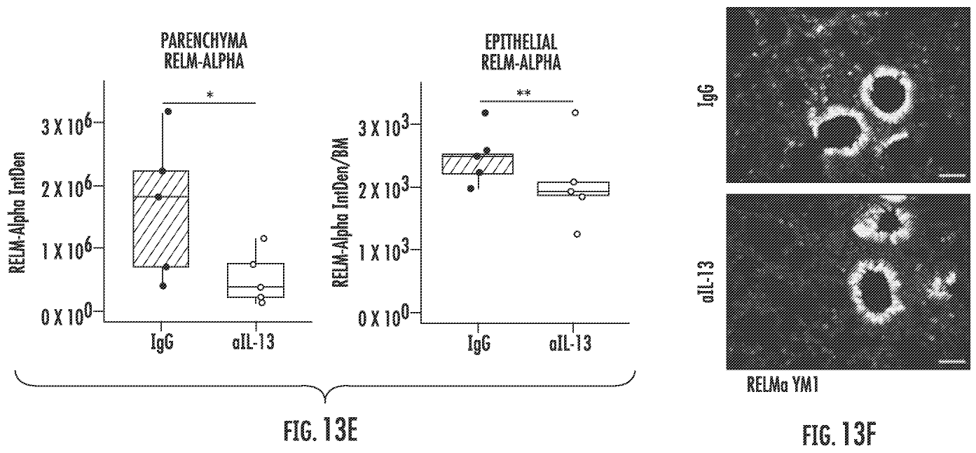

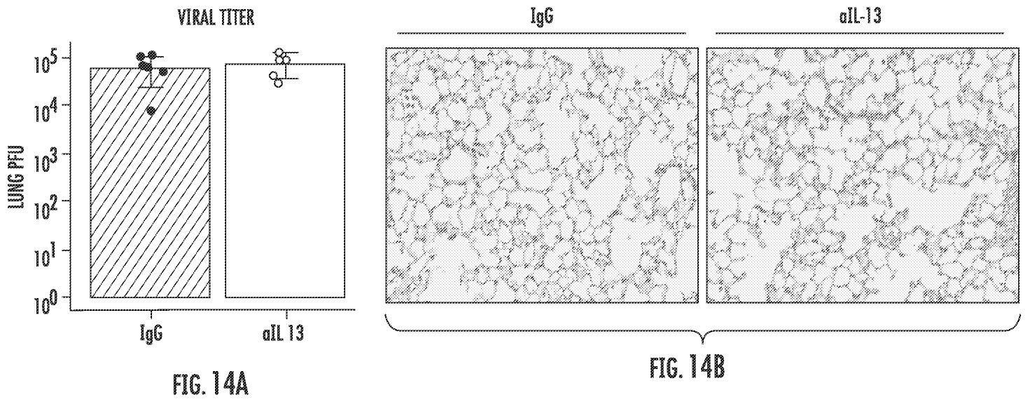

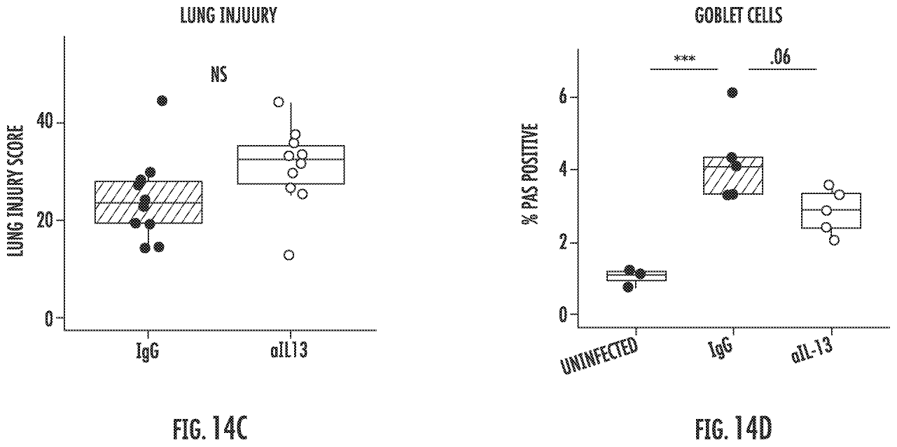

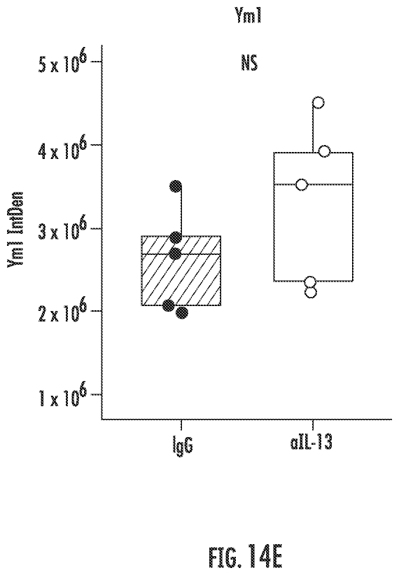

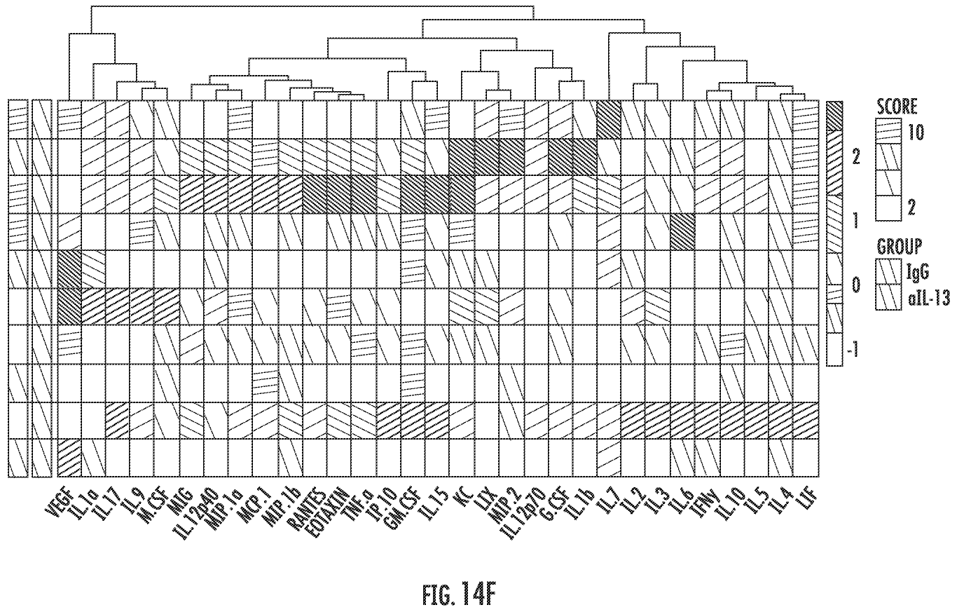

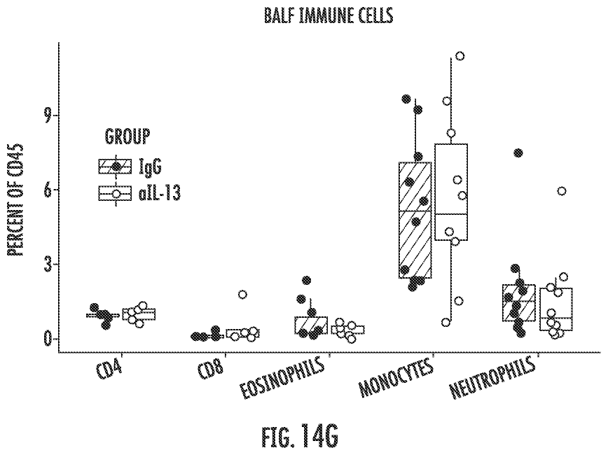

To more directly test whether IL-13 was deleterious following SARS-CoV-2 infection, intraperitoneal (i.p.) injections of anti-IL-13 or an isotype matched control IgG were administered during infection on days 0, 2 and 4 post-infection. Infected mice receiving anti-IL-13 had significantly reduced clinical scores (see Moreau et al., 2020) (see FIG. 13A), weight loss (see FIG. 13B), and mortality (see FIG. 13C) from day three onwards, demonstrating a pathogenic role for IL-13 during disease. Neutralization of IL-13 did not alter viral load in the lungs on day five (see FIG. 14A), suggesting disease amelioration was not due to reduced infectious burden but likely increased pathophysiological responses, including inflammation.

Dupilumab is a monoclonal antibody that blocks IL-13 and IL-4 signaling. It is directed against the IL-4Rα subunit that is shared with the IL-13 receptor (Le Floc'h et al., 2020). Based on the presently disclosed findings, the possibility was considered that patients prescribed Dupilumab for asthma, atopic dermatitis or allergic sinusitis can be protected from severe COVID-19. To test this hypothesis, a retrospective analysis of a large international COVID-19 cohort comprised of 350,004 cases; 81 of which had been prescribed Dupilumab prior to, and independently of their COVID-19 diagnosis was conducted. See Table 5, below. A sub cohort using 1:1 propensity score matching as well as an additional sub cohort for patients with diagnosed asthma, atopic dermatitis or rhinosinusitis, for which Dupilumab is prescribed, were generated. Dupilumab use was associated with a lower risk of ventilation and death in COVID-19. See FIG. 13D and Table 5, below. To assess whether Dupilumab was associated with clinical proxies of inflammation, levels of C-reactive protein (CRP), an acute phase protein that increases during inflammation and correlates with poor outcomes in COVID-19 (Liu et al, 2020), was assessed. CRP levels were reduced in Dupilumab-prescribed patients with COVID-19 (see Table 6, below), suggesting that blocking type 2 immunity can lower overall disease pathology and increase survival rates. As a validation cohort Dupilumab and COVID-19 were also examined in the National COVID Cohort Collaborative (N3C). Out of a total of 785 patients with a record of dupilumab prescription, 31 had a positive COVID-19 test within 61 days after a dupilumab dose, resulting in a test positivity rate of 3.9% (95% CI: 2.8, 5.6). Five matched controls from the N3C for each of the 31 COVID-19 positive, dupilumab prescribed patients were also examined. The 31 patients in who had contracted COVID-19 while on dupilumab also had a lower rate of hospitalization, ventilation, and death compared to matched controls. See Table 7, below.

TABLE 5
Outcomes with and without Dupilumab in the International Cohort.

| | | |
|---|---|---|
| With | Without | Risk Difference |
| Dupilumab | Dupilumab | (95% CI) |

Full Cohort

| | | | |
|---|---|---|---|
| Patients | 81 | 350,004 | |
| Ventilated | 0 | 1,307 | 0.37% (0.35-0.39) |
| Deceased | 0 | 6,942 | 1.98% (1.94-2.03) |

1:1 Propensity Score Matching

| | | | |
|---|---|---|---|
| Patients | 81 | 81 | |
| Deceased | 0 | 10 | 12.3% (5.2-19.5) |

Diagnoses Matching

| | | | |
|---|---|---|---|
| Patients | 65 | 35,564 | |
| Ventilated | 0 | 238 | 0.67% (0.58-0.75) |
| Deceased | 0 | 652 | 1.83% (1.69-1.97) |

TABLE 6
Lab Values and Standard Deviations (SD) for Serum Levels
of C-Reactive Protein (CRP) for COVID-19 Patients.

| | CRP | | Sub-cohort |
|---|---|---|---|
| Group | (mg/l) | SD | Difference |
| Survived | 34.3 | 58.0 | 65.7 |
| Deceased | 100 | 107.0 | |
| Not Ventilated | 39.6 | 65.6 | 35.3 |
| Ventilated | 74.9 | 98.5 | |
| With Dupilumab | 25.3 | 68.3 | 12. |
| Without Dupilumab | 37.6 | 63.8 | |

TABLE 7
N3C Dupilumab COVID Severity Outcomes.

| | | Matched | | |
|---|---|---|---|---|
| | With | Controls | | |
| | Concurrent | without | Odds | |
| | Dupilumab | Dupilumab | Ratio | P-value |
| Patients | 31 | 155 | | |
| Hospitalized | <20 | <20 | 0.64 | 0.57 |
| Ventilated | 0 | <20 | n/a | >0.99 |
| Deceased | 0 | <20 | n/a | >0.99 |

To further understand the mechanism by which IL-13 exacerbated disease, the mouse model was used to investigate whether the reduction in disease severity following IL-13 neutralization corresponded with changes in the lung tissue. Histological parameters of pathology were assessed by H&E staining. Infection with SARS-CoV-2 in this model results in lung damage, however, IL-13 blockade resulted in little change in lung injury (FIGS. 14B and 14C). In contrast, goblet cell metaplasia was subtly, albeit significantly, induced following infection, and this increase was reduced by neutralization of IL-13 (FIG. 14D). During type 2 inflammation in the lung, IL-13 is a significant driver of goblet cell responses (Marone et al., 2019), and the present data provides evidence that IL-13 signaling is active in COVID-19 but less dramatic, histologically, than in other models of type 2 immunity (Borthwick et al., 2016; and Lee et al., 2011).

As further assess how IL-13 neutralization protected from COVID-19, expression of the type 2 associated proteins Ym1 and RELMα was assessed. Fluorescence microscopy of the lung revealed a significant decrease in RELMα following neutralization of IL-13, which was evident in both the parenchyma and within the epithelial cells. See FIGS. 13E and 13F. However, no change in Ym1 following IL-13 neutralization was detected. See FIG. 14E. In addition, no overt changes in cytokine levels or cell composition in the bronchoalveolar lavage fluid (BALF) were observed. See FIGS. 14F and 14G. Accordingly, it was concluded that the mechanism by which IL-13 was promoting more severe COVID-19 was not necessarily through the type 2 pathways typically observed in the lung.

### SUMMARY

The type 2 cytokine, IL-13, is associated with increased COVID-19 severity in patients from multiple patient cohorts, as well as in a mouse model of disease. The IL-13 blocking drug, Dupilumab, in turn, is associated with better outcomes in COVID-19 patients and additionally, neutralization of IL-13 in mice infected with SARS-CoV-2 protects from death. Neutralization of IL-13 also resulted in significant downregulation of Arg1, the gene that encodes for Arginase-1 (Arg1). Arg1 expression is often utilized as a marker for alternatively-activated macrophages (AAMs), which IL-13 is known to promote. Additionally, RELMα can be a marker of AAMs, and in combination with the observation of decreased Arg1 suggests that these macrophages could be downstream of IL-13 signaling during COVID-19. Overall, this work in humans and mice implicates IL-13 as an important driver of severe outcomes during COVID-19.

### REFERENCES

The references listed below as well as all references cited in the specification are incorporated herein by reference to the extent that they supplement, explain, provide a background for or teach methodology, techniques and/or compositions employed herein.

All cited patents and publications referred to in this application are herein expressly incorporated by reference.
Bao et al. “The pathogenicity of SARS-CoV-2 I nhACE2 transgenic mice.” Nature, 583:830-833 (2020).
Bornstein et al. “Practical recommendations for the management of diabetes in patients with COVID-19.” The Lancet Diabetes and Endocrinology, 8(6):546-550 (2020).
Borthwick et al. “Macrophages are critical to the maintenance of IL-13-dependent lung inflammation and fibrosis.” Mucosal Immunol., 9:38-55 (2016).
Chen et al. “Clinical and Immunological Features of Severe and Moderate Coronavirus Disease 2019.” The Journal of Clinical Investigation 130 (5): 2620-2 (2020).
Chiaramonte et al., “Studies of murine schistosomiasis reveal interleukin-13 blockade as a treatment for established and progressive liver fibrosis.” Hepatology, 34, 273-282 (2001).
Del Valle et al. “An inflammatory cytokine signature predicts COVID-19 severity and survival.” Nat. Med., 26:1636-1643 (2020).
Dobin and Gingeras. “Mapping RNA-seq Reads with STAR.” Current Protocols in Bioinformatics, 51(1) (2015). doi:10.1002/0471250953.bil114s51.
Ewels et al. “MultiQC: summarize analysis results for multiple tools and samples in a single report.” Bioinformatics, 32(19):3047-3048 (2016).
Fry et al. “The Many Faces of IL-7: From Lymphopoiesis to Peripheral T Cell Maintenance.” Journal of Immunology, 174 (11): 6571-76 (2005).
Furumoto and Gadina. “The arrival of JAK inhibitors: advancing the treatment of immune and hematologic disorders.” Bio Drugs, 27(5):431-438 (2013).
Griss et al. “ReactomeGSA—Efficient Multi-Omics Comparative Pathway Analysis.” Molecular& Cellular Proteomics. 19(12):2115-2125 (2020).
Guo et al. 2020. “Diabetes Is a Risk Factor for the Progression and Prognosis of COVID-19.” Diabetes/metabolism Research and Reviews, e3319 (2020).
Guttman-Yassky et al. “Efficacy and Safety of Lebrikizumab, a High-Affinity Interleukin 13 Inhibitor, in Adults With Moderate to Severe Atopic Dermatitis: A Phase 2b Randomized Clinical Trial.” JAMA Dennatology, 156(4):411-420 (2020).
Hoffmann et al. “SARS-CoV-2 cell entry depends on ACE2 and TMPRSS2 and is blocked by a clinically proven protease inhibitor.” Cell 181:271-280.e8 (2020).
Huang et al. 2020. “Clinical Features of Patients Infected with 2019 Novel Coronavirus in Wuhan, China.” The Lancet 395 (10223):497-506 (2020).
Junttila. “Tuning the cytokine responses: an update on interleukin (IL)-4 and IL-13 receptor complexes,” Front Immunol., 9:888 (2018).
Le Floc'h et al. “Dual blockade of IL-4 and IL-13 with dupilumab, an IL-4Rα antibody, is required to broadly inhibit type 2 inflammation.” Allergy, 75:1188-1204 (2020).
Lee et al., “Role of Chitin and Chitinase/Chitinase-Like Proteins in Inflammation, Tissue Remodeling, and Injury.” Annu. Rev. Physiol., 73:479-501 (2011).
Leist et al. “A Mouse-Adapted SARS-CoV-2 Induces Acute Lung Injury and Mortality in Standard Laboratory Mice.” Cell, 183: 1070-1085.el2 (2020).
Liao et al. “Single-cell landscape of bronchoalveolar immune cells in patients with COVID-19.” Nat. Med., 26:842-844 (2020).
Liu et al. “Longitudinal Characteristics of Lymphocyte Responses and Cytokine Profiles in the Peripheral Blood of SARS-CoV-2 Infected Patients.” EBioMedicine 55: 102763 (2020).
Love et al. “Moderated estimation of fold change and dispersion for RNA-seq data with DESeq2.” Genome Biol., 15(12):550 (2014).
Lucas et al. “Longitudinal analyses reveal immunological misfiring in severe COVID-19.” Nature, 584:463-469 (2020).
Mangalmurti and Hunter. “Cytokine Storms: Understanding COVID-19.” Immunity, 53:19-25 (2020).
Marone et al. “The Intriguing Role of Interleukin 13 in the Pathophysiology of Asthma.” Front. Pharmacol., 10:1387 (2019).
Mehta et al. “Interleukin-1 Receptor Antagonist (IL-1Ra) Is More Effective in Suppressing Cytokine-Induced Catabolism in Cartilage-Synovium Co-Culture than in Cartilage Monoculture.” Arthritis Research & Therapy 21 (1): 238 (2019).
Merad & Martin. “Pathological inflammation in patients with COVID-19: a key role for monocytes and macrophages.” Nature Reviews Immunology, 20:355-362 (2020).
Mogul et al. “Baricitinib: the second FDA-approved JAK inhibitor for the treatment of rheumatoid arthritis.” Annals of Pharmacotherapy, 53(9):947-953 (2019).
Moreau et al. “Evaluation of K18-hACE2 Mice as a Model of SARS-CoV-2 Infection.” Am. J. Trop. Med. Hyg., 103:1215-1219 (2020).
Muzio et al. “Interleukin-13 Induces the Production of Interleukin-1 Receptor Antagonist (IL-1ra) and the Expression of the mRNA for the Intracellular (keratinocyte) Form of IL-1ra in Human Myelomonocytic Cells.” Blood 83 (7): 1738-43 (1994).
Nienhold et al. “Two distinct immunopathological profiles in autopsy lungs of COVID-19.” Nat. Commun., 11:5086 (2020).
Pedersen & Ho. “SARS-CoV-2: a storm is raging.” J Clin. Invest., 130: 2202-2205 (2020).
Rathnasinghe et al. “Comparison of transgenic and adenovirus hACE2 mouse models for SARS-CoV-2 infection.” Emerg. Microbes Infect., 9:2433-2445 (2020).
Sanderson. “Interleukin-5, Eosinophils, and Disease.” Blood, 79 (12):3101-9 (1992).
Scotton et al. “Transcriptional Profiling Reveals Complex Regulation of the Monocyte IL-1β System by IL-13.” The Journal of Immunology, 174 (2):834-845 (2005).
Shreberk-Hassidim et al. “Janus kinase inhibitors in dermatology: a systematic review.” J. Am. Acad. Dermatol., 76(4):745-753.e19 (2017).
Singh et al. “Diabetes in COVID-19: Prevalence, pathophysiology, prognosis and practical considerations.” Diabetes & Metabolic Syndrome: Clinical Research & Reviews, 14(4):303-310 (2020).
The RECOVERY Collaborative Group. “Dexamethasone in Hospitalized Patients with Covid-19 Preliminary Report.” Published online (Jul. 17, 2020) at NEJM.org doi:10.1056/NEJMoa2021436 (2020).
Tisoncik et al. “Into the eye of the cytokine storm.” Microbiology and Molecular Biology Reviews, 76(1): 16-32 (2012).
Winkler et al. “Neuropilin-1 facilitates SARS-CoV-2 cell entry and infectivity.” Science, 370:856-860 (2020a).
Winkler et al. “SARS-CoV-2 infection of human ACE2-transgenic mice causes severe lung inflammation and impaired function.” Nat. Immunol., 21:1470-1470 (2020b).
Wollenberg et al. “Treatment of atopic dermatitis with tralokinumab, an anti-IL-13 mAb.” J Allergy Clin. Immunol., 143(1):135-141 (2019).
Wu & Smyth. “Camera: a competitive gene set test accounting for inter-gene correlation.” Nucleic Acids Research, 40(17):e133-e133 (2012).
Zaim et al. “COVID-19 and Multiorgan Response.” Current Problems in Cardiology, 45(8):100618 (2020).
Zhou et al. “Clinical Course and Risk Factors for Mortality of Adult Inpatients with COVID-19 in Wuhan, China: A Retrospective Cohort Study.” The Lancet, 395 (10229):1054-1062 (2020).
Zhu et al. “IL-13-Induced Chemokine Responses in the Lung: Role of CCR2 in the Pathogenesis of IL-13-Induced Inflammation and Remodeling.” Journal of Immunology, 168 (6): 2953-62 (2002).

It will be understood that various details of the presently disclosed subject matter may be changed without departing from the scope of the presently disclosed subject matter. Furthermore, the foregoing description is for the purpose of illustration only, and not for the purpose of limitation.

1. A method of treating coronavirus disease 2019 (COVID-19) in a subject in need thereof, the method comprising administering to the subject a composition that comprises a therapeutically effective amount of at least one agent that neutralizes one or more of interleukin-13 (IL-13), interleukin-6 (IL-6) and Janus kinase (JAK).

2. The method of claim 1, wherein the composition comprises a therapeutically effective amount of at least one agent that neutralizes IL-13.

3. The method of claim 2, wherein the at least one agent that neutralizes IL-13 also neutralizes interleukin-4 (IL-4), optionally wherein the at least one agent is an antagonist of IL-4 receptor alpha 1 (IL4Rα1), further optionally wherein the at least one agent is an antibody that binds to IL4Rα1.

4. The method of claim 2, wherein the at least one agent that neutralizes IL-13 is an antibody, optionally wherein the antibody is a monoclonal antibody selected from the group consisting of dupilumab, lebrikizumab, and tralokinumab, further optionally wherein the monoclonal antibody is dupilumab.

5. The method of claim 1, wherein the composition comprises a therapeutically effective amount of at least one agent that neutralizes IL-6, optionally wherein the at least one agent is an antibody, further optionally wherein the antibody is a monoclonal antibody selected from the group consisting of tocilizumab and sarilumab.

6. The method of claim 1, wherein the composition comprises a therapeutically effective amount of at least one agent that neutralizes JAK, optionally wherein the at least one agent is a small molecule JAK inhibitor, further optionally wherein the small molecule JAK inhibitor is selected from the group consisting of baricitinib and tofacitinib.

7. The method of claim 1, wherein treating COVID-19 reduces pulmonary inflammation.

8. The method of claim 1, wherein treating COVID-19 reduces the risk of one or more of ventilation dependence, hospitalization, and death.

9. A method of predicting increased risk for mechanical ventilation in a subject having coronavirus disease 2019 (COVID-19), the method comprising:
(a) measuring a plasma concentration of one or more cell signaling protein in the subject, wherein the one or more cell signaling protein comprises at least one selected from the group consisting of interleukin-13 (IL-13), interleukin-7 (IL-7), and basic fibroblast growth factor (FGF-basic);
(b) comparing the plasma concentration measured in the subject with a reference concentration of the one or more cell signaling protein;
(c) predicting that the subject has an increased risk for mechanical ventilation when the plasma concentration measured in the subject is higher than the reference concentration or that the subject does not have an increased risk for mechanical ventilation when the plasma concentration measured in the subject is not higher than the reference concentration; and
(d) administering to the subject a composition that comprises a therapeutically effective amount of at least one agent that neutralizes one or more of interleukin-13 (IL-13), interleukin-6 (IL-6) and Janus kinase (JAK) if the subject is predicted to have an increased risk of mechanical ventilation.

10. The method of claim 9, wherein step (a) comprises measuring a plasma concentration of IL-13.

11. The method of claim 10, wherein step (a) further comprises measuring a plasma concentration of IL-6.

12. The method of claim 11, wherein step (a) further comprises measuring a plasma concentration of interleukin-8 (IL-8) and macrophage inflammatory protein-1 beta (MIP-1β).


SEQUENCE LISTING (19 sequences)

SEQ ID NO: 1 - protein, 146 aa, Homo sapiens
MHPLLNPLLLALGLMALLLTTVIALTCLGGFASPGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGQFN

SEQ ID NO: 2 - protein, 81 aa, Homo sapiens
MVWSINLTAGMYCAALESLINVSGCSAIEKTQRMLSGFCPHKVSAGQFSSLHVRDTKIEVAQFVKDLLLHLKKLFREGQFN

SEQ ID NO: 3 - protein, 427 aa, Homo sapiens
MEWPARLCGLWALLLCAGGGGGGGGAAPTETQPPVTNLSVSVENLCTVIWTWNPPEGASSNCSLWYFSHFGDKQDKKIAPETRRSIEVPLNERICLQVGSQCSTNESEKPSILVEKCISPPEGDPESAVTELQCIWHNLSYMKCSWLPGRNTSPDTNYTLYYWHRSLEKIHQCENIFREGQYFGCSFDLTKVKDSSFEQHSVQIMVKDNAGKIKPSFNIVPLTSRVKPDPPHIKNLSFHNDDLYVQWENPQNFISRCLFYEVEVNNSQTETHNVFYVQEAKCENPEFERNVENTSCFMVPGVLPDTLNTVRIRVKTNKLCYEDDKLWSNWSQEMSIGKKRNSTLYITMLLIVPVIVAGAIIVLLLYLKRLKIIIFPPIPDPGKIFKEMFGDQNDDTLHWKKYDIYEKQTKEETDSVVLIENLKKASQ

SEQ ID NO: 4 - protein, 825 aa, Homo sapiens
MGWLCSGLLFPVSCLVLLQVASSGNMKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQHLLLGVSVSCIVILAVCLLCYVSITKIKKEWWDQIPNPARSRLVAIIIQDAQGSQWEKRSRGQEPAKCPHWKNCLTKLLPCFLEHNMKRDEDPHKAAKEMPFQGSGKSAWCPVEISKTVLWPESISVVRCVELFEAPVECEEEEEVEEEKGSFCASPESSRDDFQEGREGIVARLTESLFLDLLGEENGGFCQQDMGESCLLPPSGSTSAHMPWDEFPSAGPKEAPPWGKEQPLHLEPSPPASPTQSPDNLTCTETPLVIAGNPAYRSFSNSLSQSPCPRELGPDPLLARHLEEVEPEMPCVPQLSEPTTVPQPEPETWEQILRRNVLQHGAAAAPVSAPTSGYQEFVHAVEQGGTQASAVVGLGPPGEAGYKAFSSLLASSAVSPEKCGFGASSGEEGYKPFQDLIPGCPGDPAPVPVPLFTFGLDREPPRSPQSSHLPSSSPEHLGLEPGEKVEDMPKPPLPQEQATDPLVDSLGSGIVYSALTCHLCGHLKQCHGQEDGGQTPVMASPCCGCCCGDRSSPPTTPLRAPDPSPGGVPLEASLCPASLAPSGISEKSKSSSSFHPAPGNAQSSSQTPKIVNFVSVGPTYMRVS

SEQ ID NO: 5 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 6 - protein, 136 aa, Homo sapiens
MCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 7 - protein, 189 aa, Homo sapiens
MNSFSTIPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 8 - DNA, 1283 bp, Homo sapiens
aagccacccagcctatgcatccgctcctcaatcctctcctgttggcactgggcctcatggcgcttttgttgaccacggtcattgctctcacttgccttggcggctttgcctccccaggccctgtgcctccctctacagccctcagggagctcattgaggagctggtcaacatcacccagaaccagaaggctccgctctgcaatggcagcatggtatggagcatcaacctgacagctggcatgtactgtgcagccctggaatccctgatcaacgtgtcaggctgcagtgccatcgagaagacccagaggatgctgagcggattctgcccgcacaaggtctcagctgggcagttttccagcttgcatgtccgagacaccaaaatcgaggtggcccagtttgtaaaggacctgctcttacatttaaagaaactttttcgcgagggacagttcaactgaaacttcgaaagcatcattatttgcagagacaggacctgactattgaagttgcagattcatttttctttctgatgtcaaaaatgtcttgggtaggcgggaaggagggttagggaggggtaaaattccttagcttagacctcagcctgtgctgcccgtcttcagcctagccgacctcagccttccccttgcccagggctcagcctggtgggcctcctctgtccagggccctgagctcggtggacccagggatgacatgtccctacacccctcccctgccctagagcacactgtagcattacagtgggtgccccccttgccagacatgtggtgggacagggacccacttcacacacaggcaactgaggcagacagcagctcaggcacacttcttcttggtcttatttattattgtgtgttatttaaatgagtgtgtttgtcaccgttggggattggggaagactgtggctgctagcacttggagccaagggttcagagactcagggccccagcactaaagcagtggacaccaggagtccctggtaataagtactgtgtacagaattctgctacctcactggggtcctggggcctcggagcctcatccgaggcagggtcaggagaggggcagaacagccgctcctgtctgccagccagcagccagctctcagccaacgagtaatttattgtttttccttgtatttaaatattaaatatgttagcaaagagttaatatatagaagggtaccttgaacactgggggaggggacattgaacaagttgtttcattgactatcaaactgaagccagaaataaagttggtgacagata

SEQ ID NO: 9 - DNA, 1275 bp, Homo sapiens
acgcgcgggggcgcccctgcccaccgctcccggcagggcttttggtggccatgggggataaggggcgttgactcacccgggcggggctccgggagttgcacagaccaaggccctgtgcctccctctacagccctcagggagctcattgaggagctggtcaacatcacccagaaccagaaggctccgctctgcaatggcagcatggtatggagcatcaacctgacagctggcatgtactgtgcagccctggaatccctgatcaacgtgtcaggctgcagtgccatcgagaagacccagaggatgctgagcggattctgcccgcacaaggtctcagctgggcagttttccagcttgcatgtccgagacaccaaaatcgaggtggcccagtttgtaaaggacctgctcttacatttaaagaaactttttcgcgagggacagttcaactgaaacttcgaaagcatcattatttgcagagacaggacctgactattgaagttgcagattcatttttctttctgatgtcaaaaatgtcttgggtaggcgggaaggagggttagggaggggtaaaattccttagcttagacctcagcctgtgctgcccgtcttcagcctagccgacctcagccttccccttgcccagggctcagcctggtgggcctcctctgtccagggccctgagctcggtggacccagggatgacatgtccctacacccctcccctgccctagagcacactgtagcattacagtgggtgccccccttgccagacatgtggtgggacagggacccacttcacacacaggcaactgaggcagacagcagctcaggcacacttcttcttggtcttatttattattgtgtgttatttaaatgagtgtgtttgtcaccgttggggattggggaagactgtggctgctagcacttggagccaagggttcagagactcagggccccagcactaaagcagtggacaccaggagtccctggtaataagtactgtgtacagaattctgctacctcactggggtcctggggcctcggagcctcatccgaggcagggtcaggagaggggcagaacagccgctcctgtctgccagccagcagccagctctcagccaacgagtaatttattgtttttccttgtatttaaatattaaatatgttagcaaagagttaatatatagaagggtaccttgaacactgggggaggggacattgaacaagttgtttcattgactatcaaactgaagccagaaataaagttggtgacagata

SEQ ID NO: 10 - DNA, 1454 bp, Homo sapiens
acgcgcgggggcgcccctgcccaccgctcccggcagggcttttggtggccatgggggataaggggcgttgactcacccgggcggggctccgggagttgcacagaccaaggcaaatgccgtggcctctgctgtgggggtttctggaggacttctaggaaaacgagggaagagcaggaaaaggcgacatggctgcaggggccaagcccaggagccgccctccacagcactcattctgcagaagggaaatttgaggcccccagacggcaggggttgatcctgcagagactggccctgtgcctccctctacagccctcagggagctcattgaggagctggtcaacatcacccagaaccagaaggctccgctctgcaatggcagcatggtatggagcatcaacctgacagctggcatgtactgtgcagccctggaatccctgatcaacgtgtcaggctgcagtgccatcgagaagacccagaggatgctgagcggattctgcccgcacaaggtctcagctgggcagttttccagcttgcatgtccgagacaccaaaatcgaggtggcccagtttgtaaaggacctgctcttacatttaaagaaactttttcgcgagggacagttcaactgaaacttcgaaagcatcattatttgcagagacaggacctgactattgaagttgcagattcatttttctttctgatgtcaaaaatgtcttgggtaggcgggaaggagggttagggaggggtaaaattccttagcttagacctcagcctgtgctgcccgtcttcagcctagccgacctcagccttccccttgcccagggctcagcctggtgggcctcctctgtccagggccctgagctcggtggacccagggatgacatgtccctacacccctcccctgccctagagcacactgtagcattacagtgggtgccccccttgccagacatgtggtgggacagggacccacttcacacacaggcaactgaggcagacagcagctcaggcacacttcttcttggtcttatttattattgtgtgttatttaaatgagtgtgtttgtcaccgttggggattggggaagactgtggctgctagcacttggagccaagggttcagagactcagggccccagcactaaagcagtggacaccaggagtccctggtaataagtactgtgtacagaattctgctacctcactggggtcctggggcctcggagcctcatccgaggcagggtcaggagaggggcagaacagccgctcctgtctgccagccagcagccagctctcagccaacgagtaatttattgtttttccttgtatttaaatattaaatatgttagcaaagagttaatatatagaagggtaccttgaacactgggggaggggacattgaacaagttgtttcattgactatcaaactgaagccagaaataaagttggtgacagata

SEQ ID NO: 11 - DNA, 1383 bp, Homo sapiens
acgcgcgggggcgcccctgcccaccgctcccggcagggcttttggtggccatgggggataaggggcgttgactcacccgggcggggctccgggagttgcacagaccaaggcaaatgccgtggcctctgctgtgggggtttctggaggacttctaggaaaacgagggaagagcaggaaaaggcgacatggctgcaggggccaagcccaggagccgccctccacagcactcattctgcagaagggaaatttgaggcccccagacggcaggggttgatcctgcagagactggctccgctctgcaatggcagcatggtatggagcatcaacctgacagctggcatgtactgtgcagccctggaatccctgatcaacgtgtcaggctgcagtgccatcgagaagacccagaggatgctgagcggattctgcccgcacaaggtctcagctgggcagttttccagcttgcatgtccgagacaccaaaatcgaggtggcccagtttgtaaaggacctgctcttacatttaaagaaactttttcgcgagggacagttcaactgaaacttcgaaagcatcattatttgcagagacaggacctgactattgaagttgcagattcatttttctttctgatgtcaaaaatgtcttgggtaggcgggaaggagggttagggaggggtaaaattccttagcttagacctcagcctgtgctgcccgtcttcagcctagccgacctcagccttccccttgcccagggctcagcctggtgggcctcctctgtccagggccctgagctcggtggacccagggatgacatgtccctacacccctcccctgccctagagcacactgtagcattacagtgggtgccccccttgccagacatgtggtgggacagggacccacttcacacacaggcaactgaggcagacagcagctcaggcacacttcttcttggtcttatttattattgtgtgttatttaaatgagtgtgtttgtcaccgttggggattggggaagactgtggctgctagcacttggagccaagggttcagagactcagggccccagcactaaagcagtggacaccaggagtccctggtaataagtactgtgtacagaattctgctacctcactggggtcctggggcctcggagcctcatccgaggcagggtcaggagaggggcagaacagccgctcctgtctgccagccagcagccagctctcagccaacgagtaatttattgtttttccttgtatttaaatattaaatatgttagcaaagagttaatatatagaagggtaccttgaacactgggggaggggacattgaacaagttgtttcattgactatcaaactgaagccagaaataaagttggtgacagata

SEQ ID NO: 12 - DNA, 3996 bp, Homo sapiens
ccagcccggccgggctccgaggcgagaggctgcatggagtggccggcgcggctctgcgggctgtgggcgctgctgctctgcgccggcggcgggggcgggggcgggggcgccgcgcctacggaaactcagccacctgtgacaaatttgagtgtctctgttgaaaacctctgcacagtaatatggacatggaatccacccgagggagccagctcaaattgtagtctatggtattttagtcattttggcgacaaacaagataagaaaatagctccggaaactcgtcgttcaatagaagtacccctgaatgagaggatttgtctgcaagtggggtcccagtgtagcaccaatgagagtgagaagcctagcattttggttgaaaaatgcatctcacccccagaaggtgatcctgagtctgctgtgactgagcttcaatgcatttggcacaacctgagctacatgaagtgttcttggctccctggaaggaataccagtcccgacactaactatactctctactattggcacagaagcctggaaaaaattcatcaatgtgaaaacatctttagagaaggccaatactttggttgttcctttgatctgaccaaagtgaaggattccagttttgaacaacacagtgtccaaataatggtcaaggataatgcaggaaaaattaaaccatccttcaatatagtgcctttaacttcccgtgtgaaacctgatcctccacatattaaaaacctctccttccacaatgatgacctatatgtgcaatgggagaatccacagaattttattagcagatgcctattttatgaagtagaagtcaataacagccaaactgagacacataatgttttctacgtccaagaggctaaatgtgagaatccagaatttgagagaaatgtggagaatacatcttgtttcatggtccctggtgttcttcctgatactttgaacacagtcagaataagagtcaaaacaaataagttatgctatgaggatgacaaactctggagtaattggagccaagaaatgagtataggtaagaagcgcaattccacactctacataaccatgttactcattgttccagtcatcgtcgcaggtgcaatcatagtactcctgctttacctaaaaaggctcaagattattatattccctccaattcctgatcctggcaagatttttaaagaaatgtttggagaccagaatgatgatactctgcactggaagaagtacgacatctatgagaagcaaaccaaggaggaaaccgactctgtagtgctgatagaaaacctgaagaaagcctctcagtgatggagataatttatttttaccttcactgtgaccttgagaagattcttcccattctccatttgttatctgggaacttattaaatggaaactgaaactactgcaccatttaaaaacaggcagctcataagagccacaggtctttatgttgagtcgcgcaccgaaaaactaaaaataatgggcgctttggagaagagtgtggagtcattctcattgaattataaaagccagcaggcttcaaactaggggacaaagcaaaaagtgatgatagtggtggagttaatcttatcaagagttgtgacaacttcctgagggatctatacttgctttgtgttctttgtgtcaacatgaacaaattttatttgtaggggaactcatttggggtgcaaatgctaatgtcaaacttgagtcacaaagaacatgtagaaaacaaaatggataaaatctgatatgtattgtttgggatcctattgaaccatgtttgtggctattaaaactcttttaacagtctgggctgggtccggtggctcacgcctgtaatcccagcaatttgggagtccgaggcgggcggatcactcgaggtcaggagttccagaccagcctgaccaaaatggtgaaacctcctctctactaaaactacaaaaattaactgggtgtggtggcgcgtgcctgtaatcccagctactcgggaagctgaggcaggtgaattgtttgaacctgggaggtggaggttgcagtgagcagagatcacaccactgcactctagcctgggtgacagagcaagactctgtctaaaaaacaaaacaaaacaaaacaaaacaaaaaaacctcttaatattctggagtcatcattcccttcgacagcattttcctctgctttgaaagccccagaaatcagtgttggccatgatgacaactacagaaaaaccagaggcagcttctttgccaagacctttcaaagccattttaggctgttaggggcagtggaggtagaatgactccttgggtattagagtttcaaccatgaagtctctaacaatgtattttcttcacctctgctactcaagtagcatttactgtgtctttggtttgtgctaggcccccgggtgtgaagcacagaccccttccaggggtttacagtctatttgagactcctcagttcttgccacttttttttttaatctccaccagtcatttttcagaccttttaactcctcaattccaacactgatttccccttttgcattctccctccttcccttccttgtagccttttgactttcattggaaattaggatgtaaatctgctcaggagacctggaggagcagaggataattagcatctcaggttaagtgtgagtaatctgagaaacaatgactaattcttgcatattttgtaacttccatgtgagggttttcagcattgatatttgtgcattttctaaacagagatgaggtggtatcttcacgtagaacattggtattcgcttgagaaaaaaagaatagttgaacctatttctctttctttacaagatgggtccaggattcctcttttctctgccataaatgattaattaaatagcttttgtgtcttacattggtagccagccagccaaggctctgtttatgcttttggggggcatatattgggttccattctcacctatccacacaacatatccgtatatatcccctctactcttacttcccccaaatttaaagaagtatgggaaatgagaggcatttcccccaccccatttctctcctcacacacagactcatattactggtaggaacttgagaactttatttccaagttgttcaaacatttaccaatcatattaatacaatgatgctatttgcaattcctgctcctaggggaggggagataagaaaccctcactctctacaggtttgggtacaagtggcaacctgcttccatggccgtgtagaagcatggtgccctggcttctctgaggaagctggggttcatgacaatggcagatgtaaagttattcttgaagtcagattgaggctgggagacagccgtagtagatgttctactttgttctgctgttctctagaaagaatatttggttttcctgtataggaatgagattaattcctttccaggtattttataattctgggaagcaaaacccatgcctccccctagccatttttactgttatcctatttagatggccatgaagaggatgctgtgaaattcccaacaaacattgatgctgacagtcatgcagtctgggagtggggaagtgatcttttgttcccatcctcttcttttagcagtaaaatagctgagggaaaagggagggaaaaggaagttatgggaatacctgtggtggttgtgatccctaggtcttgggagctcttggaggtgtctgtatcagtggatttcccatcccctgtgggaaattagtaggctcatttactgttttaggtctagcctatgtggattttttcctaacatacctaagcaaacccagtgtcaggatggtaattcttattctttcgttcagttaagtttttcccttcatctgggcactgaagggatatgtgaaacaatgttaacatttttggtagtcttcaaccagggattgtttctgtttaacttcttataggaaagcttgagtaaaataaatattgtctttttgtatgtca

SEQ ID NO: 13 - DNA, 3624 bp, Homo sapiens
acttcccgcttgggcgcccggacggcgaatggagcaggggcgcgcagataattaaagatttacacacagctggaagaaatcatagagaagccgggcgtggtggctcatgcctataatcccagcacttttggaggctgaggcgggcagatcacttgagatcaggagttcgagaccagcctggtgccttggcatctcccaatggggtggctttgctctgggctcctgttccctgtgagctgcctggtcctgctgcaggtggcaagctctgggaacatgaaggtcttgcaggagcccacctgcgtctccgactacatgagcatctctacttgcgagtggaagatgaatggtcccaccaattgcagcaccgagctccgcctgttgtaccagctggtttttctgctctccgaagcccacacgtgtatccctgagaacaacggaggcgcggggtgcgtgtgccacctgctcatggatgacgtggtcagtgcggataactatacactggacctgtgggctgggcagcagctgctgtggaagggctccttcaagcccagcgagcatgtgaaacccagggccccaggaaacctgacagttcacaccaatgtctccgacactctgctgctgacctggagcaacccgtatccccctgacaattacctgtataatcatctcacctatgcagtcaacatttggagtgaaaacgacccggcagatttcagaatctataacgtgacctacctagaaccctccctccgcatcgcagccagcaccctgaagtctgggatttcctacagggcacgggtgagggcctgggctcagtgctataacaccacctggagtgagtggagccccagcaccaagtggcacaactcctacagggagcccttcgagcagcacctcctgctgggcgtcagcgtttcctgcattgtcatcctggccgtctgcctgttgtgctatgtcagcatcaccaagattaagaaagaatggtgggatcagattcccaacccagcccgcagccgcctcgtggctataataatccaggatgctcaggggtcacagtgggagaagcggtcccgaggccaggaaccagccaagtgcccacactggaagaattgtcttaccaagctcttgccctgttttctggagcacaacatgaaaagggatgaagatcctcacaaggctgccaaagagatgcctttccagggctctggaaaatcagcatggtgcccagtggagatcagcaagacagtcctctggccagagagcatcagcgtggtgcgatgtgtggagttgtttgaggccccggtggagtgtgaggaggaggaggaggtagaggaagaaaaagggagcttctgtgcatcgcctgagagcagcagggatgacttccaggagggaagggagggcattgtggcccggctaacagagagcctgttcctggacctgctcggagaggagaatgggggcttttgccagcaggacatgggggagtcatgccttcttccaccttcgggaagtacgagtgctcacatgccctgggatgagttcccaagtgcagggcccaaggaggcacctccctggggcaaggagcagcctctccacctggagccaagtcctcctgccagcccgacccagagtccagacaacctgacttgcacagagacgcccctcgtcatcgcaggcaaccctgcttaccgcagcttcagcaactccctgagccagtcaccgtgtcccagagagctgggtccagacccactgctggccagacacctggaggaagtagaacccgagatgccctgtgtcccccagctctctgagccaaccactgtgccccaacctgagccagaaacctgggagcagatcctccgccgaaatgtcctccagcatggggcagctgcagcccccgtctcggcccccaccagtggctatcaggagtttgtacatgcggtggagcagggtggcacccaggccagtgcggtggtgggcttgggtcccccaggagaggctggttacaaggccttctcaagcctgcttgccagcagtgctgtgtccccagagaaatgtgggtttggggctagcagtggggaagaggggtataagcctttccaagacctcattcctggctgccctggggaccctgccccagtccctgtccccttgttcacctttggactggacagggagccacctcgcagtccgcagagctcacatctcccaagcagctccccagagcacctgggtctggagccgggggaaaaggtagaggacatgccaaagcccccacttccccaggagcaggccacagacccccttgtggacagcctgggcagtggcattgtctactcagcccttacctgccacctgtgcggccacctgaaacagtgtcatggccaggaggatggtggccagacccctgtcatggccagtccttgctgtggctgctgctgtggagacaggtcctcgccccctacaacccccctgagggccccagacccctctccaggtggggttccactggaggccagtctgtgtccggcctccctggcaccctcgggcatctcagagaagagtaaatcctcatcatccttccatcctgcccctggcaatgctcagagctcaagccagacccccaaaatcgtgaactttgtctccgtgggacccacatacatgagggtctcttaggtgcatgtcctcttgttgctgagtctgcagatgaggactagggcttatccatgcctgggaaatgccacctcctggaaggcagccaggctggcagatttccaaaagacttgaagaaccatggtatgaaggtgattggccccactgacgttggcctaacactgggctgcagagactggaccccgcccagcattgggctgggctcgccacatcccatgagagtagagggcactgggtcgccgtgccccacggcaggcccctgcaggaaaactgaggcccttgggcacctcgacttgtgaacgagttgttggctgctccctccacagcttctgcagcagactgtccctgttgtaactgcccaaggcatgttttgcccaccagatcatggcccacgtggaggcccacctgcctctgtctcactgaactagaagccgagcctagaaactaacacagccatcaagggaatgacttgggcggccttgggaaatcgatgagaaattgaacttcagggagggtggtcattgcctagaggtgctcattcatttaacagagcttccttaggttgatgctggaggcagaatcccggctgtcaaggggtgttcagttaaggggagcaacagaggacatgaaaaattgctatgactaaagcagggacaatttgctgccaaacacccatgcccagctgtatggctgggggctcctcgtatgcatggaacccccagaataaatatgctcagccaccctgtgggccgggcaatccagacagcaggcataaggcaccagttaccctgcatgttggcccagacctcaggtgctagggaaggcgggaaccttgggttgagtaatgctcgtctgtgtgttttagtttcatcacctgttatctgtgtttgctgaggagagtggaacagaaggggtggagttttgtataaataaagtttctttgtctcttta

SEQ ID NO: 14 - DNA, 3491 bp, Homo sapiens
acttcccgcttgggcgcccggacggcgaatggagcaggggcgcgcaggtgccttggcatctcccaatggggtggctttgctctgggctcctgttccctgtgagctgcctggtcctgctgcaggtggcaagctctgggaacatgaaggtcttgcaggagcccacctgcgtctccgactacatgagcatctctacttgcgagtggaagatgaatggtcccaccaattgcagcaccgagctccgcctgttgtaccagctggtttttctgctctccgaagcccacacgtgtatccctgagaacaacggaggcgcggggtgcgtgtgccacctgctcatggatgacgtggtcagtgcggataactatacactggacctgtgggctgggcagcagctgctgtggaagggctccttcaagcccagcgagcatgtgaaacccagggccccaggaaacctgacagttcacaccaatgtctccgacactctgctgctgacctggagcaacccgtatccccctgacaattacctgtataatcatctcacctatgcagtcaacatttggagtgaaaacgacccggcagatttcagaatctataacgtgacctacctagaaccctccctccgcatcgcagccagcaccctgaagtctgggatttcctacagggcacgggtgagggcctgggctcagtgctataacaccacctggagtgagtggagccccagcaccaagtggcacaactcctacagggagcccttcgagcagcacctcctgctgggcgtcagcgtttcctgcattgtcatcctggccgtctgcctgttgtgctatgtcagcatcaccaagattaagaaagaatggtgggatcagattcccaacccagcccgcagccgcctcgtggctataataatccaggatgctcaggggtcacagtgggagaagcggtcccgaggccaggaaccagccaagtgcccacactggaagaattgtcttaccaagctcttgccctgttttctggagcacaacatgaaaagggatgaagatcctcacaaggctgccaaagagatgcctttccagggctctggaaaatcagcatggtgcccagtggagatcagcaagacagtcctctggccagagagcatcagcgtggtgcgatgtgtggagttgtttgaggccccggtggagtgtgaggaggaggaggaggtagaggaagaaaaagggagcttctgtgcatcgcctgagagcagcagggatgacttccaggagggaagggagggcattgtggcccggctaacagagagcctgttcctggacctgctcggagaggagaatgggggcttttgccagcaggacatgggggagtcatgccttcttccaccttcgggaagtacgagtgctcacatgccctgggatgagttcccaagtgcagggcccaaggaggcacctccctggggcaaggagcagcctctccacctggagccaagtcctcctgccagcccgacccagagtccagacaacctgacttgcacagagacgcccctcgtcatcgcaggcaaccctgcttaccgcagcttcagcaactccctgagccagtcaccgtgtcccagagagctgggtccagacccactgctggccagacacctggaggaagtagaacccgagatgccctgtgtcccccagctctctgagccaaccactgtgccccaacctgagccagaaacctgggagcagatcctccgccgaaatgtcctccagcatggggcagctgcagcccccgtctcggcccccaccagtggctatcaggagtttgtacatgcggtggagcagggtggcacccaggccagtgcggtggtgggcttgggtcccccaggagaggctggttacaaggccttctcaagcctgcttgccagcagtgctgtgtccccagagaaatgtgggtttggggctagcagtggggaagaggggtataagcctttccaagacctcattcctggctgccctggggaccctgccccagtccctgtccccttgttcacctttggactggacagggagccacctcgcagtccgcagagctcacatctcccaagcagctccccagagcacctgggtctggagccgggggaaaaggtagaggacatgccaaagcccccacttccccaggagcaggccacagacccccttgtggacagcctgggcagtggcattgtctactcagcccttacctgccacctgtgcggccacctgaaacagtgtcatggccaggaggatggtggccagacccctgtcatggccagtccttgctgtggctgctgctgtggagacaggtcctcgccccctacaacccccctgagggccccagacccctctccaggtggggttccactggaggccagtctgtgtccggcctccctggcaccctcgggcatctcagagaagagtaaatcctcatcatccttccatcctgcccctggcaatgctcagagctcaagccagacccccaaaatcgtgaactttgtctccgtgggacccacatacatgagggtctcttaggtgcatgtcctcttgttgctgagtctgcagatgaggactagggcttatccatgcctgggaaatgccacctcctggaaggcagccaggctggcagatttccaaaagacttgaagaaccatggtatgaaggtgattggccccactgacgttggcctaacactgggctgcagagactggaccccgcccagcattgggctgggctcgccacatcccatgagagtagagggcactgggtcgccgtgccccacggcaggcccctgcaggaaaactgaggcccttgggcacctcgacttgtgaacgagttgttggctgctccctccacagcttctgcagcagactgtccctgttgtaactgcccaaggcatgttttgcccaccagatcatggcccacgtggaggcccacctgcctctgtctcactgaactagaagccgagcctagaaactaacacagccatcaagggaatgacttgggcggccttgggaaatcgatgagaaattgaacttcagggagggtggtcattgcctagaggtgctcattcatttaacagagcttccttaggttgatgctggaggcagaatcccggctgtcaaggggtgttcagttaaggggagcaacagaggacatgaaaaattgctatgactaaagcagggacaatttgctgccaaacacccatgcccagctgtatggctgggggctcctcgtatgcatggaacccccagaataaatatgctcagccaccctgtgggccgggcaatccagacagcaggcataaggcaccagttaccctgcatgttggcccagacctcaggtgctagggaaggcgggaaccttgggttgagtaatgctcgtctgtgtgttttagtttcatcacctgttatctgtgtttgctgaggagagtggaacagaaggggtggagttttgtataaataaagtttctttgtctcttta

SEQ ID NO: 15 - DNA, 3735 bp, Homo sapiens
acttcccgcttgggcgcccggacggcgaatggagcaggggcgcgcaggtgccttggcatctcccaatggggtggctttgctctgggctcctgttccctgtgagctgcctggtcctgctgcaggtggcaagctctggactcttcaggatgccgtgtggagaaaggaagagggtggaagccaggaggtctggagggaggtctggagtggaggagatgagaggctccggatccctctgggaggtagatttgaggacagattggaattgaggtgaaagacagagaaagagaagtggccaggatgactccaagatttctgacctaaactactgggaaggacgcggttgtcatttctgaaatgcagaaggatgccagaagagaagggaacatgaaggtcttgcaggagcccacctgcgtctccgactacatgagcatctctacttgcgagtggaagatgaatggtcccaccaattgcagcaccgagctccgcctgttgtaccagctggtttttctgctctccgaagcccacacgtgtatccctgagaacaacggaggcgcggggtgcgtgtgccacctgctcatggatgacgtggtcagtgcggataactatacactggacctgtgggctgggcagcagctgctgtggaagggctccttcaagcccagcgagcatgtgaaacccagggccccaggaaacctgacagttcacaccaatgtctccgacactctgctgctgacctggagcaacccgtatccccctgacaattacctgtataatcatctcacctatgcagtcaacatttggagtgaaaacgacccggcagatttcagaatctataacgtgacctacctagaaccctccctccgcatcgcagccagcaccctgaagtctgggatttcctacagggcacgggtgagggcctgggctcagtgctataacaccacctggagtgagtggagccccagcaccaagtggcacaactcctacagggagcccttcgagcagcacctcctgctgggcgtcagcgtttcctgcattgtcatcctggccgtctgcctgttgtgctatgtcagcatcaccaagattaagaaagaatggtgggatcagattcccaacccagcccgcagccgcctcgtggctataataatccaggatgctcaggggtcacagtgggagaagcggtcccgaggccaggaaccagccaagtgcccacactggaagaattgtcttaccaagctcttgccctgttttctggagcacaacatgaaaagggatgaagatcctcacaaggctgccaaagagatgcctttccagggctctggaaaatcagcatggtgcccagtggagatcagcaagacagtcctctggccagagagcatcagcgtggtgcgatgtgtggagttgtttgaggccccggtggagtgtgaggaggaggaggaggtagaggaagaaaaagggagcttctgtgcatcgcctgagagcagcagggatgacttccaggagggaagggagggcattgtggcccggctaacagagagcctgttcctggacctgctcggagaggagaatgggggcttttgccagcaggacatgggggagtcatgccttcttccaccttcgggaagtacgagtgctcacatgccctgggatgagttcccaagtgcagggcccaaggaggcacctccctggggcaaggagcagcctctccacctggagccaagtcctcctgccagcccgacccagagtccagacaacctgacttgcacagagacgcccctcgtcatcgcaggcaaccctgcttaccgcagcttcagcaactccctgagccagtcaccgtgtcccagagagctgggtccagacccactgctggccagacacctggaggaagtagaacccgagatgccctgtgtcccccagctctctgagccaaccactgtgccccaacctgagccagaaacctgggagcagatcctccgccgaaatgtcctccagcatggggcagctgcagcccccgtctcggcccccaccagtggctatcaggagtttgtacatgcggtggagcagggtggcacccaggccagtgcggtggtgggcttgggtcccccaggagaggctggttacaaggccttctcaagcctgcttgccagcagtgctgtgtccccagagaaatgtgggtttggggctagcagtggggaagaggggtataagcctttccaagacctcattcctggctgccctggggaccctgccccagtccctgtccccttgttcacctttggactggacagggagccacctcgcagtccgcagagctcacatctcccaagcagctccccagagcacctgggtctggagccgggggaaaaggtagaggacatgccaaagcccccacttccccaggagcaggccacagacccccttgtggacagcctgggcagtggcattgtctactcagcccttacctgccacctgtgcggccacctgaaacagtgtcatggccaggaggatggtggccagacccctgtcatggccagtccttgctgtggctgctgctgtggagacaggtcctcgccccctacaacccccctgagggccccagacccctctccaggtggggttccactggaggccagtctgtgtccggcctccctggcaccctcgggcatctcagagaagagtaaatcctcatcatccttccatcctgcccctggcaatgctcagagctcaagccagacccccaaaatcgtgaactttgtctccgtgggacccacatacatgagggtctcttaggtgcatgtcctcttgttgctgagtctgcagatgaggactagggcttatccatgcctgggaaatgccacctcctggaaggcagccaggctggcagatttccaaaagacttgaagaaccatggtatgaaggtgattggccccactgacgttggcctaacactgggctgcagagactggaccccgcccagcattgggctgggctcgccacatcccatgagagtagagggcactgggtcgccgtgccccacggcaggcccctgcaggaaaactgaggcccttgggcacctcgacttgtgaacgagttgttggctgctccctccacagcttctgcagcagactgtccctgttgtaactgcccaaggcatgttttgcccaccagatcatggcccacgtggaggcccacctgcctctgtctcactgaactagaagccgagcctagaaactaacacagccatcaagggaatgacttgggcggccttgggaaatcgatgagaaattgaacttcagggagggtggtcattgcctagaggtgctcattcatttaacagagcttccttaggttgatgctggaggcagaatcccggctgtcaaggggtgttcagttaaggggagcaacagaggacatgaaaaattgctatgactaaagcagggacaatttgctgccaaacacccatgcccagctgtatggctgggggctcctcgtatgcatggaacccccagaataaatatgctcagccaccctgtgggccgggcaatccagacagcaggcataaggcaccagttaccctgcatgttggcccagacctcaggtgctagggaaggcgggaaccttgggttgagtaatgctcgtctgtgtgttttagtttcatcacctgttatctgtgtttgctgaggagagtggaacagaaggggtggagttttgtataaataaagtttctttgtctcttta

SEQ ID NO: 16 - DNA, 3486 bp, Homo sapiens
acttcccgcttgggcgcccggacggcgaatggagcaggggcgcgcaggtgccttggcatctcccaatggggtggctttgctctgggctcctgttccctgtgagctgcctggtcctgctgcaggtggcaagctctgggaacatgaaggtcttgcaggagcccacctgcgtctccgactacatgagcatctctacttgcgagtggaagatgaatggtcccaccaattgcagcaccgagctccgcctgttgtaccagctggtttttctgctctccgaagcccacacgtgtatccctgagaacaacggaggcgcggggtgcgtgtgccacctgctcatggatgacgtggtcagtgcggataactatacactggacctgtgggctgggcagcagctgctgtggaagggctccttcaagcccagcgagcatgtgaaacccagggccccaggaaacctgacagttcacaccaatgtctccgacactctgctgctgacctggagcaacccgtatccccctgacaattacctgtataatcatctcacctatgcagtcaacatttggagtgaaaacgacccggcagataatctataacgtgacctacctagaaccctccctccgcatcgcagccagcaccctgaagtctgggatttcctacagggcacgggtgagggcctgggctcagtgctataacaccacctggagtgagtggagccccagcaccaagtggcacaactcctacagggagcccttcgagcagcacctcctgctgggcgtcagcgtttcctgcattgtcatcctggccgtctgcctgttgtgctatgtcagcatcaccaagattaagaaagaatggtgggatcagattcccaacccagcccgcagccgcctcgtggctataataatccaggatgctcaggggtcacagtgggagaagcggtcccgaggccaggaaccagccaagtgcccacactggaagaattgtcttaccaagctcttgccctgttttctggagcacaacatgaaaagggatgaagatcctcacaaggctgccaaagagatgcctttccagggctctggaaaatcagcatggtgcccagtggagatcagcaagacagtcctctggccagagagcatcagcgtggtgcgatgtgtggagttgtttgaggccccggtggagtgtgaggaggaggaggaggtagaggaagaaaaagggagcttctgtgcatcgcctgagagcagcagggatgacttccaggagggaagggagggcattgtggcccggctaacagagagcctgttcctggacctgctcggagaggagaatgggggcttttgccagcaggacatgggggagtcatgccttcttccaccttcgggaagtacgagtgctcacatgccctgggatgagttcccaagtgcagggcccaaggaggcacctccctggggcaaggagcagcctctccacctggagccaagtcctcctgccagcccgacccagagtccagacaacctgacttgcacagagacgcccctcgtcatcgcaggcaaccctgcttaccgcagcttcagcaactccctgagccagtcaccgtgtcccagagagctgggtccagacccactgctggccagacacctggaggaagtagaacccgagatgccctgtgtcccccagctctctgagccaaccactgtgccccaacctgagccagaaacctgggagcagatcctccgccgaaatgtcctccagcatggggcagctgcagcccccgtctcggcccccaccagtggctatcaggagtttgtacatgcggtggagcagggtggcacccaggccagtgcggtggtgggcttgggtcccccaggagaggctggttacaaggccttctcaagcctgcttgccagcagtgctgtgtccccagagaaatgtgggtttggggctagcagtggggaagaggggtataagcctttccaagacctcattcctggctgccctggggaccctgccccagtccctgtccccttgttcacctttggactggacagggagccacctcgcagtccgcagagctcacatctcccaagcagctccccagagcacctgggtctggagccgggggaaaaggtagaggacatgccaaagcccccacttccccaggagcaggccacagacccccttgtggacagcctgggcagtggcattgtctactcagcccttacctgccacctgtgcggccacctgaaacagtgtcatggccaggaggatggtggccagacccctgtcatggccagtccttgctgtggctgctgctgtggagacaggtcctcgccccctacaacccccctgagggccccagacccctctccaggtggggttccactggaggccagtctgtgtccggcctccctggcaccctcgggcatctcagagaagagtaaatcctcatcatccttccatcctgcccctggcaatgctcagagctcaagccagacccccaaaatcgtgaactttgtctccgtgggacccacatacatgagggtctcttaggtgcatgtcctcttgttgctgagtctgcagatgaggactagggcttatccatgcctgggaaatgccacctcctggaaggcagccaggctggcagatttccaaaagacttgaagaaccatggtatgaaggtgattggccccactgacgttggcctaacactgggctgcagagactggaccccgcccagcattgggctgggctcgccacatcccatgagagtagagggcactgggtcgccgtgccccacggcaggcccctgcaggaaaactgaggcccttgggcacctcgacttgtgaacgagttgttggctgctccctccacagcttctgcagcagactgtccctgttgtaactgcccaaggcatgttttgcccaccagatcatggcccacgtggaggcccacctgcctctgtctcactgaactagaagccgagcctagaaactaacacagccatcaagggaatgacttgggcggccttgggaaatcgatgagaaattgaacttcagggagggtggtcattgcctagaggtgctcattcatttaacagagcttccttaggttgatgctggaggcagaatcccggctgtcaaggggtgttcagttaaggggagcaacagaggacatgaaaaattgctatgactaaagcagggacaatttgctgccaaacacccatgcccagctgtatggctgggggctcctcgtatgcatggaacccccagaataaatatgctcagccaccctgtgggccgggcaatccagacagcaggcataaggcaccagttaccctgcatgttggcccagacctcaggtgctagggaaggcgggaaccttgggttgagtaatgctcgtctgtgtgttttagtttcatcacctgttatctgtgtttgctgaggagagtggaacagaaggggtggagttttgtataaataaagtttctttgtctcttta

SEQ ID NO: 17 - DNA, 1127 bp, Homo sapiens
attctgccctcgagcccaccgggaacgaaagagaagctctatctcccctccaggagcccagctatgaactccttctccacaagcgccttcggtccagttgccttctccctggggctgctcctggtgttgcctgctgccttccctgccccagtacccccaggagaagattccaaagatgtagccgccccacacagacagccactcacctcttcagaacgaattgacaaacaaattcggtacatcctcgacggcatctcagccctgagaaaggagacatgtaacaagagtaacatgtgtgaaagcagcaaagaggcactggcagaaaacaacctgaaccttccaaagatggctgaaaaagatggatgcttccaatctggattcaatgaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatgtagcatgggcacctcagattgttgttgttaatgggcattccttcttctggtcagaaacctgtccactgggcacagaacttatgttgttctctatggagaactaaaagtatgagcgttaggacactattttaattatttttaatttattaatatttaaatatgtgaagctgagttaatttatgtaagtcatatttatatttttaagaagtaccacttgaaacattttatgtattagttttgaaataataatggaaagtggctatgcagtttgaatatcctttgtttcagagccagatcatttcttggaaagtgtaggcttacctcaaataaatggctaacttatacatatttttaaagaaatatttatattgtatttatataatgtataaatggtttttataccaataaatggcattttaaaaaattca

SEQ ID NO: 18 - DNA, 936 bp, Homo sapiens
attctgccctcgagcccaccgggaacgaaagagaagctctatctcccctccaggagcccagctatgaactccttctccacaaacatgtaacaagagtaacatgtgtgaaagcagcaaagaggcactggcagaaaacaacctgaaccttccaaagatggctgaaaaagatggatgcttccaatctggattcaatgaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatgtagcatgggcacctcagattgttgttgttaatgggcattccttcttctggtcagaaacctgtccactgggcacagaacttatgttgttctctatggagaactaaaagtatgagcgttaggacactattttaattatttttaatttattaatatttaaatatgtgaagctgagttaatttatgtaagtcatatttatatttttaagaagtaccacttgaaacattttatgtattagttttgaaataataatggaaagtggctatgcagtttgaatatcctttgtttcagagccagatcatttcttggaaagtgtaggcttacctcaaataaatggctaacttatacatatttttaaagaaatatttatattgtatttatataatgtataaatggtttttataccaataaatggcattttaaaaaattca

SEQ ID NO: 19 - DNA, 1058 bp, Homo sapiens
attctgccctcgagcccaccgggaacgaaagagaagctctatctcccctccaggagcccagctatgaactccttctccacaatacccccaggagaagattccaaagatgtagccgccccacacagacagccactcacctcttcagaacgaattgacaaacaaattcggtacatcctcgacggcatctcagccctgagaaaggagacatgtaacaagagtaacatgtgtgaaagcagcaaagaggcactggcagaaaacaacctgaaccttccaaagatggctgaaaaagatggatgcttccaatctggattcaatgaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatgtagcatgggcacctcagattgttgttgttaatgggcattccttcttctggtcagaaacctgtccactgggcacagaacttatgttgttctctatggagaactaaaagtatgagcgttaggacactattttaattatttttaatttattaatatttaaatatgtgaagctgagttaatttatgtaagtcatatttatatttttaagaagtaccacttgaaacattttatgtattagttttgaaataataatggaaagtggctatgcagtttgaatatcctttgtttcagagccagatcatttcttggaaagtgtaggcttacctcaaataaatggctaacttatacatatttttaaagaaatatttatattgtatttatataatgtataaatggtttttataccaataaatggcattttaaaaaattca